(19) United States
(12) Patent Application Publication
(10) Pub. No.: US 2023/0041822 A1
(43) Pub. Date: Feb. 9, 2023

(54) RECOMBINANT PEPTIDE-MHC COMPLEX BINDING PROTEINS AND THEIR GENERATION AND USE

(71) Applicant: MOLECULAR PARTNERS AG, Zurich-Schlieren (CH)

(72) Inventors: Victor LEVITSKY, Birmensdorf (CH); Natalia VENETZ, Zurich (CH); Marcel WALSER, Winterthur (CH)

(21) Appl. No.: 17/757,183

(22) PCT Filed: Dec. 11, 2020
(86) PCT No.: PCT/EP2020/085864

§ 371 Date: Jun. 10, 2022

(30) Foreign Application Priority Data
Dec. 11, 2019 (EP) 19215436.7

(51) Int. Cl. C12N 15/10; C07K 14/47

(52) U.S. Cl. CPC C12N 15/1041; C07K 14/4702; A61K 38/00

(57) ABSTRACT

The present invention relates to a method of producing recombinant binding proteins with binding specificity for a peptide-MHC (pMHC) complex. The invention also relates to recombinant binding proteins comprising one, two or more designed repeat domain(s), preferably designed ankyrin repeat domain(s), with binding specificity for a pMHC complex, and to such binding proteins which further comprise a binding agent having binding specificity for a protein expressed on the surface of an immune cell, preferably a T-cell. In addition, the invention relates to nucleic acids encoding such binding proteins or repeat domains, pharmaceutical compositions comprising such binding proteins or nucleic acids, and the use of such binding proteins, nucleic acids or pharmaceutical compositions in methods for treating or diagnosing diseases, including cancer, infectious diseases and autoimmune diseases.

### CROSS-REFERENCE TO RELATED APPLICATIONS

The present application claims the benefit of priority to EP19215436.7, filed on Dec. 11, 2019. The disclosure of this patent application is incorporated herein for all purposes by reference in its entirety.

### FIELD OF THE DISCLOSURE

The present invention relates to a method of producing recombinant binding proteins with binding specificity for a peptide-MHC (pMHC) complex. The invention also relates to recombinant binding proteins comprising one, two or more designed repeat domain(s), preferably designed ankyrin repeat domain(s), with binding specificity for a pMHC complex, and to such binding proteins which further comprise a binding agent having binding specificity for a protein expressed on the surface of an immune cell, preferably a T-cell. In addition, the invention relates to nucleic acids encoding such binding proteins or repeat domains, pharmaceutical compositions comprising such binding proteins or nucleic acids, and the use of such binding proteins, nucleic acids or pharmaceutical compositions in methods for treating or diagnosing diseases, including cancer, infectious diseases and autoimmune diseases.

### BACKGROUND

Major histocompatibility complex (MHC) class I molecules play a key role in the surveillance of aberrant or foreign proteins within cells. Peptides derived from endogenous proteins are loaded onto the peptide-binding groove of MHC class I molecules and are then displayed on the cell surface. Such MHC class I complexes are expressed in all nucleated cells, including malignant cells. Peptide-MHC (pMHC) complexes are recognized by T cell receptors (TCRs) on CD8+ cytotoxic T lymphocytes (CTLs). The presentation of peptide-MHC complexes provides a snapshot of the intracellular environment to circulating CTLs (Reeves and James, Immunology 150: 16-24 (2016)). CTLs are activated in response to detecting aberrant or foreign antigens, e.g. oncoproteins or bacterial or viral proteins, resulting in the destruction of the presenting cell. CTLs are also activated in certain autoimmune diseases upon misrecognizing a “self” antigen (Bodis et al., Rheumatol. Ther. 5: 5-20 (2018)).

MHC class I complexes presenting tumor-specific or infectious agent-specific peptides represent a unique and promising class of cell surface targets for immunotherapy of cancer and infectious diseases. Different approaches have been developed in attempts to exploit this target class, including vaccines, adoptive cell therapy and TCR-like antibodies. Among the infectious agent-specific peptides, the sequence 18-27 of the hepatitis B virus (HBV) nucleocapsid antigen is widely recognized by CTL of HLA-A2-positive patients with acute self-limited HBV infection, and represents the main component of a peptide-based therapeutic vaccine aimed at stimulating the antiviral CTL response in patients with chronic hepatitis B (Hepatology. 1997 October; 26(4):1027-34.c).

One obstacle to the therapeutic exploitation of virus- or tumor-specific peptide-MHC complexes has been the inherently low affinity of TCRs for peptide-MHC complexes after thymic selection. This low affinity presents a limitation, especially considering that virus- or tumor-specific peptide-MHC complexes are typically present on the surface of virus-infected or tumor cells in low density. To overcome this obstacle, affinity-enhanced TCRs have been developed and engineered T-cells expressing such affinity-enhanced TCRs have been tested in clinical studies. It has been reported that affinity-enhanced TCRs can lack specificity, and, in some instances, engineered T-cells expressing affinity-enhanced TCRs have caused serious, sometimes even deadly, medical complications due to the unexpected recognition by the TCRs of an epitope derived from an unrelated protein (see, e.g., Linette et al., Blood 122(6): 863-871 (2013)). Affinity-enhanced TCRs are also being developed as soluble TCRs, but soluble TCR expression is challenging.

A number of TCR-like antibodies with binding specificity for MHC class I complexes presenting viral- or tumor-specific peptides have been reported. Some of them were isolated using hybridoma technology. However, isolation of pMHC-specific TCR-like antibodies by hybridoma technology has been hampered by a number of factors, including the need to screen hundreds or even thousands of clones, low immunogenicity, few unique clones due to immunodominance, and poor control of fine-specificity (see, e.g., Porgador et al., Immunity 6: 715-726 (1997); Bernardeau et al., Eur. J. Immunol. 35(10): 2864-2875 (2005); Skora et al., Proc. Natl. Acad. Sci. USA 112(32): 9967-9972 (2015)). More TCR-like antibodies have been isolated using phage display. However, the affinity of TCR-like antibodies isolated from phage display libraries is generally relatively low and often not sufficient for therapeutic purposes (see, e.g., Chames et al., Proc. Natl. Acad. Sci. USA 97: 7969-7974 (2000)). In order to generate TCR-like antibodies with sufficiently high affinity, systems for affinity maturation have been developed. For example, one such system for affinity maturation of a TCR-like antibody combines mutagenesis, libraries and yeast display, and structure determinations and molecular modeling (Zhao et al., Leukemia 29(11): 2238-2247 (2015)). Only with such complex and labor-intensive affinity maturation approach were Zhao et al. able to improve the binding affinity of a TCR-like antibody by about 100-fold to obtain a pMHC-specific binding protein with sufficient affinity. Taken together, it has been challenging thus far to develop molecules that specifically bind disease-related peptide-MHC complexes with sufficient affinity, and current approaches generally involve difficult expression systems and/or time- and labor-consuming procedures such as screening of a large number of hybridoma clones or affinity maturation.

Thus, there still remains a need for new methods of producing pMHC-specific binding proteins, for new pMHC-specific binding proteins, and for therapeutic and diagnostic approaches for the treatment and characterization of diseases, including cancer, autoimmune diseases and infectious diseases, benefitting from pMHC-specific binding.

### SUMMARY

The present invention provides a method of producing recombinant binding proteins with binding specificity for a peptide-MHC (pMHC) complex. The invention also provides recombinant binding proteins comprising one, two or more designed repeat domain(s), preferably designed ankyrin repeat domain(s), with binding specificity for a pMHC complex, and such binding proteins which further comprise a binding agent having binding specificity for a protein expressed on the surface of an immune cell, preferably a T-cell. In addition, the invention provides nucleic acids encoding such binding proteins or repeat domains, pharmaceutical compositions comprising such binding proteins or nucleic acids, and the use of such binding proteins, nucleic acids or pharmaceutical compositions in methods for treating or diagnosing diseases, such, e.g., as cancer, autoimmune diseases and infectious diseases, in a mammal, including a human.

The inventive method of producing recombinant binding proteins with binding specificity for a pMHC complex is surprisingly efficient and effective in generating binding proteins that bind with high affinity and/or specificity to a chosen target pMHC complex. Thus far it has been challenging to develop molecules that specifically bind disease-related pMHC complexes with sufficient affinity, and current approaches generally involve difficult expression systems and/or time- and labor-consuming procedures such as screening of a large number of hybridoma clones or affinity maturation. The inventive method disclosed herein neither involves a difficult expression system nor does it require time- and labor-consuming procedures such as those mentioned above. Furthermore, the binding interaction between binding proteins of the invention and the target peptide-MHC complex unexpectedly involves a relatively large number of amino acid residues in the target peptide. A large number of interaction residues in the target peptide is believed to reflect a highly selective or specific binding interaction with the target peptide-MHC complex within the large universe of different pMHC complexes. It appears that the pMHC-specific repeat domain(s) of the binding proteins of the invention may provide a binding surface that sterically fits very well to the composite peptide-MHC surface in a specific binding interaction. The methods and binding proteins of the invention further provide the advantage that two or more of the same and/or different pMHC-specific repeat domains can be readily combined in one binding protein (generating, e.g., bivalent, biparatopic or bispecific binding proteins), thereby allowing to adapt and optimize binding avidity, binding affinity, binding specificity and/or potency of the binding proteins. Moreover, binding proteins of the invention may even further comprise a binding agent with binding specificity for a protein expressed on the surface of an immune cell, such as, e.g., a protein that is part of the T cell receptor complex expressed in cytotoxic T cells. Binding proteins of the invention in such an immune cell engager format (e.g. a T cell engager format) can be advantageously used in methods to activate immune cells (e.g. T cells) and/or to engage the immune system in a localized and targeted fashion. Furthermore, the methods and binding proteins of the invention allow to specifically target intracellular proteins, among others, thereby facilitating many new diagnostic and therapeutic opportunities, e.g. for cancers, infectious diseases and autoimmune diseases. In one aspect, the invention provides a method of producing a peptide-MHC (pMHC)-specific binding protein, wherein said binding protein comprises a designed repeat domain with binding specificity for a target peptide-MHC complex, the method comprising the steps of:

(a) providing a collection of designed repeat domains;

(b) providing a recombinant target peptide-MHC complex; and

(c) screening said collection of designed repeat domains for specific binding to said target peptide-MHC complex to obtain at least one designed repeat domain with binding specificity for said target peptide-MHC complex. In a preferred embodiment, said designed repeat domain is a designed ankyrin repeat domain.

In another aspect, the invention provides a recombinant binding protein comprising a designed repeat domain obtainable by the above method.

In another aspect, the invention provides a recombinant binding protein comprising a first designed repeat domain, wherein said first repeat domain has binding specificity for a first target peptide-MHC complex. In a preferred embodiment said first target peptide is derived from a protein associated with a disease or disorder. As examples, in one particular preferred embodiment, said first target peptide is selected from the group consisting of (i) a peptide derived from a protein expressed in a tumor cell, (ii) a peptide derived from a protein of an infectious agent, preferably a viral infectious agent, and (iii) a peptide derived from a protein associated with an autoimmune disorder.

In one particular aspect, said first target peptide is derived from a protein of a viral infectious agent, preferably a virus-specific protein such as, e.g., HBV core antigen (HBcAg). In one preferred aspect, a target peptide which is derived from HBcAg comprises or consists of the amino acid sequence of SEQ ID NO: 51.

In one preferred aspect, the invention provides such a recombinant binding protein comprising a first designed repeat domain with binding specificity for a first target peptide-MHC complex, wherein said first repeat domain is a designed ankyrin repeat domain. In one particular aspect, the invention provides such a recombinant binding protein comprising a first ankyrin repeat domain with binding specificity for a first target peptide-MHC complex, wherein said first target peptide is derived from HBcAg, and wherein said first ankyrin repeat domain comprises an ankyrin repeat module comprising an amino acid sequence selected from the group consisting of (1) SEQ ID NOs: 27 to 50 and (2) sequences in which up to 9 amino acids in any of SEQ ID NOs: 27 to 50 are substituted by another amino acid. In one particular embodiment, the invention provides such a recombinant binding protein comprising a first ankyrin repeat domain with binding specificity for a first target peptide-MHC complex, wherein said first target peptide is derived from HBcAg, and wherein said first ankyrin repeat domain comprises an amino acid sequence with at least 80% amino acid sequence identity with any one of SEQ ID NOs: 16 to 26, wherein G at position 1 and/or S at position 2 of SEQ ID NOs: 16 to 26 are optionally missing, and wherein A at the second last position of SEQ ID NOs: 16 to 26 is optionally substituted by L and/or A at the last position of SEQ ID NOs: 16 to 26 is optionally substituted by N.

In one particular aspect, the invention provides such a recombinant binding protein further comprising a second designed repeat domain with binding specificity for a second target peptide-MHC complex. In a preferred embodiment said second target peptide is derived from a protein associated with a disease or disorder. As examples, in one particular preferred embodiment, said second target peptide is selected from the group consisting of (i) a peptide derived from a protein expressed in a tumor cell, (ii) a peptide derived from a protein of an infectious agent, preferably a viral infectious agent, and (iii) a peptide derived from a protein associated with an autoimmune disorder.

In one particular aspect, said second target peptide is derived from the same protein as said first target peptide. In one embodiment, said second target peptide has the same amino acid sequence as said first target peptide. In one embodiment, said second repeat domain has the same amino acid sequence as said first repeat domain. In one embodiment, said second repeat domain has a different amino acid sequence as compared to said first repeat domain. In one embodiment, said second target peptide has a different amino acid sequence as compared to said first target peptide.

In one particular aspect, said second target peptide is derived from a protein that is different from the protein, from which said first target peptide is derived.

In one preferred aspect, the invention provides such a recombinant binding protein further comprising a second designed repeat domain with binding specificity for a second target peptide-MHC complex, wherein said second repeat domain is a designed ankyrin repeat domain. In one particular aspect, the invention provides such a recombinant binding protein further comprising a second ankyrin repeat domain with binding specificity for a second target peptide-MHC complex, wherein said second target peptide is derived from HBcAg, and wherein said second ankyrin repeat domain comprises an ankyrin repeat module comprising an amino acid sequence selected from the group consisting of (1) SEQ ID NOs: 27 to 50 and (2) sequences in which up to 9 amino acids in any of SEQ ID NOs: 27 to 50 are substituted by another amino acid. In one particular embodiment, the invention provides such a recombinant binding protein further comprising a second ankyrin repeat domain with binding specificity for a second target peptide-MHC complex, wherein said second target peptide is derived from HBcAg, and wherein said second ankyrin repeat domain comprises an amino acid sequence with at least 80% amino acid sequence identity with any one of SEQ ID NOs: 16 to 26, wherein G at position 1 and/or S at position 2 of SEQ ID NOs: 16 to 26 are optionally missing, and wherein A at the second last position of SEQ ID NOs: 16 to 26 is optionally substituted by L and/or A at the last position of SEQ ID NOs: 16 to 26 is optionally substituted by N.

In one preferred aspect, binding of the repeat domain to its target peptide-MHC complex comprises interaction of said repeat domain with at least one, at least two, at least three, at least four, at least five, at least six, or at least seven amino acid residues of said target peptide. In one embodiment, binding of the repeat domain to its target peptide-MHC complex alternatively or further comprises interaction of said repeat domain with at least one amino acid residue of said MHC.

In one particular aspect, the invention provides such pMHC-specific recombinant binding proteins, wherein the binding proteins further comprise a binding agent with binding specificity for a protein expressed on the surface of an immune cell, preferably a T-cell, more preferably a CD8+ cytotoxic T-cell. In one embodiment, said protein expressed on the surface of a T-cell is a protein that is part of the T-cell receptor complex. As an example, in one particular embodiment, the invention provides such pMHC-specific recombinant binding proteins, wherein the binding proteins further comprise a binding agent with binding specificity for CD3.

In one preferred aspect, the invention provides such a recombinant binding protein further comprising a binding agent with binding specificity for a protein expressed on the surface of an immune cell, wherein said binding agent is a designed repeat domain, preferably a designed ankyrin repeat domain.

In another aspect, the invention provides nucleic acids encoding the designed repeat domains of the invention or encoding the pMHC-specific recombinant binding proteins of the invention and pharmaceutical compositions comprising the pMHC-specific recombinant binding protein or nucleic acid of the invention and a pharmaceutically acceptable carrier and/or diluent.

In another aspect, the invention provides a method of infection-localized activation of T-cells in a mammal, preferably a human, the method comprising the step of administering to said mammal the pMHC-specific recombinant binding protein or nucleic acid of the invention, wherein said binding protein further comprises a binding agent with binding specificity for a protein expressed on the surface of an immune cell, and wherein said first target peptide and/or, if said binding protein comprises said second repeat domain, said second target peptide is derived from a protein of an infectious agent.

In another aspect, the invention provides the pMHC-specific recombinant binding protein or nucleic acid according to the invention for use in a method of infection-localized activation of T-cells in a mammal, preferably a human, wherein said binding protein further comprises a binding agent with binding specificity for a protein expressed on the surface of an immune cell, and wherein said first target peptide and/or, if said binding protein comprises said second repeat domain, said second target peptide is derived from a protein of an infectious agent.

In another aspect, the invention provides a method for treating a medical condition, the method comprising the step of administering to a patient in need thereof a therapeutically effective amount of the pMHC-specific recombinant binding protein, nucleic acid, or pharmaceutical composition of the invention.

In another aspect, the invention provides the pMHC-specific recombinant binding protein, nucleic acid, or pharmaceutical composition of the invention for use in a method of treating a medical condition.

In another aspect, the invention provides a method of diagnosing a medical condition in a mammal, preferably a human, the method comprising the steps of:

(i) contacting a cell or tissue sample obtained from said mammal with the pMHC-specific recombinant binding protein of the invention; and

(ii) detecting specific binding of said binding protein to said cell or tissue sample.

In one particular aspect, said medical condition is a cancer, an infectious disease, preferably a viral infectious disease, or an autoimmune disease. In one embodiment, said medical condition is a cancer. In one embodiment, said medical condition is an infectious disease, preferably a viral infectious disease. In one embodiment, said medical condition is an autoimmune disease.

In another aspect, the invention provides a method of targeting infected cells in a patient having a viral infectious disease for destruction of the infected cells, the method comprising the step of administering to the patient a therapeutically effective amount the pMHC-specific recombinant binding protein, nucleic acid, or pharmaceutical composition of the invention, wherein said first target peptide and/or, if said binding protein comprises said second repeat domain, said second target peptide is derived from a protein expressed in the infected cells, preferably a virus-specific protein. In one embodiment, said binding protein further comprises a toxic agent capable of killing an infected cell.

In another aspect, the invention provides the pMHC-specific recombinant binding protein, nucleic acid, or pharmaceutical composition of the invention for use in a method of targeting infected cells in a patient having a viral infectious disease for destruction of the infected cells, wherein said first target peptide and/or, if said binding protein comprises said second repeat domain, said second target peptide is derived from a protein expressed in the infected cells, preferably a virus-specific protein. In one embodiment, said binding protein further comprises a toxic agent capable of killing an infected cell.

### BRIEF DESCRIPTION OF THE FIGURES

FIGS. 1A to 1J: High affinity binding of binding proteins of the invention to target peptide-MHC complex. Multi-trace SPR measurement for DARPin® proteins #16 (A), #17 (B), #18 (C), #20 (D), #21 (E), #22 (F), #23 (G), #24 (H), #25 (I) and #26 (J). The DARPin® proteins tested bound with high affinity to HBVc18pMHC.

FIGS. 2A to 2J: T cell activation assay using HBVc18-pulsed (10 nM) T2 cells as target cells (black bars), compared to T cell activation using non-pulsed T2 cells (dark grey bars) or HLA-A*02+ T cells only as controls (light grey bars). Three DARPin concentrations were used (10,000 pM, 100 pM and 1 pM). The tested TCE DARPin® proteins were: (A) TCE DARPin® protein #16, (B) TCE DARPin® protein #17, (C) TCE DARPin® protein #18, (D) TCE DARPin® protein #20, (E) TCE DARPin® protein #21, (F) TCE DARPin® protein #22, (G) TCE DARPin® protein #23, (H) TCE DARPin® protein #24, (I) TCE DARPin® protein #25, and (J) TCE DARPin® protein #26. Intracellular interferon-γ (IFN-γ) in the T cells was detected by FACS.

FIGS. 3A to 3J: Target peptide binding analyzed by alanine scanning mutagenesis. Functional binding of binding proteins to the HBVc18 peptide (c18) and a series of alanine-mutated variants thereof were tested in a T cell activation assay using pulsed T2 cells as target cells and BK112 T cells as effector cells. The amino acid residues and positions in the HBVc18 peptide that were replaced with alanine are indicated. As a control, non-pulsed T2 cells were used. The tested TCE DARPin® proteins were: (A) TCE DARPin® protein #16, (B) TCE DARPin® protein #17, (C) TCE DARPin® protein #18, (D) TCE DARPin® protein #20, (E) TCE DARPin® protein #21, (F) TCE DARPin® protein #22, (G) TCE DARPin® protein #23, (H) TCE DARPin® protein #24, (I) TCE DARPin® protein #25, and (J) TCE DARPin® protein #26. Intracellular IFN-γ was detected as a measure of T cell activation.

FIGS. 4 and 5: T cell activation by pMHC-specific binding proteins in TCE format depending on length of linker connecting the pMHC-specific binding domain and the CD3-specific binding agent. HLA-A2+/NY-ESO-1⁺ tumor cells (IM9) (solid lines) or HLA-A2+/NY-ESO-1⁻ tumor cells (MCF-7) (dashed lines) were incubated with PBMCs for 48 hours in the presence or absence of TCE DARPin® protein #78 with different linker lengths (FIG. 4) or of TCE DARPin® protein #79 with different linker lengths (FIG. 5). After 48 hours, CD25 expression was measured on CD8⁺ T cells. Results obtained in the presence of TCE DARPin® protein are shown. No T cell activation was observed in the absence of TCE DARPin® protein. Ag: antigen/NY-ESO-1. Linker lengths: standard, XXS, XS, S and L (see Example 5).

FIGS. 6 and 7: T cell activation by pMHC-specific binding proteins in TCE format depending on length of linker connecting the pMHC-specific binding domain and the CD3-specific binding agent. HLA-A2+/NY-ESO-1⁺ tumor cells (U266B1) (solid lines) or HLA-A2+/NY-ESO-1⁻ tumor cells (Colo205) (dashed lines) were incubated with PBMCs for 48 hours in the presence or absence of TCE DARPin® protein #78 with different linker lengths (FIG. 6) or of TCE DARPin® protein #79 with different linker lengths (FIG. 7). After 48 hours, CD25 expression was measured on CD8⁺ T cells. Results obtained in the presence of TCE DARPin® protein are shown. No T cell activation was observed in the absence of TCE DARPin® protein. Ag: antigen/NY-ESO-1. Linker lengths: standard, XXS, XS, S and L (see Example 5).

### DETAILED DESCRIPTION OF THE INVENTION

As disclosed and exemplified herein, the disclosure provides designed repeat proteins, preferably designed ankyrin repeat proteins, that specifically target a peptide-MHC complex. Designed repeat protein libraries, including designed ankyrin repeat protein libraries (WO2002/020565; Binz et al., Nat. Biotechnol. 22, 575-582, 2004; Stumpp et al., Drug Discov. Today 13, 695-701, 2008), can be used for the selection of target-specific designed repeat domains that bind to their target with high affinity. Such target-specific designed repeat domains in turn can be used as valuable components of recombinant binding proteins for the treatment of diseases. Whether designed repeat protein libraries can be used to identify proteins that specifically and with high affinity bind to a composite epitope, such as the ones presented by peptide-MHC complexes, had not been shown. Generating molecules that specifically bind disease-related peptide-MHC complexes with sufficient affinity has been notoriously difficult.

Designed ankyrin repeat proteins are a class of binding molecules which have the potential to overcome limitations of monoclonal antibodies, hence allowing novel therapeutic approaches. Such ankyrin repeat proteins may comprise a single designed ankyrin repeat domain, or may comprise a combination of two, three, four, five or more designed ankyrin repeat domains with the same or different target specificities (Stumpp et al., Drug Discov. Today 13, 695-701, 2008; U.S. Pat. No. 9,458,211). Ankyrin repeat proteins comprising only a single designed ankyrin repeat domain are small proteins (14 kDa) which can be selected to bind a given target protein with high affinity and specificity. These characteristics, and the possibility of combining two, three, four, five or more designed ankyrin repeat domains in one protein, make designed ankyrin repeat proteins ideal agonistic, antagonistic and/or inhibitory drug candidates. Furthermore, such ankyrin repeat proteins can be engineered to carry various effector functions, e.g. cytotoxic agents or half-life extending agents, enabling completely new drug formats. Taken together, designed ankyrin repeat proteins are an example of the next generation of protein therapeutics with the potential to surpass existing antibody drugs.

DARPin® is a trademark owned by Molecular Partners AG, Switzerland.

In one aspect, the invention provides a method of producing a peptide-MHC (pMHC)-specific binding protein, wherein said binding protein comprises a designed repeat domain with binding specificity for a target peptide-MHC complex, the method comprising the steps of:
(a) providing a collection of designed repeat domains;
(b) providing a recombinant target peptide-MHC complex; and
(c) screening said collection of designed repeat domains for specific binding to said target peptide-MHC complex to obtain at least one designed repeat domain with binding specificity for said target peptide-MHC complex.

In one embodiment, said designed repeat domain is a designed ankyrin repeat domain and said collection of designed repeat domains is a collection of designed ankyrin repeat domains.

In one embodiment, said collection comprises designed repeat domains that comprise fixed positions and randomized positions, and wherein designed repeat domains of said collection differ from each other in at least one of the randomized positions.

In one embodiment, said collection of designed repeat proteins is provided by ribosome display.

In one embodiment, the method further comprises the steps of (i) providing a second recombinant peptide-MHC complex, wherein said peptide of said second peptide-MHC complex comprises an amino acid sequence that differs from said target peptide by at least one amino acid residue; and (ii) removing from said collection by negative selection designed repeat domains with binding specificity for said second recombinant peptide-MHC complex.

In one embodiment, said repeat domain with binding specificity for a target peptide-MHC complex binds to said target peptide-MHC complex in PBS with a dissociation constant (K_{D}) below 10⁻⁷M, or below 5×10⁻⁸M, or below 3×10⁻⁸M, or below 2×10⁻⁸M, or below 10⁻⁸M, or below 5×10⁻⁹M, or below 3×10⁻⁹M, or below 2×10⁻⁹M, or below 10⁻⁹M, or below 5×10⁻¹⁰M, or below 3×10⁻¹⁰M, or below 2×10⁻¹⁰M, or below 10⁻¹⁰M. In one embodiment, said repeat domain with binding specificity for a target peptide-MHC complex binds to said target peptide-MHC complex in PBS with a dissociation constant (K_{D}) below 10⁻⁷M.

In one embodiment, binding of said repeat domain with binding specificity fora target peptide-MHC complex to said target peptide-MHC complex comprises interaction of said repeat domain with at least one, at least two, at least three, at least four, at least five, at least six, or at least seven amino acid residues of said target peptide. Thus, in one embodiment, binding of said repeat domain with binding specificity for a target peptide-MHC complex to said target peptide-MHC complex comprises interaction of said repeat domain with at least one amino acid residue of said target peptide. In one embodiment, binding of said repeat domain with binding specificity for a target peptide-MHC complex to said target peptide-MHC complex comprises interaction of said repeat domain with at least two amino acid residues of said target peptide. In one embodiment, binding of said repeat domain with binding specificity for a target peptide-MHC complex to said target peptide-MHC complex comprises interaction of said repeat domain with at least three amino acid residues of said target peptide. In one embodiment, binding of said repeat domain with binding specificity for a target peptide-MHC complex to said target peptide-MHC complex comprises interaction of said repeat domain with at least four amino acid residues of said target peptide. In one embodiment, binding of said repeat domain with binding specificity for a target peptide-MHC complex to said target peptide-MHC complex comprises interaction of said repeat domain with at least five amino acid residues of said target peptide. In one embodiment, binding of said repeat domain with binding specificity for a target peptide-MHC complex to said target peptide-MHC complex comprises interaction of said repeat domain with at least six amino acid residues of said target peptide. In one embodiment, binding of said repeat domain with binding specificity for a target peptide-MHC complex to said target peptide-MHC complex comprises interaction of said repeat domain with at least seven amino acid residues of said target peptide. In a further or alternative embodiment, binding of said repeat domain with binding specificity for a target peptide-MHC complex to said target peptide-MHC complex comprises interaction of said repeat domain with at least one amino acid residue of said MHC.

Methods to determine the amino acid residues involved in the binding interaction between proteins or between a protein and a peptide, such as, e.g., alanine scanning mutagenesis, are well known to the person skilled in the art. As an example, the amino acid residues of a target peptide that are involved in the binding interaction between a binding protein or a designed repeat domain of the invention with binding specificity for a target peptide-MHC complex and the target peptide-MHC complex may be determined by alanine scanning mutagenesis.

In the context of the present invention, a typical and preferred determination of the amino acid residues of a target peptide that are involved in the binding interaction between a binding protein or a designed repeat domain with binding specificity for a target peptide-MHC complex and the target peptide-MHC complex is performed by alanine scanning mutagenesis as described in Example 4. Thus, in one embodiment said amino acid residues of a target peptide that are involved in the binding interaction between a binding protein or a designed repeat domain of the invention with binding specificity for a target peptide-MHC complex and the target peptide-MHC complex are determined by alanine scanning mutagenesis. In one embodiment said amino acid residues of a target peptide that are involved in the binding interaction between a binding protein or a designed repeat domain of the invention with binding specificity for a target peptide-MHC complex and the target peptide-MHC complex are determined by alanine scanning mutagenesis as described in Example 4.

In the context of the present invention, the phrase “binding of said repeat domain with binding specificity for a target peptide-MHC complex to said target peptide-MHC complex comprises interaction of said repeat domain with at least one, at least two, at least three, at least four, at least five, at least six, or at least seven amino acid residues of said target peptide”, or similar phrases, refers to any amino acid residues of the target peptide, the mutation of which to alanine results in a reduction of T cell activation in an alanine scanning mutagenesis assay as described in Example 4 (or a similar assay) by at least 50% compared to the wild-type peptide.

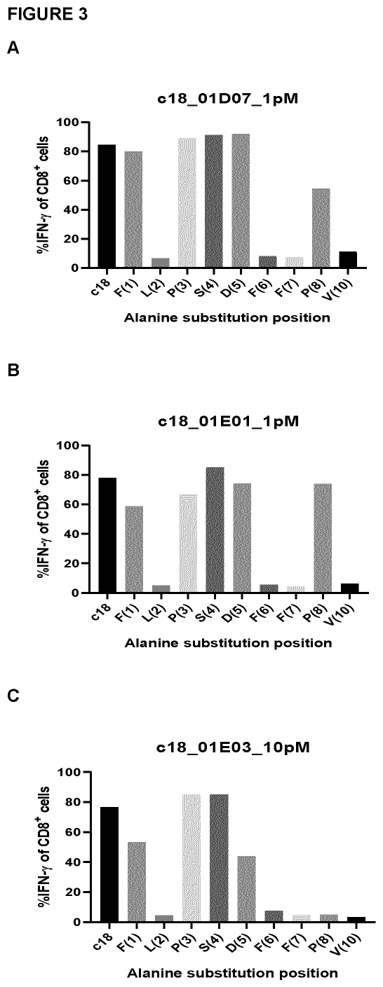

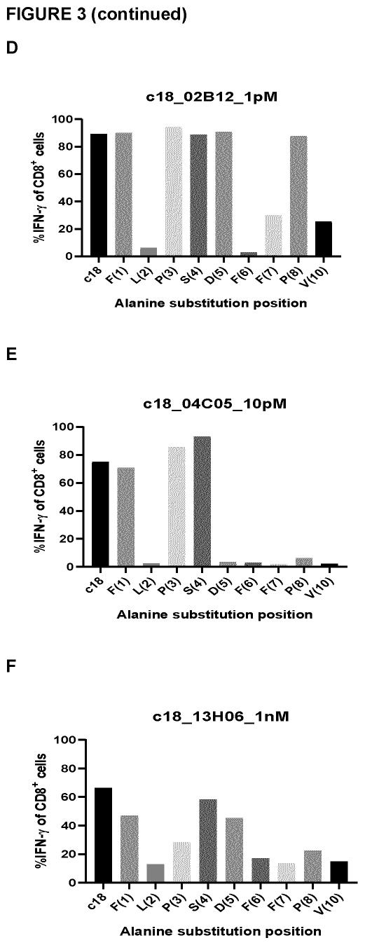

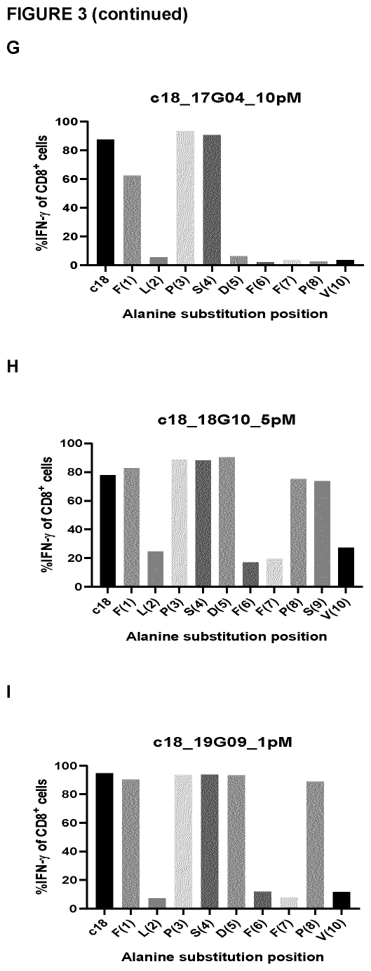

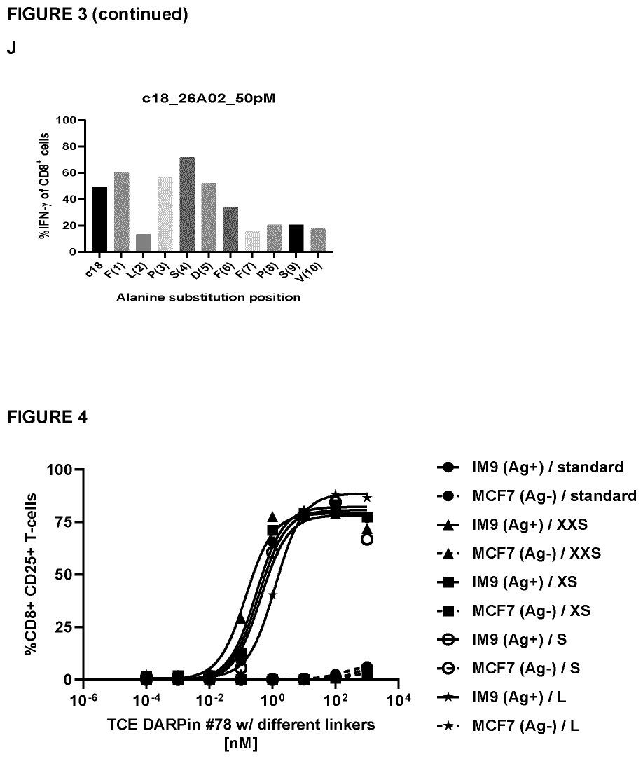

The inventors of the present invention have discovered that a surprisingly large number of peptide residues are important for the specific interaction between binding proteins of the invention comprising an ankyrin repeat domain with binding specificity for HBVc18pMHC and the HBVc18 target peptide in the HBVc18pMHC complex. For instance, in the appended examples, several peptide residues that are important for the specific interaction between binding proteins of the invention comprising an ankyrin repeat domain with binding specificity for HBVc18pMHC and the HBVc18 target peptide in the HBVc18pMHC complex have been identified (see FIGS. 3A-J). The inventors theorize that this finding may reflect a structural difference between the binding surface formed by the designed ankyrin repeat domains of the present invention and that formed by other binding proteins, such as antibodies and T cell receptors (TCRs). Without being bound by any theory, it is believed that the higher the number of amino acid residues involved in the binding of a repeat domain and its target peptide, the higher is the binding specificity.

In one embodiment, said target peptide is selected from the group consisting of (i) a peptide derived from a protein expressed in a tumor cell, (ii) a peptide derived from a protein of an infectious agent, such as a bacterial infectious agent or a viral infectious agent, preferably a viral infectious agent, and (iii) a peptide derived from a protein associated with an autoimmune disorder. In one embodiment, said target peptide is derived from a protein of a viral infectious agent, preferably a virus-specific protein. In one embodiment, said target peptide is derived from HBcAg. In one embodiment, said target peptide has the amino acid sequence of SEQ ID NO: 51.

In one embodiment, said MHC is MHC class I. In one embodiment, said MHC class I is HLA-A*02. In one embodiment, said HLA-A*02 is HLA-A*0201. In one embodiment, said HLA-A*0201 has the amino acid sequence of SEQ ID NO: 69. Alternatively, in another embodiment, said MHC is not HLA-A*02.

In another aspect, the invention relates to a recombinant binding protein comprising a designed repeat domain obtainable by one of the methods of the invention as described herein.

In another aspect, the invention relates to a recombinant binding protein comprising a first designed repeat domain, wherein said first repeat domain has binding specificity for a first target peptide-MHC complex.

In a preferred embodiment, the binding protein of the invention comprises a first designed repeat domain with binding specificity for a first target peptide-MHC complex, wherein said first target peptide is selected from the group consisting of (i) a peptide derived from a protein expressed in a tumor cell, (ii) a peptide derived from a protein of an infectious agent, such as a bacterial infectious agent or a viral infectious agent, preferably a viral infectious agent, and (iii) a peptide derived from a protein associated with an autoimmune disorder.

In one embodiment, said first target peptide is derived from a protein of a viral infectious agent, preferably a virus-specific protein. In one embodiment, said first target peptide is derived from a virus-specific protein. In one embodiment, said first target peptide is derived from HBcAg. In one embodiment, said first target peptide has the amino acid sequence of SEQ ID NO: 51.

In a preferred embodiment, the first MHC is MHC class I. In one embodiment, said first MHC class I is HLA-A*02. In one embodiment, said HLA-A*02 is HLA-A*0201. In one embodiment, said HLA-A*0201 has the amino acid sequence of SEQ ID NO: 69. Alternatively, in another embodiment, said first MHC is not HLA-A*02.

In one embodiment, said first repeat domain binds to said first target peptide-MHC complex in PBS with a dissociation constant (K_{D}) below 10⁻⁷M.

In one embodiment, said binding of said first repeat domain to said first target peptide-MHC complex comprises interaction of said first repeat domain with at least one, at least two, at least three, at least four, at least five, at least six, or at least seven amino acid residues of said first target peptide. Thus, in one embodiment, binding of said first repeat domain to said first target peptide-MHC complex comprises interaction of said first repeat domain with at least one amino acid residue of said first target peptide. In one embodiment, binding of said first repeat domain to said first target peptide-MHC complex comprises interaction of said first repeat domain with at least two amino acid residues of said first target peptide. In one embodiment, binding of said first repeat domain to said first target peptide-MHC complex comprises interaction of said first repeat domain with at least three amino acid residues of said first target peptide. In one embodiment, binding of said first repeat domain to said first target peptide-MHC complex comprises interaction of said first repeat domain with at least four amino acid residues of said first target peptide. In one embodiment, binding of said first repeat domain to said first target peptide-MHC complex comprises interaction of said first repeat domain with at least five amino acid residues of said first target peptide. In one embodiment, binding of said first repeat domain to said first target peptide-MHC complex comprises interaction of said first repeat domain with at least six amino acid residues of said first target peptide. In one embodiment, binding of said first repeat domain to said first target peptide-MHC complex comprises interaction of said first repeat domain with at least seven amino acid residues of said first target peptide. In a further or alternative embodiment, binding of said first repeat domain to said first target peptide-MHC complex comprises interaction of said first repeat domain with at least one amino acid residue of said first MHC.

In a preferred embodiment, said first designed repeat domain is a designed ankyrin repeat domain. In a particularly preferred embodiment, said first designed ankyrin repeat domain is a designed ankyrin repeat domain with binding specificity for a target peptide-MHC complex as described more specifically in any of the aspects or embodiments herein.

In one embodiment, a designed ankyrin repeat domain with binding specificity for a target peptide-MHC complex according to the present invention comprises an ankyrin repeat module.

In one particular embodiment, a designed ankyrin repeat domain with binding specificity for a target peptide-MHC complex according to the present invention comprises an ankyrin repeat module comprising an amino acid sequence selected from the group consisting of (1) SEQ ID NOs: 27 to 50 and (2) sequences in which up to 9, or up to 8, or up to 7, or up to 6, or up to 5, or up to 4, or up to 3, or up to 2, or up to 1 amino acids in any of SEQ ID NOs: 27 to 50 are substituted by another amino acid. Thus, in one embodiment, said ankyrin repeat module comprises an amino acid sequence selected from the group consisting of (1) SEQ ID NOs: 27 to 50 and (2) sequences in which up to 9 amino acids in any of SEQ ID NOs: 27 to 50 are substituted by another amino acid. Thus, in one embodiment, said ankyrin repeat module comprises an amino acid sequence selected from the group consisting of (1) SEQ ID NOs: 27 to 50 and (2) sequences in which up to 3 amino acids in any of SEQ ID NOs: 27 to 50 are substituted by another amino acid. In one embodiment, said ankyrin repeat module comprises an amino acid sequence selected from the group consisting of (1) SEQ ID NOs: 27 to 50 and (2) sequences in which up to 2 amino acids in any of SEQ ID NOs: 27 to 50 are substituted by another amino acid. In one embodiment, said ankyrin repeat module comprises an amino acid sequence selected from the group consisting of (1) SEQ ID NOs: 27 to 50 and (2) sequences in which up to 1 amino acid in any of SEQ ID NOs: 27 to 50 is substituted by another amino acid. In one embodiment, said ankyrin repeat module comprises an amino acid sequence selected from the group consisting of SEQ ID NOs: 27 to 50. In one preferred embodiment, said target peptide has the amino acid sequence of SEQ ID NO: 51.

In one particular embodiment, a designed ankyrin repeat domain with binding specificity for a target peptide-MHC complex according to the present invention comprises an ankyrin repeat module comprising an amino acid sequence selected from the group consisting of (1) SEQ ID NOs: 29, 30, 37 to 43 and 48 to 50 and (2) sequences in which up to 9, or up to 8, or up to 7, or up to 6, or up to 5, or up to 4, or up to 3, or up to 2, or up to 1 amino acids in any of SEQ ID NOs: 29, 30, 37 to 43 and 48 to 50 are substituted by another amino acid. Thus, in one embodiment, said ankyrin repeat module comprises an amino acid sequence selected from the group consisting of (1) SEQ ID NOs: 29, 30, 37 to 43 and 48 to 50 and (2) sequences in which up to 3 amino acids in any of SEQ ID NOs: 29, 30, 37 to 43 and 48 to 50 are substituted by another amino acid. In one embodiment, said ankyrin repeat module comprises an amino acid sequence selected from the group consisting of (1) SEQ ID NOs: 29, 30, 37 to 43 and 48 to 50 and (2) sequences in which up to 2 amino acids in any of SEQ ID NOs: 29, 30, 37 to 43 and 48 to 50 are substituted by another amino acid. In one embodiment, said ankyrin repeat module comprises an amino acid sequence selected from the group consisting of (1) SEQ ID NOs: 29, 30, 37 to 43 and 48 to 50 and (2) sequences in which up to 1 amino acid in any of SEQ ID NOs: 29, 30, 37 to 43 and 48 to 50 is substituted by another amino acid. In one embodiment, said ankyrin repeat module comprises an amino acid sequence selected from the group consisting of SEQ ID NOs: 29, 30, 37 to 43 and 48 to 50. In one preferred embodiment, said target peptide has the amino acid sequence of SEQ ID NO: 51.

In one particular embodiment, a designed ankyrin repeat domain with binding specificity for a target peptide-MHC complex according to the present invention comprises an ankyrin repeat module comprising an amino acid sequence selected from the group consisting of (1) SEQ ID NOs: 37, 38, 42 and 43 and (2) sequences in which up to 9, or up to 8, or up to 7, or up to 6, or up to 5, or up to 4, or up to 3, or up to 2, or up to 1 amino acids in any of SEQ ID NOs: 37, 38, 42 and 43 are substituted by another amino acid. Thus, in one embodiment, said ankyrin repeat module comprises an amino acid sequence selected from the group consisting of (1) SEQ ID NOs: 37, 38, 42 and 43 and (2) sequences in which up to 3 amino acids in any of SEQ ID NOs: 37, 38, 42 and 43 are substituted by another amino acid. In one embodiment, said ankyrin repeat module comprises an amino acid sequence selected from the group consisting of (1) SEQ ID NOs: 37, 38, 42 and 43 and (2) sequences in which up to 2 amino acids in any of SEQ ID NOs: 37, 38, 42 and 43 are substituted by another amino acid. In one embodiment, said ankyrin repeat module comprises an amino acid sequence selected from the group consisting of (1) SEQ ID NOs: 37, 38, 42 and 43 and (2) sequences in which up to 1 amino acid in any of SEQ ID NOs: 37, 38, 42 and 43 is substituted by another amino acid. In one embodiment, said ankyrin repeat module comprises an amino acid sequence selected from the group consisting of SEQ ID NOs: 37, 38, 42 and 43. In one preferred embodiment, said target peptide has the amino acid sequence of SEQ ID NO: 51.

In one embodiment, a designed ankyrin repeat domain with binding specificity for a target peptide-MHC complex according to the present invention comprises a first ankyrin repeat module and a second ankyrin repeat module. In one embodiment, said first ankyrin repeat module is located N-terminally of said second ankyrin repeat module within said ankyrin repeat domain.

In one embodiment, a designed ankyrin repeat domain with binding specificity for a target peptide-MHC complex according to the present invention comprises a first ankyrin repeat module and a second ankyrin repeat module and a third ankyrin repeat module. In one embodiment, said first ankyrin repeat module is located N-terminally of said second ankyrin repeat module within said ankyrin repeat domain, and said second ankyrin repeat module is located N-terminally of said third ankyrin repeat module within said ankyrin repeat domain.

In one particular embodiment, a designed ankyrin repeat domain with binding specificity for a target peptide-MHC complex according to the present invention comprises a first, a second and optionally a third ankyrin repeat module, wherein each of said first, said second and, if present, said third ankyrin repeat module independently comprises an amino acid sequence selected from the group consisting of (1) SEQ ID NOs: 27 to 50 and (2) sequences in which up to 9, or up to 8, or up to 7, or up to 6, or up to 5, or up to 4, or up to 3, or up to 2, or up to 1 amino acids in any of SEQ ID NOs: 27 to 50 are substituted by another amino acid. In one embodiment, each of said first, said second and, if present, said third ankyrin repeat module independently comprises an amino acid sequence selected from the group consisting of (1) SEQ ID NOs: 29, 30, 37 to 43 and 48 to 50 and (2) sequences in which up to 9, or up to 8, or up to 7, or up to 6, or up to 5, or up to 4, or up to 3, or up to 2, or up to 1 amino acids in any of SEQ ID NOs: 29, 30, 37 to 43 and 48 to 50 are substituted by another amino acid. In one embodiment, each of said first, said second and, if present, said third ankyrin repeat module independently comprises an amino acid sequence selected from the group consisting of (1) SEQ ID NOs: 37, 38, 42 and 43 and (2) sequences in which up to 9, or up to 8, or up to 7, or up to 6, or up to 5, or up to 4, or up to 3, or up to 2, or up to 1 amino acids in any of SEQ ID NOs: 37, 38, 42 and 43 are substituted by another amino acid. In one preferred embodiment, said target peptide has the amino acid sequence of SEQ ID NO: 51.

In one particular embodiment, a designed ankyrin repeat domain with binding specificity for a target peptide-MHC complex according to the present invention comprises a first ankyrin repeat module and a second ankyrin repeat module, wherein said first ankyrin repeat module comprises an amino acid sequence selected from the group consisting of (1) SEQ ID NO: 37 and (2) sequences in which up to 9, or up to 8, or up to 7, or up to 6, or up to 5, or up to 4, or up to 3, or up to 2, or up to 1 amino acids in SEQ ID NO: 37 are substituted by another amino acid, and said second ankyrin repeat module comprises an amino acid sequence selected from the group consisting of (1) SEQ ID NO: 38 and (2) sequences in which up to 9, or up to 8, or up to 7, or up to 6, or up to 5, or up to 4, or up to 3, or up to 2, or up to 1 amino acids of SEQ ID NO: 38 are substituted by another amino acid. In one embodiment, said first ankyrin repeat module is located N-terminally of said second ankyrin repeat module within said ankyrin repeat domain. In one preferred embodiment, said target peptide has the amino acid sequence of SEQ ID NO: 51.

In one embodiment, in such an ankyrin repeat domain said first ankyrin repeat module comprises an amino acid sequence selected from the group consisting of (1) SEQ ID NO: 37 and (2) sequences in which up to 6 amino acids in SEQ ID NO: 37 are substituted by another amino acid, and said second ankyrin repeat module comprises an amino acid sequence selected from the group consisting of (1) SEQ ID NO: 38 and (2) sequences in which up to 6 amino acids of SEQ ID NO: 38 are substituted by another amino acid. In one embodiment, in such an ankyrin repeat domain said first ankyrin repeat module comprises an amino acid sequence selected from the group consisting of (1) SEQ ID NO: 37 and (2) sequences in which up to 5 amino acids in SEQ ID NO: 37 are substituted by another amino acid, and said second ankyrin repeat module comprises an amino acid sequence selected from the group consisting of (1) SEQ ID NO: 38 and (2) sequences in which up to 5 amino acids of SEQ ID NO: 38 are substituted by another amino acid. In one embodiment, in such an ankyrin repeat domain said first ankyrin repeat module comprises an amino acid sequence selected from the group consisting of (1) SEQ ID NO: 37 and (2) sequences in which up to 4 amino acids in SEQ ID NO: 37 are substituted by another amino acid, and said second ankyrin repeat module comprises an amino acid sequence selected from the group consisting of (1) SEQ ID NO: 38 and (2) sequences in which up to 4 amino acids of SEQ ID NO: 38 are substituted by another amino acid. In one embodiment, in such an ankyrin repeat domain said first ankyrin repeat module comprises an amino acid sequence selected from the group consisting of (1) SEQ ID NO: 37 and (2) sequences in which up to 3 amino acids in SEQ ID NO: 37 are substituted by another amino acid, and said second ankyrin repeat module comprises an amino acid sequence selected from the group consisting of (1) SEQ ID NO: 38 and (2) sequences in which up to 3 amino acids of SEQ ID NO: 38 are substituted by another amino acid. In one embodiment, in such an ankyrin repeat domain said first ankyrin repeat module comprises an amino acid sequence selected from the group consisting of (1) SEQ ID NO: 37 and (2) sequences in which up to 2 amino acids in SEQ ID NO: 37 are substituted by another amino acid, and said second ankyrin repeat module comprises an amino acid sequence selected from the group consisting of (1) SEQ ID NO: 38 and (2) sequences in which up to 2 amino acids of SEQ ID NO: 38 are substituted by another amino acid. In one embodiment, in such an ankyrin repeat domain said first ankyrin repeat module comprises an amino acid sequence selected from the group consisting of (1) SEQ ID NO: 37 and (2) sequences in which 1 amino acid in SEQ ID NO: 37 is substituted by another amino acid, and said second ankyrin repeat module comprises an amino acid sequence selected from the group consisting of (1) SEQ ID NO: 38 and (2) sequences in which 1 amino acid of SEQ ID NO: 38 is substituted by another amino acid. In one embodiment, in such an ankyrin repeat domain said first ankyrin repeat module comprises the amino acid sequence of SEQ ID NO: 37, and said second ankyrin repeat module comprises the amino acid sequence of SEQ ID NO: 38. In one embodiment, said first ankyrin repeat module is located N-terminally of said second ankyrin repeat module within said ankyrin repeat domain. In one preferred embodiment, said target peptide has the amino acid sequence of SEQ ID NO: 51.

In one particular embodiment, a designed ankyrin repeat domain with binding specificity for a target peptide-MHC complex according to the present invention comprises a first ankyrin repeat module and a second ankyrin repeat module, wherein said first ankyrin repeat module comprises an amino acid sequence selected from the group consisting of (1) SEQ ID NO: 42 and (2) sequences in which up to 9, or up to 8, or up to 7, or up to 6, or up to 5, or up to 4, or up to 3, or up to 2, or up to 1 amino acids in SEQ ID NO: 42 are substituted by another amino acid, and said second ankyrin repeat module comprises an amino acid sequence selected from the group consisting of (1) SEQ ID NO: 43 and (2) sequences in which up to 9, or up to 8, or up to 7, or up to 6, or up to 5, or up to 4, or up to 3, or up to 2, or up to 1 amino acids of SEQ ID NO: 43 are substituted by another amino acid. In one embodiment, said first ankyrin repeat module is located N-terminally of said second ankyrin repeat module within said ankyrin repeat domain. In one preferred embodiment, said target peptide has the amino acid sequence of SEQ ID NO: 51.

In one embodiment, in such an ankyrin repeat domain said first ankyrin repeat module comprises an amino acid sequence selected from the group consisting of (1) SEQ ID NO: 42 and (2) sequences in which up to 6 amino acids in SEQ ID NO: 42 are substituted by another amino acid, and said second ankyrin repeat module comprises an amino acid sequence selected from the group consisting of (1) SEQ ID NO: 43 and (2) sequences in which up to 6 amino acids of SEQ ID NO: 43 are substituted by another amino acid. In one embodiment, in such an ankyrin repeat domain said first ankyrin repeat module comprises an amino acid sequence selected from the group consisting of (1) SEQ ID NO: 42 and (2) sequences in which up to 5 amino acids in SEQ ID NO: 42 are substituted by another amino acid, and said second ankyrin repeat module comprises an amino acid sequence selected from the group consisting of (1) SEQ ID NO: 43 and (2) sequences in which up to 5 amino acids of SEQ ID NO: 43 are substituted by another amino acid. In one embodiment, in such an ankyrin repeat domain said first ankyrin repeat module comprises an amino acid sequence selected from the group consisting of (1) SEQ ID NO: 42 and (2) sequences in which up to 4 amino acids in SEQ ID NO: 42 are substituted by another amino acid, and said second ankyrin repeat module comprises an amino acid sequence selected from the group consisting of (1) SEQ ID NO: 43 and (2) sequences in which up to 4 amino acids of SEQ ID NO: 43 are substituted by another amino acid. In one embodiment, in such an ankyrin repeat domain said first ankyrin repeat module comprises an amino acid sequence selected from the group consisting of (1) SEQ ID NO: 42 and (2) sequences in which up to 3 amino acids in SEQ ID NO: 42 are substituted by another amino acid, and said second ankyrin repeat module comprises an amino acid sequence selected from the group consisting of (1) SEQ ID NO: 43 and (2) sequences in which up to 3 amino acids of SEQ ID NO: 43 are substituted by another amino acid. In one embodiment, in such an ankyrin repeat domain said first ankyrin repeat module comprises an amino acid sequence selected from the group consisting of (1) SEQ ID NO: 42 and (2) sequences in which up to 2 amino acids in SEQ ID NO: 42 are substituted by another amino acid, and said second ankyrin repeat module comprises an amino acid sequence selected from the group consisting of (1) SEQ ID NO: 43 and (2) sequences in which up to 2 amino acids of SEQ ID NO: 43 are substituted by another amino acid. In one embodiment, in such an ankyrin repeat domain said first ankyrin repeat module comprises an amino acid sequence selected from the group consisting of (1) SEQ ID NO: 42 and (2) sequences in which 1 amino acid in SEQ ID NO: 42 is substituted by another amino acid, and said second ankyrin repeat module comprises an amino acid sequence selected from the group consisting of (1) SEQ ID NO: 43 and (2) sequences in which 1 amino acid of SEQ ID NO: 43 is substituted by another amino acid. In one embodiment, in such an ankyrin repeat domain said first ankyrin repeat module comprises the amino acid sequence of SEQ ID NO: 42, and said second ankyrin repeat module comprises the amino acid sequence of SEQ ID NO: 43. In one embodiment, said first ankyrin repeat module is located N-terminally of said second ankyrin repeat module within said ankyrin repeat domain. In one preferred embodiment, said target peptide has the amino acid sequence of SEQ ID NO: 51.

In one particular embodiment, a designed ankyrin repeat domain with binding specificity for a target peptide-MHC complex according to the present invention comprises a first ankyrin repeat module and a second ankyrin repeat module, wherein said first ankyrin repeat module comprises an amino acid sequence selected from the group consisting of (1) SEQ ID NO: 29 and (2) sequences in which up to 9, or up to 8, or up to 7, or up to 6, or up to 5, or up to 4, or up to 3, or up to 2, or up to 1 amino acids in SEQ ID NO: 29 are substituted by another amino acid, and said second ankyrin repeat module comprises an amino acid sequence selected from the group consisting of (1) SEQ ID NO: 30 and (2) sequences in which up to 9, or up to 8, or up to 7, or up to 6, or up to 5, or up to 4, or up to 3, or up to 2, or up to 1 amino acids of SEQ ID NO: 30 are substituted by another amino acid. In one embodiment, said first ankyrin repeat module is located N-terminally of said second ankyrin repeat module within said ankyrin repeat domain. In one preferred embodiment, said target peptide has the amino acid sequence of SEQ ID NO: 51.

In one embodiment, in such an ankyrin repeat domain said first ankyrin repeat module comprises an amino acid sequence selected from the group consisting of (1) SEQ ID NO: 29 and (2) sequences in which up to 6 amino acids in SEQ ID NO: 29 are substituted by another amino acid, and said second ankyrin repeat module comprises an amino acid sequence selected from the group consisting of (1) SEQ ID NO: 30 and (2) sequences in which up to 6 amino acids of SEQ ID NO: 30 are substituted by another amino acid. In one embodiment, in such an ankyrin repeat domain said first ankyrin repeat module comprises an amino acid sequence selected from the group consisting of (1) SEQ ID NO: 29 and (2) sequences in which up to 5 amino acids in SEQ ID NO: 29 are substituted by another amino acid, and said second ankyrin repeat module comprises an amino acid sequence selected from the group consisting of (1) SEQ ID NO: 30 and (2) sequences in which up to 5 amino acids of SEQ ID NO: 30 are substituted by another amino acid. In one embodiment, in such an ankyrin repeat domain said first ankyrin repeat module comprises an amino acid sequence selected from the group consisting of (1) SEQ ID NO: 29 and (2) sequences in which up to 4 amino acids in SEQ ID NO: 29 are substituted by another amino acid, and said second ankyrin repeat module comprises an amino acid sequence selected from the group consisting of (1) SEQ ID NO: 30 and (2) sequences in which up to 4 amino acids of SEQ ID NO: 30 are substituted by another amino acid. In one embodiment, in such an ankyrin repeat domain said first ankyrin repeat module comprises an amino acid sequence selected from the group consisting of (1) SEQ ID NO: 29 and (2) sequences in which up to 3 amino acids in SEQ ID NO: 29 are substituted by another amino acid, and said second ankyrin repeat module comprises an amino acid sequence selected from the group consisting of (1) SEQ ID NO: 30 and (2) sequences in which up to 3 amino acids of SEQ ID NO: 30 are substituted by another amino acid. In one embodiment, in such an ankyrin repeat domain said first ankyrin repeat module comprises an amino acid sequence selected from the group consisting of (1) SEQ ID NO: 29 and (2) sequences in which up to 2 amino acids in SEQ ID NO: 29 are substituted by another amino acid, and said second ankyrin repeat module comprises an amino acid sequence selected from the group consisting of (1) SEQ ID NO: 30 and (2) sequences in which up to 2 amino acids of SEQ ID NO: 30 are substituted by another amino acid. In one embodiment, in such an ankyrin repeat domain said first ankyrin repeat module comprises an amino acid sequence selected from the group consisting of (1) SEQ ID NO: 29 and (2) sequences in which 1 amino acid in SEQ ID NO: 29 is substituted by another amino acid, and said second ankyrin repeat module comprises an amino acid sequence selected from the group consisting of (1) SEQ ID NO: 30 and (2) sequences in which 1 amino acid of SEQ ID NO: 30 is substituted by another amino acid. In one embodiment, in such an ankyrin repeat domain said first ankyrin repeat module comprises the amino acid sequence of SEQ ID NO: 29, and said second ankyrin repeat module comprises the amino acid sequence of SEQ ID NO: 30. In one embodiment, said first ankyrin repeat module is located N-terminally of said second ankyrin repeat module within said ankyrin repeat domain. In one preferred embodiment, said target peptide has the amino acid sequence of SEQ ID NO: 51.

In one particular embodiment, a designed ankyrin repeat domain with binding specificity for a target peptide-MHC complex according to the present invention comprises a first ankyrin repeat module and a second ankyrin repeat module and a third ankyrin repeat module, wherein said first ankyrin repeat module comprises an amino acid sequence selected from the group consisting of (1) SEQ ID NO: 39 and (2) sequences in which up to 9, or up to 8, or up to 7, or up to 6, or up to 5, or up to 4, or up to 3, or up to 2, or up to 1 amino acids in SEQ ID NO: 39 are substituted by another amino acid, and said second ankyrin repeat module comprises an amino acid sequence selected from the group consisting of (1) SEQ ID NO: 40 and (2) sequences in which up to 9, or up to 8, or up to 7, or up to 6, or up to 5, or up to 4, or up to 3, or up to 2, or up to 1 amino acids of SEQ ID NO: 40 are substituted by another amino acid, and said third ankyrin repeat module comprises an amino acid sequence selected from the group consisting of (1) SEQ ID NO: 41 and (2) sequences in which up to 9, or up to 8, or up to 7, or up to 6, or up to 5, or up to 4, or up to 3, or up to 2, or up to 1 amino acids of SEQ ID NO: 41 are substituted by another amino acid. In one embodiment, said first ankyrin repeat module is located N-terminally of said second ankyrin repeat module within said ankyrin repeat domain, and said second ankyrin repeat module is located N-terminally of said third ankyrin repeat module within said ankyrin repeat domain. In one preferred embodiment, said target peptide has the amino acid sequence of SEQ ID NO: 51.

In one embodiment, in such an ankyrin repeat domain said first ankyrin repeat module comprises an amino acid sequence selected from the group consisting of (1) SEQ ID NO: 39 and (2) sequences in which up to 6 amino acids in SEQ ID NO: 39 are substituted by another amino acid, and said second ankyrin repeat module comprises an amino acid sequence selected from the group consisting of (1) SEQ ID NO: 40 and (2) sequences in which up to 6 amino acids of SEQ ID NO: 40 are substituted by another amino acid, and said third ankyrin repeat module comprises an amino acid sequence selected from the group consisting of (1) SEQ ID NO: 41 and (2) sequences in which up to 6 amino acids of SEQ ID NO: 41 are substituted by another amino acid. In one embodiment, in such an ankyrin repeat domain said first ankyrin repeat module comprises an amino acid sequence selected from the group consisting of (1) SEQ ID NO: 39 and (2) sequences in which up to 5 amino acids in SEQ ID NO: 39 are substituted by another amino acid, and said second ankyrin repeat module comprises an amino acid sequence selected from the group consisting of (1) SEQ ID NO: 40 and (2) sequences in which up to 5 amino acids of SEQ ID NO: 40 are substituted by another amino acid, and said third ankyrin repeat module comprises an amino acid sequence selected from the group consisting of (1) SEQ ID NO: 41 and (2) sequences in which up to 5 amino acids of SEQ ID NO: 41 are substituted by another amino acid. In one embodiment, in such an ankyrin repeat domain said first ankyrin repeat module comprises an amino acid sequence selected from the group consisting of (1) SEQ ID NO: 39 and (2) sequences in which up to 4 amino acids in SEQ ID NO: 39 are substituted by another amino acid, and said second ankyrin repeat module comprises an amino acid sequence selected from the group consisting of (1) SEQ ID NO: 40 and (2) sequences in which up to 4 amino acids of SEQ ID NO: 40 are substituted by another amino acid, and said third ankyrin repeat module comprises an amino acid sequence selected from the group consisting of (1) SEQ ID NO: 41 and (2) sequences in which up to 4 amino acids of SEQ ID NO: 41 are substituted by another amino acid. In one embodiment, in such an ankyrin repeat domain said first ankyrin repeat module comprises an amino acid sequence selected from the group consisting of (1) SEQ ID NO: 39 and (2) sequences in which up to 3 amino acids in SEQ ID NO: 39 are substituted by another amino acid, and said second ankyrin repeat module comprises an amino acid sequence selected from the group consisting of (1) SEQ ID NO: 40 and (2) sequences in which up to 3 amino acids of SEQ ID NO: 40 are substituted by another amino acid, and said third ankyrin repeat module comprises an amino acid sequence selected from the group consisting of (1) SEQ ID NO: 41 and (2) sequences in which up to 3 amino acids of SEQ ID NO: 41 are substituted by another amino acid. In one embodiment, in such an ankyrin repeat domain said first ankyrin repeat module comprises an amino acid sequence selected from the group consisting of (1) SEQ ID NO: 39 and (2) sequences in which up to 2 amino acids in SEQ ID NO: 39 are substituted by another amino acid, and said second ankyrin repeat module comprises an amino acid sequence selected from the group consisting of (1) SEQ ID NO: 40 and (2) sequences in which up to 2 amino acids of SEQ ID NO: 40 are substituted by another amino acid, and said third ankyrin repeat module comprises an amino acid sequence selected from the group consisting of (1) SEQ ID NO: 41 and (2) sequences in which up to 2 amino acids of SEQ ID NO: 41 are substituted by another amino acid. In one embodiment, in such an ankyrin repeat domain said first ankyrin repeat module comprises an amino acid sequence selected from the group consisting of (1) SEQ ID NO: 39 and (2) sequences in which 1 amino acid in SEQ ID NO: 39 is substituted by another amino acid, and said second ankyrin repeat module comprises an amino acid sequence selected from the group consisting of (1) SEQ ID NO: 40 and (2) sequences in which 1 amino acid of SEQ ID NO: 40 is substituted by another amino acid, and said third ankyrin repeat module comprises an amino acid sequence selected from the group consisting of (1) SEQ ID NO: 41 and (2) sequences in which 1 amino acid of SEQ ID NO: 41 is substituted by another amino acid. In one embodiment, in such an ankyrin repeat domain said first ankyrin repeat module comprises the amino acid sequence of SEQ ID NO: 39, and said second ankyrin repeat module comprises the amino acid sequence of SEQ ID NO: 40, and said third ankyrin repeat module comprises the amino acid sequence of SEQ ID NO: 41. In one embodiment, said first ankyrin repeat module is located N-terminally of said second ankyrin repeat module within said ankyrin repeat domain, and said second ankyrin repeat module is located N-terminally of said third ankyrin repeat module within said ankyrin repeat domain. In one preferred embodiment, said target peptide has the amino acid sequence of SEQ ID NO: 51.

In one particular embodiment, a designed ankyrin repeat domain with binding specificity for a target peptide-MHC complex according to the present invention comprises a first ankyrin repeat module and a second ankyrin repeat module and a third ankyrin repeat module, wherein said first ankyrin repeat module comprises an amino acid sequence selected from the group consisting of (1) SEQ ID NO: 48 and (2) sequences in which up to 9, or up to 8, or up to 7, or up to 6, or up to 5, or up to 4, or up to 3, or up to 2, or up to 1 amino acids in SEQ ID NO: 48 are substituted by another amino acid, and said second ankyrin repeat module comprises an amino acid sequence selected from the group consisting of (1) SEQ ID NO: 49 and (2) sequences in which up to 9, or up to 8, or up to 7, or up to 6, or up to 5, or up to 4, or up to 3, or up to 2, or up to 1 amino acids of SEQ ID NO: 49 are substituted by another amino acid, and said third ankyrin repeat module comprises an amino acid sequence selected from the group consisting of (1) SEQ ID NO: 50 and (2) sequences in which up to 9, or up to 8, or up to 7, or up to 6, or up to 5, or up to 4, or up to 3, or up to 2, or up to 1 amino acids of SEQ ID NO: 50 are substituted by another amino acid. In one embodiment, said first ankyrin repeat module is located N-terminally of said second ankyrin repeat module within said ankyrin repeat domain, and said second ankyrin repeat module is located N-terminally of said third ankyrin repeat module within said ankyrin repeat domain. In one preferred embodiment, said target peptide has the amino acid sequence of SEQ ID NO: 51.

In one embodiment, in such an ankyrin repeat domain said first ankyrin repeat module comprises an amino acid sequence selected from the group consisting of (1) SEQ ID NO: 48 and (2) sequences in which up to 6 amino acids in SEQ ID NO: 48 are substituted by another amino acid, and said second ankyrin repeat module comprises an amino acid sequence selected from the group consisting of (1) SEQ ID NO: 49 and (2) sequences in which up to 6 amino acids of SEQ ID NO: 49 are substituted by another amino acid, and said third ankyrin repeat module comprises an amino acid sequence selected from the group consisting of (1) SEQ ID NO: 50 and (2) sequences in which up to 6 amino acids of SEQ ID NO: 50 are substituted by another amino acid. In one embodiment, in such an ankyrin repeat domain said first ankyrin repeat module comprises an amino acid sequence selected from the group consisting of (1) SEQ ID NO: 48 and (2) sequences in which up to 5 amino acids in SEQ ID NO: 48 are substituted by another amino acid, and said second ankyrin repeat module comprises an amino acid sequence selected from the group consisting of (1) SEQ ID NO: 49 and (2) sequences in which up to 5 amino acids of SEQ ID NO: 49 are substituted by another amino acid, and said third ankyrin repeat module comprises an amino acid sequence selected from the group consisting of (1) SEQ ID NO: 50 and (2) sequences in which up to 5 amino acids of SEQ ID NO: 50 are substituted by another amino acid. In one embodiment, in such an ankyrin repeat domain said first ankyrin repeat module comprises an amino acid sequence selected from the group consisting of (1) SEQ ID NO: 48 and (2) sequences in which up to 4 amino acids in SEQ ID NO: 48 are substituted by another amino acid, and said second ankyrin repeat module comprises an amino acid sequence selected from the group consisting of (1) SEQ ID NO: 49 and (2) sequences in which up to 4 amino acids of SEQ ID NO: 49 are substituted by another amino acid, and said third ankyrin repeat module comprises an amino acid sequence selected from the group consisting of (1) SEQ ID NO: 50 and (2) sequences in which up to 4 amino acids of SEQ ID NO: 50 are substituted by another amino acid. In one embodiment, in such an ankyrin repeat domain said first ankyrin repeat module comprises an amino acid sequence selected from the group consisting of (1) SEQ ID NO: 48 and (2) sequences in which up to 3 amino acids in SEQ ID NO: 48 are substituted by another amino acid, and said second ankyrin repeat module comprises an amino acid sequence selected from the group consisting of (1) SEQ ID NO: 49 and (2) sequences in which up to 3 amino acids of SEQ ID NO: 49 are substituted by another amino acid, and said third ankyrin repeat module comprises an amino acid sequence selected from the group consisting of (1) SEQ ID NO: 50 and (2) sequences in which up to 3 amino acids of SEQ ID NO: 50 are substituted by another amino acid. In one embodiment, in such an ankyrin repeat domain said first ankyrin repeat module comprises an amino acid sequence selected from the group consisting of (1) SEQ ID NO: 48 and (2) sequences in which up to 2 amino acids in SEQ ID NO: 48 are substituted by another amino acid, and said second ankyrin repeat module comprises an amino acid sequence selected from the group consisting of (1) SEQ ID NO: 49 and (2) sequences in which up to 2 amino acids of SEQ ID NO: 49 are substituted by another amino acid, and said third ankyrin repeat module comprises an amino acid sequence selected from the group consisting of (1) SEQ ID NO: 50 and (2) sequences in which up to 2 amino acids of SEQ ID NO: 50 are substituted by another amino acid. In one embodiment, in such an ankyrin repeat domain said first ankyrin repeat module comprises an amino acid sequence selected from the group consisting of (1) SEQ ID NO: 48 and (2) sequences in which 1 amino acid in SEQ ID NO: 48 is substituted by another amino acid, and said second ankyrin repeat module comprises an amino acid sequence selected from the group consisting of (1) SEQ ID NO: 49 and (2) sequences in which 1 amino acid of SEQ ID NO: 49 is substituted by another amino acid, and said third ankyrin repeat module comprises an amino acid sequence selected from the group consisting of (1) SEQ ID NO: 50 and (2) sequences in which 1 amino acid of SEQ ID NO: 50 is substituted by another amino acid. In one embodiment, in such an ankyrin repeat domain said first ankyrin repeat module comprises the amino acid sequence of SEQ ID NO: 48, and said second ankyrin repeat module comprises the amino acid sequence of SEQ ID NO: 49, and said third ankyrin repeat module comprises the amino acid sequence of SEQ ID NO: 50. In one embodiment, said first ankyrin repeat module is located N-terminally of said second ankyrin repeat module within said ankyrin repeat domain, and said second ankyrin repeat module is located N-terminally of said third ankyrin repeat module within said ankyrin repeat domain. In one preferred embodiment, said target peptide has the amino acid sequence of SEQ ID NO: 51.

In one preferred embodiment, all of said amino acid substitutions of said ankyrin repeat module(s) as described and referred to herein occur in framework positions of said ankyrin repeat module(s), wherein typically the overall structure of the module(s) is not affected by the substitutions.

In one preferred embodiment, all of said amino acid substitutions of said ankyrin repeat module(s) as described and referred to herein occur in positions other than the randomized positions 3, 4, 6, 14 and 15 of said ankyrin repeat module(s) of SEQ ID NOs: 27 to 50.

In one particular embodiment, a designed ankyrin repeat domain with binding specificity for a target peptide-MHC complex according to the present invention comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 16 to 26, wherein G at position 1 and/or S at position 2 of SEQ ID NOs: 16 to 26 are optionally missing, and wherein A at the second last position of SEQ ID NOs: 16 to 26 is optionally substituted by L and/or A at the last position of SEQ ID NOs: 16 to 26 is optionally substituted by N. Thus, in one embodiment, said ankyrin repeat domain comprises an amino acid sequence with at least 80% amino acid sequence identity with any one of SEQ ID NOs: 16 to 26, wherein G at position 1 and/or S at position 2 of SEQ ID NOs: 16 to 26 are optionally missing, and wherein A at the second last position of SEQ ID NOs: 16 to 26 is optionally substituted by L and/or A at the last position of SEQ ID NOs: 16 to 26 is optionally substituted by N. Thus, in one embodiment, said ankyrin repeat domain comprises an amino acid sequence with at least 90% amino acid sequence identity with any one of SEQ ID NOs: 16 to 26. In another embodiment, said ankyrin repeat domain comprises an amino acid sequence with at least 93% amino acid sequence identity with any one of SEQ ID NOs: 16 to 26; and in a further embodiment, said ankyrin repeat domain comprises an amino acid sequence with at least 95% amino acid sequence identity with any one of SEQ ID NOs: 16 to 26. In one embodiment, said ankyrin repeat domain comprises an amino acid sequence with at least 98% amino acid sequence identity with any one of SEQ ID NOs: 16 to 26; and in one embodiment, said ankyrin repeat domain comprises the amino acid sequence of any one of SEQ ID NOs: 16 to 26. Thus, in one embodiment, said ankyrin repeat domain comprises an amino acid sequence selected from SEQ ID NOs: 16 to 26, wherein G at position 1 and/or S at position 2 of SEQ ID NOs: 16 to 26 are optionally missing, and wherein A at the second last position of SEQ ID NOs: 16 to 26 is optionally substituted by L and/or A at the last position of SEQ ID NOs: 16 to 26 is optionally substituted by N. In one preferred embodiment, said target peptide has the amino acid sequence of SEQ ID NO: 51.

In one particular embodiment, a designed ankyrin repeat domain with binding specificity for a target peptide-MHC complex according to the present invention comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 16 to 18 and 20 to 26, wherein G at position 1 and/or S at position 2 of SEQ ID NOs: 16 to 18 and 20 to 26 are optionally missing, and wherein A at the second last position of SEQ ID NOs: 16 to 18 and 20 to 26 is optionally substituted by L and/or A at the last position of SEQ ID NOs: 16 to 18 and 20 to 26 is optionally substituted by N.

In one particular embodiment, a designed ankyrin repeat domain with binding specificity for a target peptide-MHC complex according to the present invention comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 17, 21, 22, 23 and 26, wherein G at position 1 and/or S at position 2 of SEQ ID NOs: 17, 21, 22, 23 and 26 are optionally missing, and wherein A at the second last position of SEQ ID NOs: 17, 21, 22, 23 and 26 is optionally substituted by L and/or A at the last position of SEQ ID NOs: 17, 21, 22, 23 and 26 is optionally substituted by N. Thus, in one embodiment, said ankyrin repeat domain comprises an amino acid sequence with at least 80% amino acid sequence identity with any one of SEQ ID NOs: 17, 21, 22, 23 and 26, wherein G at position 1 and/or S at position 2 of SEQ ID NOs: 17, 21, 22, 23 and 26 are optionally missing, and wherein A at the second last position of SEQ ID NOs: 17, 21, 22, 23 and 26 is optionally substituted by L and/or A at the last position of SEQ ID NOs: 17, 21, 22, 23 and 26 is optionally substituted by N. Thus, in one embodiment, said ankyrin repeat domain comprises an amino acid sequence with at least 90% amino acid sequence identity with any one of SEQ ID NOs: 17, 21, 22, 23 and 26. In another embodiment, said ankyrin repeat domain comprises an amino acid sequence with at least 93% amino acid sequence identity with any one of SEQ ID NOs: 17, 21, 22, 23 and 26; and in a further embodiment, said ankyrin repeat domain comprises an amino acid sequence with at least 95% amino acid sequence identity with any one of SEQ ID NOs: 17, 21, 22, 23 and 26. In one embodiment, said ankyrin repeat domain comprises an amino acid sequence with at least 98% amino acid sequence identity with any one of SEQ ID NOs: 17, 21, 22, 23 and 26; and in one embodiment, said ankyrin repeat domain comprises the amino acid sequence of any one of SEQ ID NOs: 17, 21, 22, 23 and 26. Thus, in one embodiment, said ankyrin repeat domain comprises an amino acid sequence selected from SEQ ID NOs: 17, 21, 22, 23 and 26, wherein G at position 1 and/or S at position 2 of SEQ ID NOs: 17, 21, 22, 23 and 26 are optionally missing, and wherein A at the second last position of SEQ ID NOs: 17, 21, 22, 23 and 26 is optionally substituted by L and/or A at the last position of SEQ ID NOs: 17, 21, 22, 23 and 26 is optionally substituted by N. In one preferred embodiment, said target peptide has the amino acid sequence of SEQ ID NO: 51.

In one particular embodiment, a designed ankyrin repeat domain with binding specificity for a target peptide-MHC complex according to the present invention comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 21 and 23, wherein G at position 1 and/or S at position 2 of SEQ ID NOs: 21 and 23 are optionally missing, and wherein A at the second last position of SEQ ID NOs: 21 and 23 is optionally substituted by L and/or A at the last position of SEQ ID NOs: 21 and 23 is optionally substituted by N. Thus, in one embodiment, said ankyrin repeat domain comprises an amino acid sequence with at least 80% amino acid sequence identity with any one of SEQ ID NOs: 21 and 23, wherein G at position 1 and/or S at position 2 of SEQ ID NOs: 21 and 23 are optionally missing, and wherein A at the second last position of SEQ ID NOs: 21 and 23 is optionally substituted by L and/or A at the last position of SEQ ID NOs: 21 and 23 is optionally substituted by N. Thus, in one embodiment, said ankyrin repeat domain comprises an amino acid sequence with at least 90% amino acid sequence identity with any one of SEQ ID NOs: 21 and 23. In another embodiment, said ankyrin repeat domain comprises an amino acid sequence with at least 93% amino acid sequence identity with any one of SEQ ID NOs: 21 and 23; and in a further embodiment, said ankyrin repeat domain comprises an amino acid sequence with at least 95% amino acid sequence identity with any one of SEQ ID NOs: 21 and 23. In one embodiment, said ankyrin repeat domain comprises an amino acid sequence with at least 98% amino acid sequence identity with any one of SEQ ID NOs: 21 and 23; and in one embodiment, said ankyrin repeat domain comprises the amino acid sequence of any one of SEQ ID NOs: 21 and 23. Thus, in one embodiment, said ankyrin repeat domain comprises an amino acid sequence selected from SEQ ID NOs: 21 and 23, wherein G at position 1 and/or S at position 2 of SEQ ID NOs: 21 and 23 are optionally missing, and wherein A at the second last position of SEQ ID NOs: 21 and 23 is optionally substituted by L and/or A at the last position of SEQ ID NOs: 21 and 23 is optionally substituted by N. In one preferred embodiment, said target peptide has the amino acid sequence of SEQ ID NO: 51.

In one particular embodiment, a designed ankyrin repeat domain with binding specificity for a target peptide-MHC complex according to the present invention comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 17, wherein G at position 1 and/or S at position 2 of SEQ ID NO: 17 are optionally missing, and wherein A at the second last position of SEQ ID NO: 17 is optionally substituted by L and/or A at the last position of SEQ ID NO: 17 is optionally substituted by N. Thus, in one embodiment, said ankyrin repeat domain comprises an amino acid sequence with at least 80% amino acid sequence identity with SEQ ID NO: 17, wherein G at position 1 and/or S at position 2 of SEQ ID NO: 17 are optionally missing, and wherein A at the second last position of SEQ ID NO: 17 is optionally substituted by L and/or A at the last position of SEQ ID NO: 17 is optionally substituted by N. Thus, in one embodiment, said ankyrin repeat domain comprises an amino acid sequence with at least 90% amino acid sequence identity with SEQ ID NO: 17. In another embodiment, said ankyrin repeat domain comprises an amino acid sequence with at least 93% amino acid sequence identity with SEQ ID NO: 17; and in a further embodiment, said ankyrin repeat domain comprises an amino acid sequence with at least 95% amino acid sequence identity with SEQ ID NO: 17. In one embodiment, said ankyrin repeat domain comprises an amino acid sequence with at least 98% amino acid sequence identity with SEQ ID NO: 17; and in one embodiment, said ankyrin repeat domain comprises the amino acid sequence of SEQ ID NO: 17. Thus, in one embodiment, said ankyrin repeat domain comprises the amino acid sequence of SEQ ID NO: 17, wherein G at position 1 and/or S at position 2 of SEQ ID NO: 17 are optionally missing, and wherein A at the second last position of SEQ ID NO: 17 is optionally substituted by L and/or A at the last position of SEQ ID NO: 17 is optionally substituted by N. In one preferred embodiment, said target peptide has the amino acid sequence of SEQ ID NO: 51.

In one particular embodiment, a designed ankyrin repeat domain with binding specificity for a target peptide-MHC complex according to the present invention comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 21, wherein G at position 1 and/or S at position 2 of SEQ ID NO: 21 are optionally missing, and wherein A at the second last position of SEQ ID NO: 21 is optionally substituted by L and/or A at the last position of SEQ ID NO: 21 is optionally substituted by N. Thus, in one embodiment, said ankyrin repeat domain comprises an amino acid sequence with at least 80% amino acid sequence identity with SEQ ID NO: 21, wherein G at position 1 and/or S at position 2 of SEQ ID NO: 21 are optionally missing, and wherein A at the second last position of SEQ ID NO: 21 is optionally substituted by L and/or A at the last position of SEQ ID NO: 21 is optionally substituted by N. Thus, in one embodiment, said ankyrin repeat domain comprises an amino acid sequence with at least 90% amino acid sequence identity with SEQ ID NO: 21. In another embodiment, said ankyrin repeat domain comprises an amino acid sequence with at least 93% amino acid sequence identity with SEQ ID NO: 21; and in a further embodiment, said ankyrin repeat domain comprises an amino acid sequence with at least 95% amino acid sequence identity with SEQ ID NO: 21. In one embodiment, said ankyrin repeat domain comprises an amino acid sequence with at least 98% amino acid sequence identity with SEQ ID NO: 21; and in one embodiment, said ankyrin repeat domain comprises the amino acid sequence of SEQ ID NO: 21. Thus, in one embodiment, said ankyrin repeat domain comprises the amino acid sequence of SEQ ID NO: 21, wherein G at position 1 and/or S at position 2 of SEQ ID NO: 21 are optionally missing, and wherein A at the second last position of SEQ ID NO: 21 is optionally substituted by L and/or A at the last position of SEQ ID NO: 21 is optionally substituted by N. In one preferred embodiment, said target peptide has the amino acid sequence of SEQ ID NO: 51.

In one particular embodiment, a designed ankyrin repeat domain with binding specificity for a target peptide-MHC complex according to the present invention comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 22, wherein G at position 1 and/or S at position 2 of SEQ ID NO: 22 are optionally missing, and wherein A at the second last position of SEQ ID NO: 22 is optionally substituted by L and/or A at the last position of SEQ ID NO: 22 is optionally substituted by N. Thus, in one embodiment, said ankyrin repeat domain comprises an amino acid sequence with at least 80% amino acid sequence identity with SEQ ID NO: 22, wherein G at position 1 and/or S at position 2 of SEQ ID NO: 22 are optionally missing, and wherein A at the second last position of SEQ ID NO: 22 is optionally substituted by L and/or A at the last position of SEQ ID NO: 27 is optionally substituted by N. Thus, in one embodiment, said ankyrin repeat domain comprises an amino acid sequence with at least 90% amino acid sequence identity with SEQ ID NO: 22. In another embodiment, said ankyrin repeat domain comprises an amino acid sequence with at least 93% amino acid sequence identity with SEQ ID NO: 22; and in a further embodiment, said ankyrin repeat domain comprises an amino acid sequence with at least 95% amino acid sequence identity with SEQ ID NO: 22. In one embodiment, said ankyrin repeat domain comprises an amino acid sequence with at least 98% amino acid sequence identity with SEQ ID NO: 22; and in one embodiment, said ankyrin repeat domain comprises the amino acid sequence of SEQ ID NO: 22. Thus, in one embodiment, said ankyrin repeat domain comprises the amino acid sequence of SEQ ID NO: 22, wherein G at position 1 and/or S at position 2 of SEQ ID NO: 22 are optionally missing, and wherein A at the second last position of SEQ ID NO: 22 is optionally substituted by L and/or A at the last position of SEQ ID NO: 22 is optionally substituted by N. In one preferred embodiment, said target peptide has the amino acid sequence of SEQ ID NO: 51.

In one particular embodiment, a designed ankyrin repeat domain with binding specificity for a target peptide-MHC complex according to the present invention comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 23, wherein G at position 1 and/or S at position 2 of SEQ ID NO: 23 are optionally missing, and wherein A at the second last position of SEQ ID NO: 23 is optionally substituted by L and/or A at the last position of SEQ ID NO: 23 is optionally substituted by N. Thus, in one embodiment, said ankyrin repeat domain comprises an amino acid sequence with at least 80% amino acid sequence identity with SEQ ID NO: 23, wherein G at position 1 and/or S at position 2 of SEQ ID NO: 23 are optionally missing, and wherein A at the second last position of SEQ ID NO: 23 is optionally substituted by L and/or A at the last position of SEQ ID NO: 23 is optionally substituted by N. Thus, in one embodiment, said ankyrin repeat domain comprises an amino acid sequence with at least 90% amino acid sequence identity with SEQ ID NO: 23. In another embodiment, said ankyrin repeat domain comprises an amino acid sequence with at least 93% amino acid sequence identity with SEQ ID NO: 23; and in a further embodiment, said ankyrin repeat domain comprises an amino acid sequence with at least 95% amino acid sequence identity with SEQ ID NO: 23. In one embodiment, said ankyrin repeat domain comprises an amino acid sequence with at least 98% amino acid sequence identity with SEQ ID NO: 23; and in one embodiment, said ankyrin repeat domain comprises the amino acid sequence of SEQ ID NO: 23. Thus, in one embodiment, said ankyrin repeat domain comprises the amino acid sequence of SEQ ID NO: 23, wherein G at position 1 and/or S at position 2 of SEQ ID NO: 23 are optionally missing, and wherein A at the second last position of SEQ ID NO: 23 is optionally substituted by L and/or A at the last position of SEQ ID NO: 23 is optionally substituted by N. In one preferred embodiment, said target peptide has the amino acid sequence of SEQ ID NO: 51.

In one particular embodiment, a designed ankyrin repeat domain with binding specificity for a target peptide-MHC complex according to the present invention comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 26, wherein G at position 1 and/or S at position 2 of SEQ ID NO: 26 are optionally missing, and wherein A at the second last position of SEQ ID NO: 26 is optionally substituted by L and/or A at the last position of SEQ ID NO: 26 is optionally substituted by N. Thus, in one embodiment, said ankyrin repeat domain comprises an amino acid sequence with at least 80% amino acid sequence identity with SEQ ID NO: 26, wherein G at position 1 and/or S at position 2 of SEQ ID NO: 26 are optionally missing, and wherein A at the second last position of SEQ ID NO: 26 is optionally substituted by L and/or A at the last position of SEQ ID NO: 26 is optionally substituted by N. Thus, in one embodiment, said ankyrin repeat domain comprises an amino acid sequence with at least 90% amino acid sequence identity with SEQ ID NO: 26. In another embodiment, said ankyrin repeat domain comprises an amino acid sequence with at least 93% amino acid sequence identity with SEQ ID NO: 26; and in a further embodiment, said ankyrin repeat domain comprises an amino acid sequence with at least 95% amino acid sequence identity with SEQ ID NO: 26. In one embodiment, said ankyrin repeat domain comprises an amino acid sequence with at least 98% amino acid sequence identity with SEQ ID NO: 26; and in one embodiment, said ankyrin repeat domain comprises the amino acid sequence of SEQ ID NO: 26. Thus, in one embodiment, said ankyrin repeat domain comprises the amino acid sequence of SEQ ID NO: 26, wherein G at position 1 and/or S at position 2 of SEQ ID NO: 26 are optionally missing, and wherein A at the second last position of SEQ ID NO: 26 is optionally substituted by L and/or A at the last position of SEQ ID NO: 26 is optionally substituted by N. In one preferred embodiment, said target peptide has the amino acid sequence of SEQ ID NO: 51.

In one embodiment, a designed ankyrin repeat domain with binding specificity for a target peptide-MHC complex according to the present invention binds said target peptide-MHC complex in PBS with a dissociation constant (K_{D}) below 10⁻⁷M, or below 5×10⁻⁸M, or below 3×10⁻⁸M, or below 2×10⁻⁸M, or below 10⁻⁸M, or below 5×10⁻⁹M, or below 3×10⁻⁹M, or below 2×10⁻⁹M, or below 10⁻⁹M, or below 5×10⁻¹⁰M, or below 3×10⁻¹⁰M, or below 2×10⁻¹⁰M, or below 10⁻¹⁰M. Thus, in one embodiment, said ankyrin repeat domain binds said target peptide-MHC complex in PBS with a dissociation constant (K_{D}) below 10⁻⁷M. In one embodiment, said ankyrin repeat domain binds said target peptide-MHC complex in PBS with a dissociation constant (K_{D}) below 5×10⁻⁸M. In one embodiment, said ankyrin repeat domain binds said target peptide-MHC complex in PBS with a dissociation constant (K_{D}) below 3×10⁻⁸M. In one embodiment, said ankyrin repeat domain binds said target peptide-MHC complex in PBS with a dissociation constant (K_{D}) below 2×10⁻⁸M. In one embodiment, said ankyrin repeat domain binds said target peptide-MHC complex in PBS with a dissociation constant (K_{D}) below 10⁻⁸M. In one embodiment, said ankyrin repeat domain binds said target peptide-MHC complex in PBS with a dissociation constant (K_{D}) below 5×10⁻⁹M. In one embodiment, said ankyrin repeat domain binds said target peptide-MHC complex in PBS with a dissociation constant (K_{D}) below 3×10⁻⁹M. In one embodiment, said ankyrin repeat domain binds said target peptide-MHC complex in PBS with a dissociation constant (K_{D}) below 2×10⁻⁹M. In one embodiment, said ankyrin repeat domain binds said target peptide-MHC complex in PBS with a dissociation constant (K_{D}) below 10⁻⁹M. In one embodiment, said ankyrin repeat domain binds said target peptide-MHC complex in PBS with a dissociation constant (K_{D}) below 5×10⁻¹⁰M. In one embodiment, said ankyrin repeat domain binds said target peptide-MHC complex in PBS with a dissociation constant (K_{D}) below 3×10⁻¹⁰M. In one embodiment, said ankyrin repeat domain binds said target peptide-MHC complex in PBS with a dissociation constant (K_{D}) below 2×10⁻¹⁰M. In one embodiment, said ankyrin repeat domain binds said target peptide-MHC complex in PBS with a dissociation constant (K_{D}) below 10⁻¹⁰M.

In one embodiment, a designed ankyrin repeat domain with binding specificity for a target peptide-MHC complex according to the present invention binds said target peptide-MHC complex in PBS with a dissociation constant (K_{D}) below 10⁻⁷M, and comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 16 to 26, wherein G at position 1 and/or S at position 2 of SEQ ID NOs: 16 to 26 are optionally missing, and wherein A at the second last position of SEQ ID NOs: 16 to 26 is optionally substituted by L and/or A at the last position of SEQ ID NOs: 16 to 26 is optionally substituted by N. Thus, in one embodiment, said ankyrin repeat domain comprises an amino acid sequence with at least 90% amino acid sequence identity with any one of SEQ ID NOs: 16 to 26. In another embodiment, said ankyrin repeat domain comprises an amino acid sequence with at least 93% amino acid sequence identity with any one of SEQ ID NOs: 16 to 26; and in a further embodiment, said ankyrin repeat domain comprises an amino acid sequence with at least 95% amino acid sequence identity with any one of SEQ ID NOs: 16 to 26. In one embodiment, said ankyrin repeat domain comprises an amino acid sequence with at least 98% amino acid sequence identity with any one of SEQ ID NOs: 16 to 26; and in one embodiment, said ankyrin repeat domain comprises the amino acid sequence of any one of SEQ ID NOs: 16 to 26. Thus, in one embodiment, a designed ankyrin repeat domain with binding specificity for a target peptide-MHC complex according to the present invention binds said target peptide-MHC complex in PBS with a dissociation constant (K_{D}) below 10⁻⁷M, and comprises the amino acid sequence of any one of SEQ ID NOs: 16 to 26, wherein G at position 1 and/or S at position 2 of any one of SEQ ID NOs: 16 to 26 are optionally missing, and wherein A at the second last position of SEQ ID NOs: 16 to 26 is optionally substituted by L and/or A at the last position of SEQ ID NOs: 16 to 26 is optionally substituted by N. In one preferred embodiment, said target peptide has the amino acid sequence of SEQ ID NO: 51.

In one embodiment, a designed ankyrin repeat domain with binding specificity for a target peptide-MHC complex according to the present invention binds said target peptide-MHC complex in PBS with a dissociation constant (K_{D}) below 10⁻⁷M, and comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 16 to 18 and 20 to 26, wherein G at position 1 and/or S at position 2 of SEQ ID NOs: 16 to 18 and 20 to 26 are optionally missing, and wherein A at the second last position of SEQ ID NOs: 16 to 18 and 20 to 26 is optionally substituted by L and/or A at the last position of SEQ ID NOs: 16 to 18 and 20 to 26 is optionally substituted by N.

In one embodiment, a designed ankyrin repeat domain with binding specificity for a target peptide-MHC complex according to the present invention binds said target peptide-MHC complex in PBS with a dissociation constant (K_{D}) below 5×10⁻⁸M, and comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 16 to 26, wherein G at position 1 and/or S at position 2 of SEQ ID NOs: 16 to 26 are optionally missing, and wherein A at the second last position of SEQ ID NOs: 16 to 26 is optionally substituted by L and/or A at the last position of SEQ ID NOs: 16 to 26 is optionally substituted by N. Thus, in one embodiment, said ankyrin repeat domain comprises an amino acid sequence with at least 90% amino acid sequence identity with any one of SEQ ID NOs: 16 to 26. In another embodiment, said ankyrin repeat domain comprises an amino acid sequence with at least 93% amino acid sequence identity with any one of SEQ ID NOs: 16 to 26; and in a further embodiment, said ankyrin repeat domain comprises an amino acid sequence with at least 95% amino acid sequence identity with any one of SEQ ID NOs: 16 to 26. In one embodiment, said ankyrin repeat domain comprises an amino acid sequence with at least 98% amino acid sequence identity with any one of SEQ ID NOs: 16 to 26; and in one embodiment, said ankyrin repeat domain comprises the amino acid sequence of any one of SEQ ID NOs: 16 to 26. Thus, in one embodiment, a designed ankyrin repeat domain with binding specificity for a target peptide-MHC complex according to the present invention binds said target peptide-MHC complex in PBS with a dissociation constant (K_{D}) below 5×10⁻⁸M, and comprises the amino acid sequence of any one of SEQ ID NOs: 16 to 26, wherein G at position 1 and/or S at position 2 of any one of SEQ ID NOs: 16 to 26 are optionally missing, and wherein A at the second last position of SEQ ID NOs: 16 to 26 is optionally substituted by L and/or A at the last position of SEQ ID NOs: 16 to 26 is optionally substituted by N. In one preferred embodiment, said target peptide has the amino acid sequence of SEQ ID NO: 51.

In one embodiment, a designed ankyrin repeat domain with binding specificity for a target peptide-MHC complex according to the present invention binds said first target peptide-MHC complex in PBS with a dissociation constant (K_{D}) below 2×10⁻⁸M, and comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 16 to 26, wherein G at position 1 and/or S at position 2 of SEQ ID NOs: 16 to 26 are optionally missing, and wherein A at the second last position of SEQ ID NOs: 16 to 26 is optionally substituted by L and/or A at the last position of SEQ ID NOs: 16 to 26 is optionally substituted by N. Thus, in one embodiment, said ankyrin repeat domain comprises an amino acid sequence with at least 90% amino acid sequence identity with any one of SEQ ID NOs: 16 to 26. In another embodiment, said ankyrin repeat domain comprises an amino acid sequence with at least 93% amino acid sequence identity with any one of SEQ ID NOs: 16 to 26; and in a further embodiment, said ankyrin repeat domain comprises an amino acid sequence with at least 95% amino acid sequence identity with any one of SEQ ID NOs: 16 to 26. In one embodiment, said ankyrin repeat domain comprises an amino acid sequence with at least 98% amino acid sequence identity with any one of SEQ ID NOs: 16 to 26; and in one embodiment, said ankyrin repeat domain comprises the amino acid sequence of any one of SEQ ID NOs: 16 to 26. Thus, in one embodiment, a designed ankyrin repeat domain with binding specificity for a target peptide-MHC complex according to the present invention binds said target peptide-MHC complex in PBS with a dissociation constant (K_{D}) below 2×10⁻⁸M, and comprises the amino acid sequence of any one of SEQ ID NOs: 16 to 26, wherein G at position 1 and/or S at position 2 of any one of SEQ ID NOs: 16 to 26 are optionally missing, and wherein A at the second last position of SEQ ID NOs: 16 to 26 is optionally substituted by L and/or A at the last position of SEQ ID NOs: 16 to 26 is optionally substituted by N. In one preferred embodiment, said target peptide has the amino acid sequence of SEQ ID NO: 51.

In one embodiment, a designed ankyrin repeat domain with binding specificity for a target peptide-MHC complex according to the present invention binds said target peptide-MHC complex in PBS with a dissociation constant (K_{D}) below 10⁻⁷M, or below 5×10⁻⁸M, or below 3×10⁻⁸M, or below 2×10⁻⁸M, or below 10⁻⁸M, or below 5×10⁻⁹M, or below 3×10⁻⁹M, or below 2×10⁻⁹M, or below 10⁻⁹M, 5×10⁻¹⁰M, and comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 17, wherein G at position 1 and/or S at position 2 of SEQ ID NO: 17 are optionally missing, and wherein A at the second last position of SEQ ID NO: 17 is optionally substituted by L and/or A at the last position of SEQ ID NO: 17 is optionally substituted by N. Thus, in one embodiment, said ankyrin repeat domain comprises an amino acid sequence with at least 90% amino acid sequence identity with SEQ ID NO: 17. In another embodiment, said ankyrin repeat domain comprises an amino acid sequence with at least 93% amino acid sequence identity with SEQ ID NO: 17; and in a further embodiment, said ankyrin repeat domain comprises an amino acid sequence with at least 95% amino acid sequence identity with SEQ ID NO: 17. In one embodiment, said ankyrin repeat domain comprises an amino acid sequence with at least 98% amino acid sequence identity with SEQ ID NO: 17; and in one embodiment, said ankyrin repeat domain comprises the amino acid sequence of SEQ ID NO: 17. Thus, in one embodiment, a designed ankyrin repeat domain with binding specificity for a target peptide-MHC complex according to the present invention binds said target peptide-MHC complex in PBS with a dissociation constant (K_{D}) below 10⁻⁷M, or below 5×10⁻⁸M, or below 3×10⁻⁸M, or below 2×10⁻⁸M, or below 10⁻⁸M, or below 5×10⁻⁹M, or below 3×10⁻⁹M, or below 2×10⁻⁹M, or below 10⁻⁹M, 5×10⁻¹⁰M, and comprises the amino acid sequence of SEQ ID NO: 17, wherein G at position 1 and/or S at position 2 of SEQ ID NO: 17 are optionally missing, and wherein A at the second last position of SEQ ID NO: 17 is optionally substituted by L and/or A at the last position of SEQ ID NO: 17 is optionally substituted by N. In one preferred embodiment, said target peptide has the amino acid sequence of SEQ ID NO: 51.

In one embodiment, a designed ankyrin repeat domain with binding specificity for a target peptide-MHC complex according to the present invention binds said target peptide-MHC complex in PBS with a dissociation constant (K_{D}) below 10⁻⁷M, or below 5×10⁻⁸M, or below 3×10⁻⁸M, or below 2×10⁻⁸M, or below 10⁻⁸M, and comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 21, wherein G at position 1 and/or S at position 2 of SEQ ID NO: 21 are optionally missing, and wherein A at the second last position of SEQ ID NO: 21 is optionally substituted by L and/or A at the last position of SEQ ID NO: 21 is optionally substituted by N. Thus, in one embodiment, said ankyrin repeat domain comprises an amino acid sequence with at least 90% amino acid sequence identity with SEQ ID NO: 21. In another embodiment, said ankyrin repeat domain comprises an amino acid sequence with at least 93% amino acid sequence identity with SEQ ID NO: 21; and in a further embodiment, said ankyrin repeat domain comprises an amino acid sequence with at least 95% amino acid sequence identity with SEQ ID NO: 21. In one embodiment, said ankyrin repeat domain comprises an amino acid sequence with at least 98% amino acid sequence identity with SEQ ID NO: 21; and in one embodiment, said ankyrin repeat domain comprises the amino acid sequence of SEQ ID NO: 21. Thus, in one embodiment, a designed ankyrin repeat domain with binding specificity for a target peptide-MHC complex according to the present invention binds said target peptide-MHC complex in PBS with a dissociation constant (K_{D}) below 10⁻⁷M, or below 5×10⁻⁸M, or below 3×10⁻⁸M, or below 2×10⁻⁸M, or below 10⁻⁸M, and comprises the amino acid sequence of SEQ ID NO: 21, wherein G at position 1 and/or S at position 2 of SEQ ID NO: 21 are optionally missing, and wherein A at the second last position of SEQ ID NO: 21 is optionally substituted by L and/or A at the last position of SEQ ID NO: 21 is optionally substituted by N. In one preferred embodiment, said target peptide has the amino acid sequence of SEQ ID NO: 51.

In one embodiment, a designed ankyrin repeat domain with binding specificity for a target peptide-MHC complex according to the present invention binds said first target peptide-MHC complex in PBS with a dissociation constant (K_{D}) below 10⁻⁷M, or below 5×10⁻⁸M, or below 3×10⁻⁸M, or below 2×10⁻⁸M, and comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 22, wherein G at position 1 and/or S at position 2 of SEQ ID NO: 22 are optionally missing, and wherein A at the second last position of SEQ ID NO: 22 is optionally substituted by L and/or A at the last position of SEQ ID NO: 22 is optionally substituted by N. Thus, in one embodiment, said ankyrin repeat domain comprises an amino acid sequence with at least 90% amino acid sequence identity with SEQ ID NO: 22. In another embodiment, said ankyrin repeat domain comprises an amino acid sequence with at least 93% amino acid sequence identity with SEQ ID NO: 22; and in a further embodiment, said ankyrin repeat domain comprises an amino acid sequence with at least 95% amino acid sequence identity with SEQ ID NO: 22. In one embodiment, said ankyrin repeat domain comprises an amino acid sequence with at least 98% amino acid sequence identity with SEQ ID NO: 22; and in one embodiment, said ankyrin repeat domain comprises the amino acid sequence of SEQ ID NO: 22. Thus, in one embodiment, a designed ankyrin repeat domain with binding specificity for a target peptide-MHC complex according to the present invention binds said target peptide-MHC complex in PBS with a dissociation constant (K_{D}) below 10⁻⁷M, or below 5×10⁻⁸M, or below 3×10⁻⁸M, or below 2×10⁻⁸M, and comprises the amino acid sequence of SEQ ID NO: 22, wherein G at position 1 and/or S at position 2 of SEQ ID NO: 22 are optionally missing, and wherein A at the second last position of SEQ ID NO: 22 is optionally substituted by L and/or A at the last position of SEQ ID NO: 22 is optionally substituted by N. In one preferred embodiment, said target peptide has the amino acid sequence of SEQ ID NO: 51.

In one embodiment, a designed ankyrin repeat domain with binding specificity for a target peptide-MHC complex according to the present invention binds said target peptide-MHC complex in PBS with a dissociation constant (K_{D}) below 10⁻⁷M, or below 5×10⁻⁸M, or below 2×10⁻⁸M, or below 10⁻⁸M, and comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 23, wherein G at position 1 and/or S at position 2 of SEQ ID NO: 23 are optionally missing, and wherein A at the second last position of SEQ ID NO: 23 is optionally substituted by L and/or A at the last position of SEQ ID NO: 23 is optionally substituted by N. Thus, in one embodiment, said ankyrin repeat domain comprises an amino acid sequence with at least 90% amino acid sequence identity with SEQ ID NO: 23. In another embodiment, said ankyrin repeat domain comprises an amino acid sequence with at least 93% amino acid sequence identity with SEQ ID NO: 23; and in a further embodiment, said ankyrin repeat domain comprises an amino acid sequence with at least 95% amino acid sequence identity with SEQ ID NO: 23. In one embodiment, said ankyrin repeat domain comprises an amino acid sequence with at least 98% amino acid sequence identity with SEQ ID NO: 23; and in one embodiment, said ankyrin repeat domain comprises the amino acid sequence of SEQ ID NO: 23. Thus, in one embodiment, a designed ankyrin repeat domain with binding specificity for a target peptide-MHC complex according to the present invention binds said target peptide-MHC complex in PBS with a dissociation constant (K_{D}) below 10⁻⁷M, or below 5×10⁻⁸M, or below 2×10⁻⁸M, or below 10⁻⁸M, and comprises the amino acid sequence of SEQ ID NO: 23, wherein G at position 1 and/or S at position 2 of SEQ ID NO: 23 are optionally missing, and wherein A at the second last position of SEQ ID NO: 23 is optionally substituted by L and/or A at the last position of SEQ ID NO: 23 is optionally substituted by N. In one preferred embodiment, said target peptide has the amino acid sequence of SEQ ID NO: 51.

In one embodiment, a designed ankyrin repeat domain with binding specificity for a target peptide-MHC complex according to the present invention binds said target peptide-MHC complex in PBS with a dissociation constant (K_{D}) below 10⁻⁷M, or below 5×10⁻⁸M, or below 3×10⁻⁸M, or below 2×10⁻⁸M, or below 10⁻⁸M, and comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 26 wherein G at position 1 and/or S at position 2 of SEQ ID NO: 26 are optionally missing, and wherein A at the second last position of SEQ ID NO: 26 is optionally substituted by L and/or A at the last position of SEQ ID NO: 26 is optionally substituted by N. Thus, in one embodiment, said ankyrin repeat domain comprises an amino acid sequence with at least 90% amino acid sequence identity with SEQ ID NO: 26. In another embodiment, said ankyrin repeat domain comprises an amino acid sequence with at least 93% amino acid sequence identity with SEQ ID NO: 26; and in a further embodiment, said ankyrin repeat domain comprises an amino acid sequence with at least 95% amino acid sequence identity with SEQ ID NO: 26. In one embodiment, said ankyrin repeat domain comprises an amino acid sequence with at least 98% amino acid sequence identity with SEQ ID NO: 26; and in one embodiment, said ankyrin repeat domain comprises the amino acid sequence of SEQ ID NO: 26. Thus, in one embodiment, a designed ankyrin repeat domain with binding specificity for a target peptide-MHC complex according to the present invention binds said first target peptide-MHC complex in PBS with a dissociation constant (K_{D}) below 10⁻⁷M, or below 5×10⁻⁸M, or below 3×10⁻⁸M, or below 2×10⁻⁸M, or below 10⁻⁸M, and comprises the amino acid sequence of SEQ ID NO: 26, wherein G at position 1 and/or S at position 2 of SEQ ID NO: 26 are optionally missing, and wherein A at the second last position of SEQ ID NO: 26 is optionally substituted by L and/or A at the last position of SEQ ID NO: 26 is optionally substituted by N. In one preferred embodiment, said target peptide has the amino acid sequence of SEQ ID NO: 51.

In the context of the present invention, a typical and preferred determination of dissociation constants (K_{D}) of the recombinant binding proteins or designed repeat domains of the invention with binding specificity for a target peptide-MHC complex by Surface Plasmon Resonance (SPR) analysis is described in Example 2. Thus, in one embodiment said binding specificity for a target peptide-MHC complex of the recombinant binding proteins or designed repeat domains of the invention is determined in PBS by Surface Plasmon Resonance (SPR). In one embodiment said binding specificity for a target peptide-MHC complex of the recombinant binding proteins or designed repeat domains of the invention is determined in PBS by Surface Plasmon Resonance (SPR) as described in Example 2.

In one aspect of the invention, the recombinant binding protein of the invention further comprises a second designed repeat domain with binding specificity for a second target peptide-MHC complex.

In a preferred embodiment, said second target peptide is selected from the group consisting of (i) a peptide derived from a protein expressed in a tumor cell, (ii) a peptide derived from a protein of an infectious agent, such as a bacterial infectious agent or a viral infectious agent, preferably a viral infectious agent, and (iii) a peptide derived from a protein associated with an autoimmune disorder.

In one embodiment, said second target peptide is derived from a protein of a viral infectious agent, preferably a virus-specific protein. In one embodiment, said second target peptide is derived from a virus-specific protein.

In one embodiment, said second repeat domain with binding specificity for a second target peptide-MHC complex binds to said second target peptide-MHC complex in PBS with a dissociation constant (K_{D}) below 10⁻⁷M, or below 5×10⁻⁸M, or below 3×10⁻⁸M, or below 2×10⁻⁸M, or below 10⁻⁸M, or below 5×10⁻⁹M, or below 3×10⁻⁹M, or below 2×10⁻⁹M, or below 10⁻⁹M, or below 5×10⁻¹⁰M, or below 3×10⁻¹⁰M, or below 2×10⁻¹⁰M, or below 10⁻¹⁰M. In one embodiment, said second repeat domain with binding specificity for a second target peptide-MHC complex binds to said second target peptide-MHC complex in PBS with a dissociation constant (K_{D}) below 10⁻⁷M.

In one embodiment, said binding of said second repeat domain to said second target peptide-MHC complex comprises interaction of said second repeat domain with at least one, at least two, at least three, at least four, at least five, at least six, or at least seven amino acid residues of said second target peptide. Thus, in one embodiment, said binding of said second repeat domain to said second target peptide-MHC complex comprises interaction of said second repeat domain with at least one amino acid residue of said second target peptide. In one embodiment, said binding of said second repeat domain to said second target peptide-MHC complex comprises interaction of said second repeat domain with at least two amino acid residues of said second target peptide. In one embodiment, said binding of said second repeat domain to said second target peptide-MHC complex comprises interaction of said second repeat domain with at least three amino acid residues of said second target peptide. In one embodiment, said binding of said second repeat domain to said second target peptide-MHC complex comprises interaction of said second repeat domain with at least four amino acid residues of said second target peptide. In one embodiment, said binding of said second repeat domain to said second target peptide-MHC complex comprises interaction of said second repeat domain with at least five amino acid residues of said second target peptide. In one embodiment, said binding of said second repeat domain to said second target peptide-MHC complex comprises interaction of said second repeat domain with at least six amino acid residues of said second target peptide. In one embodiment, said binding of said second repeat domain to said second target peptide-MHC complex comprises interaction of said second repeat domain with at least seven amino acid residues of said second target peptide. In a further or alternative embodiment, said binding of said second repeat domain to said second target peptide-MHC complex comprises interaction of said second repeat domain with at least one amino acid residue of said second MHC.

In one embodiment, said second MHC is MHC class I. In one embodiment, said second MHC is HLA-A*02. In one embodiment, said HLA-A*02 is HLA-A*0201. In one embodiment, said HLA-A*0201 has the amino acid sequence of SEQ ID NO: 69. Alternatively, in another embodiment, said second MHC is not HLA-A*02.

In one embodiment, said second target peptide is derived from the same protein as said first target peptide. In one embodiment, said second target peptide has the same amino acid sequence as said first target peptide. In a further embodiment, said second repeat domain has the same amino acid sequence as said first repeat domain. Alternatively, in another further embodiment, said second repeat domain has a different amino acid sequence as compared to said first repeat domain. In one embodiment, said second target peptide has a different amino acid sequence as compared to said first target peptide.

In another embodiment, said second target peptide is derived from a protein that is different from the protein, from which said first target peptide is derived.

In a preferred embodiment, said second designed repeat domain is a designed ankyrin repeat domain. In a particularly preferred embodiment, said second designed ankyrin repeat domain is a designed ankyrin repeat domain with binding specificity for a target peptide-MHC complex as described more specifically in any of the aspects and embodiments herein.

In one aspect of the invention, the recombinant binding protein of the invention further comprises a third designed repeat domain with binding specificity for a third target peptide-MHC complex. In a preferred embodiment, said third designed repeat domain is a designed ankyrin repeat domain. In a particularly preferred embodiment, said third designed ankyrin repeat domain is a designed ankyrin repeat domain with binding specificity for a target peptide-MHC complex as described more specifically in any of the aspects and embodiments herein.

In the context of the present invention, when the recombinant binding protein of the invention comprises a first and a second designed repeat domain, and when said first and said second repeat domains have binding specificity for the same target peptide-MHC complex, i.e. the second target peptide has the same amino acid sequence as the first target peptide, and when said first and said second repeat domains are identical in sequence, said recombinant binding protein is bivalent. In the context of the present invention, when the recombinant binding protein of the invention comprises a first and a second designed repeat domain, and when said first and said second repeat domains have binding specificity for the same target peptide-MHC complex, i.e. the second target peptide has the same amino acid sequence as the first target peptide, and when said first and said second repeat domains are different in sequence, said recombinant binding protein is biparatopic. In the context of the present invention, when the recombinant binding protein of the invention comprises a first and a second designed repeat domain, and when said first and said second repeat domains have binding specificity for different target peptide-MHC complexes, i.e. the second target peptide has a different amino acid sequence as compared to the first target peptide, said recombinant binding protein is bispecific. The different target peptides may be derived from the same protein or from different proteins. In the context of the present invention, the same definitions apply analogously to a recombinant binding protein of the invention which comprises a first and a second and a third designed repeat domain, wherein each of the first, second and third designed repeat domains has binding specificity for a target peptide-MHC complex.

In one embodiment, the recombinant binding protein of the invention is monovalent, bivalent, trivalent, multivalent, monoparatopic, biparatopic, triparatopic, multiparatopic, monospecific, bispecific, trispecific, or multispecific. In one particular embodiment, the recombinant binding protein of the invention is monovalent. In one particular embodiment, the recombinant binding protein of the invention is bivalent. In one particular embodiment, the recombinant binding protein of the invention is trivalent. In one particular embodiment, the recombinant binding protein of the invention is multivalent. In one particular embodiment, the recombinant binding protein of the invention is monoparatopic. In one particular embodiment, the recombinant binding protein of the invention is biparatopic. In one particular embodiment, the recombinant binding protein of the invention is triparatopic, In one particular embodiment, the recombinant binding protein of the invention is multiparatopic. In one particular embodiment, the recombinant binding protein of the invention is monospecific. In one particular embodiment, the recombinant binding protein of the invention is bispecific. In one particular embodiment, the recombinant binding protein of the invention is trispecific. In one particular embodiment, the recombinant binding protein of the invention is multispecific. It should be understood that the above embodiments are combinable with each other. As an example, a recombinant binding protein of the invention comprising two identical repeat domains with specificity for a first pMHC complex and further comprising a third repeat domain with specificity for a second, different pMHC complex would be bivalent and bispecific at the same time. Accordingly, in one particular embodiment, the recombinant binding protein of the invention is any combination of monovalent, bivalent, trivalent or multivalent, and monoparatopic, biparatopic, triparatopic or multiparatopic, and/or monospecific, bispecific, trispecific, or multispecific.

In one embodiment, when the recombinant binding protein of the invention comprises two or more ankyrin repeat domains, for example when the recombinant binding protein of the invention comprises two or three ankyrin repeat domains, said two or more ankyrin repeat domains, for example said two or three ankyrin repeat domains, may be linked with a peptide linker. In one embodiment, said peptide linker is a proline-threonine rich peptide linker. In one embodiment, said peptide linker is the proline-threonine rich peptide linker of SEQ ID NO: 1 or 2. In one embodiment, said two or more ankyrin repeat domains, for example said two or three ankyrin repeat domains, are linked with the proline-threonine rich peptide linker of SEQ ID NO: 1 or 2. In another embodiment, said peptide linker is a glycine-serine rich peptide linker. In one embodiment, said peptide linker is the glycine-serine rich peptide linker of SEQ ID NO: 3. In one embodiment, said two or more ankyrin repeat domains, for example said two or three ankyrin repeat domains, are linked with the glycine-serine rich peptide linker of SEQ ID NO: 3. In one embodiment, when the recombinant binding protein of the invention comprises three or more ankyrin repeat domains, said three or more ankyrin repeat domains may be linked with different peptide linkers, for example, proline-threonine rich peptide linkers and serine-glycine rich peptides linkers, such as, for example, the peptide linkers of SEQ ID NOs: 1 to 3.

In one embodiment, the recombinant binding protein of the invention comprises two or three ankyrin repeat domains, wherein each of said two or three ankyrin repeat domains independently comprises an ankyrin repeat module as described more specifically in any of the aspects and embodiments herein.

In one embodiment, the recombinant binding protein comprises two ankyrin repeat domains with binding specificity for a target peptide-MHC complex, wherein each of said two ankyrin repeat domains independently comprises an ankyrin repeat module as described more specifically in any of the aspects and embodiments herein.

In one embodiment, the recombinant binding protein of the invention comprises two ankyrin repeat domains with binding specificity for a target peptide-MHC complex, wherein each of said two ankyrin repeat domains comprises a first ankyrin repeat module and a second ankyrin repeat module as described more specifically in any of the aspects and embodiments herein.

In one embodiment, the recombinant binding protein of the invention comprises two ankyrin repeat domains with binding specificity for a target peptide-MHC complex, wherein each of said two ankyrin repeat domains comprises a first ankyrin repeat module, a second ankyrin repeat module and a third ankyrin repeat module as described more specifically in any of the aspects and embodiments herein.

In one embodiment, the recombinant binding protein of the invention comprises two or three ankyrin repeat domains with binding specificity for a target peptide-MHC complex, wherein each of said two or three ankyrin repeat domains independently comprises an amino acid sequence as described more specifically in any of the aspects and embodiments herein.

In one embodiment, the recombinant binding protein of the invention comprises exactly two ankyrin repeat domains with binding specificity for a target peptide-MHC complex, wherein each of said two ankyrin repeat domains independently comprises an amino acid sequence as described more specifically in any of the aspects and embodiments herein.

In one embodiment, the recombinant binding protein of the invention comprises three ankyrin repeat domains with binding specificity for a target peptide-MHC complex, wherein each of said three ankyrin repeat domains independently comprises an amino acid sequence as described more specifically in any of the aspects and embodiments herein.

In one embodiment, the recombinant binding protein of the invention comprises exactly three ankyrin repeat domains with binding specificity for a target peptide-MHC complex, wherein each of said three ankyrin repeat domains independently comprises an amino acid sequence as described more specifically in any of the aspects and embodiments herein.

In one embodiment, the recombinant binding protein of the invention comprises a single designed ankyrin repeat domain with binding specificity for a target peptide-MHC complex as described more specifically in any of the aspects and embodiments herein or comprises a combination of two, three, four, five or more designed ankyrin repeat domains with binding specificity for a target peptide-MHC complex as described more specifically in any of the aspects and embodiments herein.

In one aspect of the invention, the recombinant binding protein of the invention further comprises a binding agent.

In one particular embodiment, said binding agent has binding specificity for a protein expressed on the surface of an immune cell, preferably a T-cell, even more preferably a CD8+ cytotoxic T-cell. In a further embodiment, said protein expressed on the surface of a T-cell is a protein that is part of the T-cell receptor complex. As an example, in one particular embodiment, a recombinant binding protein of the invention further comprises a binding agent with binding specificity for cluster of differentiation 3 (CD3).

In the context of the present invention, a binding agent may be an antibody, an antibody mimetic, including a scaffold protein or a repeat protein, a designed repeat domain, preferably a designed ankyrin repeat domain, or any other suitable binding molecules known in the art. In one embodiment, said binding agent is an antibody. In another embodiment, said binding agent is a designed repeat domain, preferably a designed ankyrin repeat domain.

In one particular embodiment, said binding agent comprises or consists of an antibody with binding specificity for a protein expressed on the surface of an immune cell, preferably a T-cell, even more preferably a CD8+ cytotoxic T-cell. In a further embodiment, said protein expressed on the surface of a T-cell is a protein that is part of the T-cell receptor complex. As an example, in one particular embodiment, a recombinant binding protein of the invention further comprises an antibody with binding specificity for CD3.

In one particular embodiment, said binding agent comprises or consists of a designed repeat domain, preferably a designed ankyrin repeat domain, with binding specificity for a protein expressed on the surface of an immune cell, preferably a T-cell, even more preferably a CD8+ cytotoxic T-cell. In a further embodiment, said protein expressed on the surface of a T-cell is a protein that is part of the T-cell receptor complex. As an example, in one particular embodiment, a recombinant binding protein of the invention further comprises a designed ankyrin repeat domain with binding specificity for CD3.

In one embodiment, said binding agent is linked, conjugated, fused or otherwise physically attached to said pMHC-specific ankyrin repeat domain or said two, three or more pMHC-specific ankyrin repeat domains. In one embodiment, said binding agent is covalently linked to said pMHC-specific ankyrin repeat domain or said two, three or more pMHC-specific ankyrin repeat domains. In one embodiment, said binding agent is covalently linked to said pMHC-specific ankyrin repeat domain or said two, three or more pMHC-specific ankyrin repeat domains with a peptide linker. In one embodiment, said peptide linker is a proline-threonine rich peptide linker. In one embodiment, said peptide linker is the proline-threonine rich peptide linker of SEQ ID NO: 1 or 2. In one embodiment, said binding agent is covalently linked to said pMHC-specific ankyrin repeat domain or said two, three or more pMHC-specific ankyrin repeat domains with the proline-threonine rich peptide linker of SEQ ID NO: 1 or 2. In another embodiment, said peptide linker is a glycine-serine rich peptide linker. In one embodiment, said peptide linker is the glycine-serine rich peptide linker of SEQ ID NO: 3. In one embodiment, said binding agent is covalently linked to said pMHC-specific ankyrin repeat domain or said two, three or more pMHC-specific ankyrin repeat domains with the glycine-serine rich peptide linker of SEQ ID NO: 3.

It was surprisingly and unexpectedly found by the inventors of the present invention that the shorter the linker between said binding agent and said pMHC-specific ankyrin repeat domain (or said two, three or more pMHC-specific ankyrin repeat domains) the higher the potency of the construct in a T cell engager format (see Example 5). Thus, in a further preferred embodiment, the amino acid sequence of the peptide linker described herein has a length of less than 38 amino acids, preferably of at most 37, more preferably at most 24, even more preferably at most 18, even more preferably at most 11, most preferably at most 6 amino acids. In another preferred embodiment, the amino acid sequence of said peptide linker has a length from 1 to 37, from 1 to 24, from 1 to 23, from 1 to 18, from 1 to 17, from 1 to 11, from 1 to 10, from 1 to 6, from 6 to 37, from 6 to 24, from 6 to 23, from 6 to 18, from 6 to 17, or from 6 to 11 amino acid residues. Preferably, said peptide linker is a proline-threonine-rich peptide linker. In a further preferred embodiment, the amino acid sequence of said peptide linker has a length of less than 18 amino acids and of at least 1 amino acid, i.e. the peptide linker has a length from 1 to 17 amino acids. In one embodiment, said linker has the amino acid sequence as provided in any one of SEQ ID NOs: 1 and 74 to 77. In a preferred embodiment said peptide linker has the amino acid sequence as provided in SEQ ID NO: 75. In a more preferred embodiment said peptide linker has the amino acid sequence as provided in SEQ ID NO: 76. In a most preferred embodiment said peptide linker has the amino acid sequence as provided in SEQ ID NO: 77.

In one aspect of the invention, the recombinant binding protein of the invention, wherein said binding protein comprises one, two, three or more pMHC-specific ankyrin repeat domains as described more specifically in any of the aspects and embodiments herein, and wherein said binding protein further comprises a CD3-specific binding agent, is capable of facilitating infection- or tumor-localized activation of T cells. In such instances, the recombinant binding protein of the invention may be used in a T-cell engager format to locally activate T-cells against target peptide-MHC complex presenting tumor cells and/or target peptide-MHC complex presenting infectious cells.

In one aspect of the invention, the recombinant binding protein of the invention is capable of inhibiting recognition of said first target peptide-MHC complex and/or, if said binding protein comprises said second repeat domain, of said second target peptide-MHC complex by T-cell receptors. In other words, the recombinant binding protein of the invention is capable of inhibiting the ability of the immune system to recognize the target peptide-MHC complex(es). In such instances, the recombinant binding protein of the invention may be used for the treatment of an autoimmune disease. Measuring the recognition of a target peptide-MHC complex by T-cell receptors can be performed by any suitable methods known to the skilled person in the art or by suitable T-cell activation assays.

In one embodiment, the recombinant binding protein of the invention further comprises a polypeptide tag. In the context of the present invention, a polypeptide tag is an amino acid sequence attached to a polypeptide/protein, wherein said amino acid sequence is useful for the purification, detection, or targeting of said polypeptide/protein, or wherein said amino acid sequence improves the physicochemical behavior of the polypeptide/protein, or wherein said amino acid sequence possesses an effector function. The individual polypeptide tags of a binding protein may be connected to other parts of the binding protein directly or via peptide linkers. Polypeptide tags are all well known in the art and are fully available to the person skilled in the art. Examples of polypeptide tags are small polypeptide sequences, for example, His, HA, myc, FLAG, or Strep-tags, or polypeptides such as enzymes (for example alkaline phosphatase), which allow the detection of said polypeptide/protein, or polypeptides which can be used for targeting (such as immunoglobulins or fragments thereof) and/or as effector molecules.

In one embodiment, the recombinant binding protein of the invention further comprises a peptide linker. In the context of the present invention, a peptide linker is an amino acid sequence, which is able to link, for example, two protein domains, a polypeptide tag and a protein domain, a protein domain and a non-proteinaceous compound or polymer such as polyethylene glycol, a protein domain and a biologically active molecule, a protein domain and a T-cell-specific ankyrin repeat domain, or two sequence tags. Peptide linkers are known to the person skilled in the art. A list of examples is provided in the description of patent application WO2002/020565. Particular examples of such linkers are glycine-serine-linkers and proline-threonine-linkers of variable lengths. Examples of a glycine-serine-linker are the amino acid sequence GS and the amino acid sequence of SEQ ID NO: 3, and examples of a proline-threonine-linker are the amino acid sequences of SEQ ID NO: 1 and SEQ ID NO: 2.

In another aspect, the invention relates to a nucleic acid encoding the amino acid sequence of the designed repeat domain of the present invention or of the pMHC-specific recombinant binding protein of the present invention. In one embodiment, the invention relates to a nucleic acid encoding the amino acid sequence of the pMHC-specific recombinant binding protein of the present invention. In one embodiment, the invention relates to a nucleic acid encoding the amino acid sequence of the designed repeat domain of the present invention. In one embodiment, the invention relates to a nucleic acid encoding an amino acid sequence selected from the group consisting of SEQ ID NO: 16 to 26. In one embodiment, the invention relates to a nucleic acid encoding an amino acid sequence selected from the group consisting of SEQ ID NO: 16 to 18 and 20 to 26. In one embodiment, the invention relates to a nucleic acid encoding an amino acid sequence selected from the group consisting of SEQ ID NO: 17, 21, 22, 23 and 26. In one embodiment, the invention relates to a nucleic acid encoding an amino acid sequence selected from the group consisting of SEQ ID NO: 21 and 23. In one embodiment, the invention relates to a nucleic acid encoding the amino acid sequence of SEQ ID NO: 17. In one embodiment, the invention relates to a nucleic acid encoding the amino acid sequence of SEQ ID NO: 21. In one embodiment, the invention relates to a nucleic acid encoding the amino acid sequence of SEQ ID NO: 22. In one embodiment, the invention relates to a nucleic acid encoding the amino acid sequence of SEQ ID NO: 23. In one embodiment, the invention relates to a nucleic acid encoding the amino acid sequence of SEQ ID NO: 26. Furthermore, the invention relates to vectors comprising any nucleic acid of the invention. Nucleic acids are well known to the skilled person in the art. In the examples, nucleic acids were used to produce designed ankyrin repeat domains or recombinant binding proteins of the invention in E. coli.

In another aspect, the invention relates to a pharmaceutical composition comprising the pMHC-specific recombinant binding protein of the present invention and/or the nucleic acid of the present invention, and optionally a pharmaceutically acceptable carrier and/or diluent.

Pharmaceutically acceptable carriers and/or diluents are known to the person skilled in the art and are explained in more detail below. Even further, a diagnostic composition is provided comprising one or more of the above-mentioned recombinant binding proteins and/or nucleic acids, in particular recombinant binding proteins of the present invention.

A pharmaceutical composition comprises a recombinant binding protein and/or a nucleic acid, preferably a recombinant binding protein and/or a nucleic acid as described more specifically in any of the aspects or embodiments herein, and a pharmaceutically acceptable carrier, excipient or stabilizer, for example as described in Remington's Pharmaceutical Sciences 16^{th} edition, Osol, A. Ed., 1980.

Suitable carriers, excipients or stabilizers known to one of skill in the art include, for example, saline, Ringer's solution, dextrose solution, Hank's solution, fixed oils, ethyl oleate, 5% dextrose in saline, substances that enhance isotonicity and chemical stability, buffers and preservatives. Other suitable carriers include any carrier that does not itself induce the production of antibodies harmful to the individual receiving the composition such as proteins, polysaccharides, polylactic acids, polyglycolic acids, polymeric amino acids and amino acid copolymers. A pharmaceutical composition may also be a combination formulation, comprising an additional active agent, such as an anti-cancer agent or an anti-angiogenic agent, or an additional bioactive compound.

The formulations to be used for in vivo administration must be aseptic or sterile. This is readily accomplished by filtration through sterile filtration membranes.

In one embodiment, a pharmaceutical composition comprises at least one recombinant binding protein as described herein and a detergent, such as, e.g., a nonionic detergent, a buffer, such as, e.g., phosphate buffer, and a sugar, such as, e.g., sucrose. In one embodiment, such a composition comprises recombinant binding proteins as described above and PBS.

In another aspect, the invention provides a method of infection-localized activation of immune cells, preferably T-cells, in a mammal, preferably a human, the method comprising the step of administering to said mammal the pMHC-specific recombinant binding protein of the invention comprising a first repeat domain with binding specificity for a first target peptide-MHC complex and optionally comprising a second repeat domain with binding specificity for a second target peptide-MHC complex and further comprising a binding agent with binding specificity for a protein expressed on the surface of immune cells, preferably T-cells, wherein said first target peptide and/or, if said binding protein comprises said second repeat domain, said second target peptide is derived from a protein of an infectious agent. In one embodiment, said infection is a viral infection. In one embodiment, said first and/or, if present, said second target peptide is derived from a protein of a viral infectious agent. In one embodiment, said first and/or, if present, said second target peptide is derived from a virus-specific protein. In one embodiment, said first and/or, if present, said second target peptide is derived from HBcAg. In one embodiment, said first and/or, if present, said second target peptide has the amino acid sequence of SEQ ID NO: 51. In one embodiment, said binding agent has binding specificity for a protein expressed on the surface of an immune cell, preferably a T-cell, even more preferably a CD8+ cytotoxic T-cell. In one embodiment, said binding agent is an antibody with binding specificity for a protein expressed on the surface of an immune cell, preferably a T-cell, even more preferably a CD8+ cytotoxic T-cell. In one embodiment, said binding agent is a designed repeat domain with binding specificity for a protein expressed on the surface of an immune cell, preferably a T-cell, even more preferably a CD8+ cytotoxic T-cell. In one embodiment, said binding agent is a designed ankyrin repeat domain with binding specificity for a protein expressed on the surface of an immune cell, preferably a T-cell, even more preferably a CD8+ cytotoxic T-cell. In a preferred embodiment, said protein expressed on the surface of an immune cell is a protein that is part of the T-cell receptor complex, such as, e.g., CD3. In one embodiment, said first ankyrin repeat domain and/or, if present, said second ankyrin repeat are independently designed ankyrin repeat domains with binding specificity for a target peptide-MHC complex as described more specifically in any of the aspects and embodiments herein.

Further provided is the pMHC-specific recombinant binding protein of the present invention comprising a binding agent with binding specificity for a protein expressed on the surface of an immune cell, preferably a T-cell, even more preferably a CD8+ cytotoxic T-cell, the nucleic acid of the invention, or the pharmaceutical composition of the invention for use in a method of infection-localized activation of immune cells as described herein.

In another aspect, the invention provides a method of treating a medical condition, the method comprising the step of administering to a patient in need thereof a therapeutically effective amount of the pMHC-specific recombinant binding protein of the invention, the nucleic acid of the invention or the pharmaceutical composition of the invention.

Further provided is the pMHC-specific recombinant binding protein, the nucleic acid, or the pharmaceutical composition of the invention for use in a method of treating a medical condition as described herein.

In another aspect, the invention provides a method of diagnosing a medical condition in a mammal, preferably a human, the method comprising the step of:

(i) contacting a cell or tissue sample obtained from said mammal with the recombinant binding protein of the invention; and

(ii) detecting specific binding of said binding protein to said cell or tissue sample. In one embodiment, said tissue is tumor tissue.

In the context of the invention, the terms “medical condition”, “disease” and “disorder” are used interchangeably and include but are not limited to cancer, infectious disease, and autoimmune disease. In one preferred embodiment, said medical condition is a cancer, an infectious disease, preferably a viral infectious disease, or an autoimmune disease. In one embodiment, said medical condition is a cancer. In one embodiment, such cancer is selected from the group consisting of epithelial malignancies (primary and metastatic), including but not limited to lung, colorectal, gastric, bladder, ovarian and breast carcinomas, blood cell malignancies, including but not limited to leukemia, lymphoma, and myeloma, sarcomas, including but not limited to bone and soft tissue sarcomas, and melanoma. In one preferred embodiment, such cancer is selected from the group consisting of liposarcoma, neuroblastoma, synovial sarcoma, melanoma and ovarian cancer. In another most preferred embodiment, such cancer is selected from the group consisting of melanoma, lung cancer, liver cancer, stomach cancer, skin cancer, neuroblastoma, soft tissue sarcoma, bladder cancer, testicular cancer and ovarian cancer. In one embodiment, said medical condition is an infectious disease, preferably a viral infectious disease. In one preferred embodiment, such infectious disease is a viral infection caused by hepatitis B virus (HBV). In another preferred embodiment, such infectious disease is a viral infection caused by Epstein-Barr virus (EBV). In one embodiment, said medical condition is an autoimmune disease. In one preferred embodiment, such autoimmune disease is selected from the group consisting of systemic lupus erythematosus, rheumatoid arthritis and type I diabetes.

In one embodiment, the invention relates to the use of the pharmaceutical composition, nucleic acid or recombinant binding protein according to the present invention for the treatment of a disease. For that purpose, the pharmaceutical composition, the nucleic acid or the recombinant binding protein according to the present invention is administered, to a patient in need thereof, in a therapeutically effective amount. Administration may include topical administration, oral administration, and parenteral administration. The typical route of administration is parenteral administration. In parental administration, the pharmaceutical composition of this invention will be formulated in a unit dosage injectable form such as a solution, suspension or emulsion, in association with the pharmaceutically acceptable excipients as defined above. The dosage and mode of administration will depend on the individual to be treated and the particular disease.

Further, any of the above-mentioned pharmaceutical compositions or recombinant binding proteins is considered for the treatment of a disorder.

In one embodiment, said recombinant binding protein or pharmaceutical composition of the invention as described herein is applied intravenously. For parenteral application, said recombinant binding protein or pharmaceutical composition can be injected as bolus injection or by slow infusion at a therapeutically effective amount.

In one embodiment, the invention relates to a method of treatment of a medical condition, the method comprising the step of administering, to a patient in need of such a treatment, a therapeutically effective amount of the recombinant binding protein, nucleic acid, or pharmaceutical composition of the invention. In one embodiment, the invention relates to the use of the recombinant binding protein, nucleic acid, or pharmaceutical composition of the present invention for the treatment of a medical condition. In one embodiment, the invention relates to the recombinant binding protein, nucleic acid, or pharmaceutical composition of the invention for use in the treatment of a medical condition. In one embodiment, the invention relates to the use of the pharmaceutical composition, recombinant binding protein, or nucleic acid of the invention, as a medicament for the treatment of a medical condition. In one embodiment, the invention relates to the use of the pharmaceutical composition, recombinant binding protein, or nucleic acid of the invention, for manufacturing of a medicament. In one embodiment, the invention relates to the use of the pharmaceutical composition, recombinant binding protein, or nucleic acid of the invention, for manufacturing of a medicament for the treatment of a medical condition. In one embodiment, the invention relates to a process for the manufacturing of a medicament for the treatment of a medical condition, wherein the pharmaceutical composition, recombinant binding protein, or nucleic acid of the invention is an active ingredient of the medicament. In one embodiment, the invention relates to a process of treatment of a medical condition using the pharmaceutical composition, recombinant binding protein, or nucleic acid of the invention.

In a further embodiment, the invention relates to the use of the recombinant binding protein, nucleic acid or pharmaceutical composition of the invention for the manufacture of a medicament that is used for the treatment of a medical condition, preferably an infectious disease, more preferably a viral infectious disease.

In one embodiment, the invention relates to a recombinant binding protein comprising any of the above mentioned designed ankyrin repeat domains for therapeutic purposes.

In one aspect, the invention relates to a kit comprising the recombinant binding protein of the invention. In one embodiment, the invention relates to a kit comprising a nucleic acid encoding the recombinant binding protein of the invention. In one embodiment, the invention relates to a kit comprising the pharmaceutical composition of the invention. In one embodiment, the invention relates to a kit comprising the recombinant binding protein of the invention, and/or a nucleic acid encoding the recombinant binding protein of the invention, and/or the pharmaceutical composition of the invention. In one embodiment, the invention relates to a kit comprising a recombinant binding protein comprising one or more peptide-MHC-specific ankyrin repeat domains of the invention, for example one or more peptide-MHC-specific ankyrin repeat domains independently having the amino acid sequence of SEQ ID NO: 17, SEQ ID NO: 21, SEQ ID NO: 22, SEQ ID NO: 23 or SEQ ID NO: 26, and/or a nucleic acid encoding a recombinant binding protein comprising one or more peptide-MHC-specific ankyrin repeat domains of the invention, for example one or more peptide-MHC-specific ankyrin repeat domains independently having the amino acid sequence of SEQ ID NO: 17, SEQ ID NO: 21, SEQ ID NO: 22, SEQ ID NO: 23 or SEQ ID NO: 26, and/or a pharmaceutical composition comprising a recombinant binding protein comprising one or more peptide-MHC-specific ankyrin repeat domains of the invention, for example one or more peptide-MHC-specific ankyrin repeat domains independently having the amino acid sequence of SEQ ID NO: 17, SEQ ID NO: 21, SEQ ID NO: 22, SEQ ID NO: 23 or SEQ ID NO: 26.

In another aspect, the invention provides a method of targeting infected cells in a patient having a viral infectious disease for destruction of the infected cells, the method comprising the step of administering to the patient a therapeutically effective amount of the binding protein, nucleic acid or pharmaceutical composition of the invention, wherein said first target peptide and/or, if said binding protein comprises said second repeat domain, said second target peptide is derived from a protein expressed in the infected cells, preferably a virus-specific protein. In one embodiment, said binding protein further comprises a toxic agent capable of killing an infected cell.

Further provided is the recombinant binding protein, nucleic acid, or pharmaceutical composition of the invention for use in a method of targeting infected cells in a patient having a viral infectious disease for destruction of the infected cells, wherein said first target peptide and/or, if said binding protein comprises said second repeat domain, said second target peptide is derived from a protein expressed in the infected cells, preferably a virus-specific protein. In one embodiment, said binding protein further comprises a toxic agent capable of killing an infected cell.

In a preferred embodiment, the medical condition to be treated by the method of treatment of the invention or by the binding protein, the nucleic acid or the pharmaceutical composition of the invention is a viral infectious disease caused by hepatitis B virus.

This specification refers to a number of amino acid sequences, nucleic acid sequences and SEQ ID NOs that are disclosed in the appended Sequence Listing, which is herewith incorporated by reference in its entirety.

### Definitions

Unless defined otherwise herein, all technical and scientific terms used herein shall have the meanings that are commonly understood by those of ordinary skill in the art to which the present invention belongs.

In the context of the present invention, the term “collection” refers to a population comprising at least two different entities or members. Preferably, such a collection comprises at least 10⁵, more preferably more than 10⁷, and most preferably more than 10⁹ different members. A “collection” may as well be referred to as a “library” or a “plurality”.

The term “nucleic acid molecule” refers to a polynucleotide molecule, which may be a ribonucleic acid (RNA) or deoxyribonucleic acid (DNA) molecule, either single stranded or double stranded, and includes modified and artificial forms of DNA or RNA. A nucleic acid molecule may either be present in isolated form, or be comprised in recombinant nucleic acid molecules or vectors.

In the context of the present invention the term “protein” refers to a molecule comprising a polypeptide, wherein at least part of the polypeptide has, or is able to acquire, a defined three-dimensional arrangement by forming secondary, tertiary, and/or quaternary structures within a single polypeptide chain and/or between multiple polypeptide chains. If a protein comprises two or more polypeptide chains, the individual polypeptide chains may be linked non-covalently or covalently, e.g. by a disulfide bond between two polypeptides. A part of a protein, which individually has, or is able to acquire, a defined three-dimensional arrangement by forming secondary and/or tertiary structure, is termed “protein domain”. Such protein domains are well known to the practitioner skilled in the art.

The term “recombinant” as used in recombinant protein, recombinant polypeptide and the like, means that said protein or polypeptide is produced by the use of recombinant DNA technologies well known to the practitioner skilled in the art. For example, a recombinant DNA molecule (e.g. produced by gene synthesis) encoding a polypeptide can be cloned into a bacterial expression plasmid (e.g. pQE30, QIAgen), yeast expression plasmid, mammalian expression plasmid, or plant expression plasmid, or a DNA enabling in vitro expression. If, for example, such a recombinant bacterial expression plasmid is inserted into appropriate bacteria (e.g. Escherichia coli), these bacteria can produce the polypeptide(s) encoded by this recombinant DNA. The correspondingly produced polypeptide or protein is called a recombinant polypeptide or recombinant protein.

In the context of the present invention, the term “binding protein” refers to a protein comprising a binding domain. A binding protein may also comprise two, three, four, five or more binding domains. Preferably, said binding protein is a recombinant binding protein. More preferably, the binding proteins of the instant invention comprise an ankyrin repeat domain with binding specificity for a pMHC complex.

Furthermore, any such binding protein may comprise additional polypeptides (such as e.g. polypeptide tags, peptide linkers, fusion to other proteinaceous domains with binding specificity, cytokines, hormones, or antagonists), or chemical modifications (such as coupling to polyethylene-glycol, toxins, small molecules, antibiotics and alike) well known to the person skilled in the art. In some embodiments, the binding protein further comprises a binding agent with binding specificity for a protein expressed on the surface of an immune cell. In some embodiments, the binding protein further comprises a toxic agent capable of killing a tumor cell and/or an infected cell. Examples of toxic agents include, but are not limited to, vinblastine, doxorubicin, topoisomerase I inhibitors, calicheamicins, duocarmycin-hydroxybenzamide-azaindole (DUBA), pyrrolobenzodiazepine dimers (PBD), and derivatives of the microtubule inhibitor family, such as auristatin and maytansine, e.g. monomethyl auristatin E (MMAE), monomethyl auristatin F (MMAF), drug maytansinoid 1 (DM1) and drug maytansinoid 4 (DM4).

The term “binding domain” means a protein domain exhibiting binding specificity for a target. Preferably, said binding domain is a recombinant binding domain.

The term “target” refers to an individual molecule such as a nucleic acid molecule, a peptide, polypeptide or protein, a carbohydrate, or any other naturally occurring molecule, including any part of such individual molecule, or to complexes of two or more of such molecules, or to a whole cell or a tissue sample, or to any non-natural compound. Preferably, a target is a naturally occurring or non-natural polypeptide or protein, or a polypeptide or protein containing chemical modifications, for example, naturally occurring or non-natural phosphorylation, acetylation, or methylation. In the context of the present invention, peptide-MHC complexes and peptide-MHC presenting cells and tissues are targets of pMHC-specific binding proteins. Furthermore, CD3 and CD3-expressing cells, such as, e.g., T-cells, are targets of pMHC-specific binding proteins of the invention that further comprise a binding agent with binding specificity for a protein expressed on the surface of an immune cell, such as CD3.

In the context of the present invention, the term “polypeptide” relates to a molecule consisting of a chain of multiple, i.e. two or more, amino acids linked via peptide bonds. Preferably, a polypeptide consists of more than eight amino acids linked via peptide bonds. The term “polypeptide” also includes multiple chains of amino acids, linked together by S—S bridges of cysteines. Polypeptides are well-known to the person skilled in the art.

Patent application WO2002/020565 and Forrer et al., 2003 (Forrer, P., Stumpp, M. T., Binz, H. K., Pluckthun, A., 2003. FEBS Letters 539, 2-6), contain a general description of repeat protein features and repeat domain features, techniques and applications. The term “repeat protein” refers to a protein comprising one or more repeat domains. Preferably, a repeat protein comprises one, two, three, four, five or six repeat domains. Furthermore, said repeat protein may comprise additional non-repeat protein domains, polypeptide tags and/or peptide linkers. The repeat domains can be binding domains.

The term “repeat domain” refers to a protein domain comprising two or more consecutive repeat modules as structural units, wherein said repeat modules have structural and sequence homology. Preferably, a repeat domain also comprises an N-terminal and/or a C-terminal capping module. For clarity, a capping module can be a repeat module. Such repeat domains, repeat modules, and capping modules, sequence motives, as well as structural homology and sequence homology are well known to the practitioner in the art from examples of ankyrin repeat domains (Binz et al., J. Mol. Biol. 332, 489-503, 2003; Binz et al., 2004, loc. cit.; WO2002/020565; WO2012/069655), leucine-rich repeat domains (WO2002/020565), tetratricopeptide repeat domains (Main, E. R., Xiong, Y., Cocco, M. J., D'Andrea, L., Regan, L., Structure 11(5), 497-508, 2003), and armadillo repeat domains (WO2009/040338). It is further well known to the practitioner in the art, that such repeat domains are different from proteins comprising repeated amino acid sequences, where every repeated amino acid sequence is able to form an individual domain (for example FN3 domains of Fibronectin).

The term “ankyrin repeat domain” refers to a repeat domain comprising two or more consecutive ankyrin repeat modules as structural units, wherein said ankyrin repeat modules have structural and sequence homology.

The term “designed” as used in designed repeat protein, designed repeat domain and the like refers to the property that such repeat proteins and repeat domains, respectively, are man-made and do not occur in nature. The binding proteins of the instant invention are designed repeat proteins and they comprise at least one designed repeat domain. Preferably, the designed repeat domain is a designed ankyrin repeat domain.

The term “target interaction residues” refers to amino acid residues of a repeat module, which contribute to the direct interaction with a target.

The terms “framework residues” or “framework positions” refer to amino acid residues of a repeat module, which contribute to the folding topology, i.e. which contribute to the fold of said repeat module or which contribute to the interaction with a neighboring module. Such contribution may be the interaction with other residues in the repeat module, or the influence on the polypeptide backbone conformation as found in α-helices or β-sheets, or the participation in amino acid stretches forming linear polypeptides or loops.

Such framework and target interaction residues may be identified by analysis of the structural data obtained by physicochemical methods, such as X-ray crystallography, NMR and/or CD spectroscopy, or by comparison with known and related structural information well known to practitioners in structural biology and/or bioinformatics.

The term “repeat modules” refers to the repeated amino acid sequence and structural units of the designed repeat domains, which are originally derived from the repeat units of naturally occurring repeat proteins. Each repeat module comprised in a repeat domain is derived from one or more repeat units of a family or subfamily of naturally occurring repeat proteins, preferably the family of ankyrin repeat proteins.

Furthermore, each repeat module comprised in a repeat domain may comprise a “repeat sequence motif” deduced from homologous repeat modules obtained from repeat domains selected on a target and having the same target specificity.

Accordingly, the term “ankyrin repeat module” refers to a repeat module, which is originally derived from the repeat units of naturally occurring ankyrin repeat proteins. Ankyrin repeat proteins are well known to the person skilled in the art.

Repeat modules may comprise positions with amino acid residues which have not been randomized in a library for the purpose of selecting target-specific repeat domains (“non-randomized positions” or “fixed positions” used interchangeably herein) and positions with amino acid residues which have been randomized in the library for the purpose of selecting target-specific repeat domains (“randomized positions”). The non-randomized positions comprise framework residues. The randomized positions comprise target interaction residues. “Have been randomized” means that two or more amino acids were allowed at an amino acid position of a repeat module, for example, wherein any of the usual twenty naturally occurring amino acids were allowed, or wherein most of the twenty naturally occurring amino acids were allowed, such as amino acids other than cysteine, or amino acids other than glycine, cysteine and proline. For the purpose of this patent application, amino acid residues 3, 4, 6, 14 and 15 of SEQ ID NOs: 27 to 50 are randomized positions of the ankyrin repeat modules of the instant invention.

The term “repeat sequence motif” refers to an amino acid sequence, which is deduced from one or more repeat modules. Preferably, said repeat modules are from repeat domains having binding specificity for the same target. Such repeat sequence motifs comprise framework residue positions and target interaction residue positions. Said framework residue positions correspond to the positions of framework residues of the repeat modules. Likewise, said target interaction residue positions correspond to the positions of target interaction residues of the repeat modules. Repeat sequence motifs comprise non-randomized positions and randomized positions.

The term “repeat unit” refers to amino acid sequences comprising sequence motifs of one or more naturally occurring proteins, wherein said “repeat units” are found in multiple copies, and exhibit a defined folding topology common to all said motifs determining the fold of the protein. Examples of such repeat units include leucine-rich repeat units, ankyrin repeat units, armadillo repeat units, tetratricopeptide repeat units, HEAT repeat units, and leucine-rich variant repeat units.

The term “binding specificity”, “has binding specificity for a target”, “specifically binding to a target”, “binding to a target with high specificity”, “specific for a target” or “target specificity” and the like means that a binding protein or binding domain binds in PBS to a target with a lower dissociation constant (i.e. it binds with higher affinity) than it binds to an unrelated protein such as the E. coli maltose binding protein (MBP). Preferably, the dissociation constant (“K_{D}”) in PBS for the target is at least 10²; more preferably, at least 10³; more preferably, at least 10⁴; or more preferably, at least 10⁵ times lower than the corresponding dissociation constant for MBP. Methods to determine dissociation constants of protein-protein interactions, such as surface plasmon resonance (SPR) based technologies (e.g. SPR equilibrium analysis) or isothermal titration calorimetry (ITC) are well known to the person skilled in the art. The measured K_{D} values of a particular protein-protein interaction can vary if measured under different conditions (e.g., salt concentration, pH). Thus, measurements of K_{D} values are preferably made with standardized solutions of protein and a standardized buffer, such as PBS. A typical and preferred determination of dissociation constants (K_{D}) of the inventive recombinant binding proteins with binding specificity for pMHC by Surface Plasmon Resonance (SPR) analysis is described in Example 2.

The term “about” means the mentioned value +/−20%; for example “about 50” shall mean 40 to 60.

The term “PBS” means a phosphate buffered water solution containing 137 mM NaCl, 10 mM phosphate and 2.7 mM KCl and having a pH of 7.4.

The major histocompatibility complex (MHC) is a group of genes that code for proteins found on the surfaces of cells that help the immune system recognize foreign substances. The proteins encoded by the major histocompatibility complex, or complexes of such proteins, are called “MHC proteins” or “MHC molecules”. MHC proteins are found in all higher vertebrates. Most notable are the MHC class I and class II glycoproteins that present peptides to an immune cell receptor, such as the T-cell receptor. In humans, the major histocompatibility complex is also called the human leukocyte antigen (HLA) system.

The term “MHC” as used herein refers to the major histocompatibility complex, preferably the major histocompatibility complex of a mammal, even more preferably the major histocompatibility complex of a human. The term “HLA” as used herein refers to the human leukocyte antigen system. An MHC molecule displays a peptide and presents it to the immune system of the vertebrate. The terms “peptide antigen” and “MHC peptide antigen” are used interchangeably herein and refer to an MHC ligand that can bind in the peptide binding groove of an MHC molecule. A peptide antigen typically has between 8 and 25 amino acids that are linked via peptide bonds. The MHC peptide antigen can be either a self or a non-self peptide. The peptide antigen can typically be presented to the immune system by the MHC molecule. MHC-class I molecules typically present the peptide antigen to CD8 positive T-cells whereas MHC-class II molecules typically present the peptide antigen to CD4 positive T-cells. In the context of the present invention, a peptide antigen that is specifically bound by a binding protein of the invention, when the peptide antigen is bound to a MHC molecule, is also called a “target peptide”. Accordingly, the terms “peptide-MHC complex”, “pMHC complex”, “peptide-MHC”, and “pMHC” are used interchangeably in the present application, and refer to a complex formed by the binding of a peptide antigen to an MHC molecule. Preferably, the MHC molecule is an MHC-class I molecule. Also preferably, the peptide is a peptide derived from a protein expressed in a tumor cell, a peptide derived from a protein of an infectious agent, e.g., a viral infectious agent, or a peptide derived from a protein associated with an autoimmune disorder. In one preferred embodiment, said protein expressed in a tumor cell is a tumor-specific protein.

The term “tumor-specific protein” means a protein that is expressed in tumor cells and that is not expressed or is expressed only at a lower level in many or all non-tumorigenic tissues or that is expressed only in a limited number of non-tumorigenic tissues in addition to the tumor tissue. Preferably, a tumor-specific protein has the capacity to elicit an immune response that is cancer-specific. Examples of tumor-specific proteins are known to the skilled person in the art and include but are not limited to MAGE-A1, MAGE-A3, MAGE-A4, NY-ESO-1, PRAME, CT83, SSX2 and the like.

In one preferred embodiment, said protein of an infectious agent or said protein expressed in an infected cell is a virus-specific protein.

The term “viral-specific protein” means a protein that is expressed from a viral genome in a virus-infected cells and that is not expressed in non-infected host cells. Examples of virus-specific proteins are known to the skilled person in the art and include but are not limited to EBNA-1, EBNA-2, EBNA-3, LMP-1, LMP-2, NSP1, NSP2, NSP4, NSP5, NSP6, E1, E2, HBx, HBsAg, HBcAg, and the like. Examples of proteins associated with an autoimmune disorder are known to the skilled person in the art and include but are not limited to Carboxypeptidase H, Chromogranin A, Insulin, β-arrestin, Aquaporin-4, Citrullinated protein and the like.

A “target peptide-MHC complex” refers to a peptide-MHC complex, in which the peptide antigen is a target peptide.

More preferably, the peptide antigen is derived from a protein of an infectious agent, even more preferably a protein of a viral infectious agent. Even more preferably, the viral infectious agent is Hepatitis B virus (HBV). Most preferably, said protein of HBV is a virus-specific protein.

Most preferably, the peptide antigen is derived from the hepatitis B core antigen (HBcAg). Preferably, the peptide antigen derived from HBcAg is the sequence 18-27 of the HBV core antigen, abbreviated as “HBVc18” or “c18 peptide” herein, and has the amino acid sequence FLPSDFFPSV (SEQ ID NO: 51).

The term “peptide derived from” as used in “peptide derived from a tumor-specific protein”, “peptide derived from an intracellular protein”, “peptide derived from a protein of an infectious agent”, “peptide derived from a protein associated with an autoimmune disorder” and the like refers to a molecular fraction or a peptide fragment of the respective protein. Such a peptide fragment may result from the degradation of normal or pathogenic proteins in order to be presented, i.e. displayed, in association with an MHC molecule on the surface of a cell, for recognition by certain lymphocytes such as T cells. The presented peptide can be either self or non-self, and an organism's immune system can normally distinguish between self and non-self, thus preventing an organism's immune system from targeting its own cells. Alternatively, such a peptide fragment may be produced by methods known in the art. Preferably, the peptide is derived from an intracellular protein.

The term “binding agent” refers to any molecule capable of specifically binding a target molecule and includes, for example, antibodies, antibody fragments, aptamers, peptides (e.g., Williams et al., J Biol Chem 266:5182-5190 (1991)), antibody mimics, repeat proteins, e.g. designed anykrin repeat proteins, receptor proteins and any other naturally occurring interaction partners of the target molecule, and can comprise natural proteins and proteins modified or genetically engineered, e.g., to include non-natural residues and/or to lack natural residues.

The term “negative selection” or “de-selection” of a binding domain means the selective removal of a binding domain, which is not or less suitable for the purpose of the invention, from a collection of binding domains. In the context of the present invention, negative selection or de-selection preferably means the selective removal, from a collection of designed repeat domains, of unwanted designed repeat domain(s) with binding specificity for a peptide-MHC complex other than the target peptide-MHC complex or with binding specificity for the common HLA-A scaffold of different pMHC complexes. Methods for negatively selecting or de-selecting an unwanted member of a collection, such as, e.g., an unwanted binding domain of a collection of binding domains, are known to the skilled person of the art.

The term “peptide-MHC presenting cells” as used herein refers to any cells capable of expressing or displaying a peptide-MHC on the cell surface. This includes, but is not limited to, tumor cells, infected cells, cells associated with autoimmune disorders, classical antigen-presenting cells such as dendritic cells, macrophages, and B cells, as well as cells that have been made to display MHC molecules bound to exogenously administered peptides, such as peptide-pulsed T2 cells.

The term “CD3-expressing cells” as used herein refers to any cells expressing CD3 (cluster of differentiation 3) on the cell surface, including, but not limited, to T cells such as cytotoxic T cells (CD8+ T cells) and T helper cells (CD4+ T cells).

The term “tumor-localized activation of T cells” means that T cells are activated preferentially in tumor tissue as compared to a non-tumor tissue.

The term “infection-localized activation of T cells” means that T cells are activated preferentially in an infected tissue as compared to a non-infected tissue.

The term “medical condition” (or “disorder” or “disease”) includes but is not limited to autoimmune disorders, inflammatory disorders, retinopathies (particularly proliferative retinopathies), neurodegenerative disorders, infectious diseases, metabolic diseases, and neoplastic diseases. Any of the recombinant binding proteins described herein may be used for the preparation of a medicament for the treatment of such a disorder, particularly a disorder selected from the group comprising: an autoimmune disorder, an inflammatory disorder, an immune disorder, an infectious disease (e.g. a viral or bacterial infectious disease) and a neoplastic disease. A “medical condition” may be one that is characterized by inappropriate cell proliferation. A medical condition may be a hyperproliferative condition. The invention particularly relates to a method of treating a medical condition, the method comprising the step of administering, to a patient in need of such treatment, a therapeutically effective amount of a recombinant binding protein or pharmaceutical composition of the invention. In a preferred embodiment, said medical condition is a neoplastic disease. The term “neoplastic disease”, as used herein, refers to an abnormal state or condition of cells or tissue characterized by rapidly proliferating cell growth or neoplasm. In one embodiment, said medical condition is a malignant neoplastic disease. In one embodiment, said medical condition is a cancer. In another preferred embodiment, said medical condition is an infectious disease. In another preferred embodiment, said medical condition is an autoimmune disease. The term “therapeutically effective amount” means an amount that is sufficient to produce a desired effect on a patient.

The term “antibody” means not only intact antibody molecules, but also any fragments and variants of antibody molecules that retain immunogen-binding ability. Such fragments and variants are also well known in the art and are regularly employed both in vitro and in vivo. Accordingly, the term “antibody” encompasses intact immunoglobulin molecules, antibody fragments such as, e.g., Fab, Fab′, F(ab′)₂, and single chain V region fragments (scFv), bispecific antibodies, chimeric antibodies, humanized antibodies, antibody fusion polypeptides, and unconventional antibodies.

The terms “cancer” and “cancerous” are used herein to refer to or describe the physiological condition in mammals that is typically characterized by unregulated cell growth. Cancer encompasses solid tumors and liquid tumors, as well as primary tumors and metastases. A “tumor” comprises one or more cancerous cells. Solid tumors typically also comprise tumor stroma. Examples of cancer include, but are not limited to, primary and metastatic carcinoma, lymphoma, blastoma, sarcoma, myeloma, melanoma and leukemia, and any other epithelial and blood cell malignancies. More particular examples of such cancers include brain cancer, bladder cancer, breast cancer, ovarian cancer, kidney cancer, colorectal cancer, gastric cancer, head and neck cancer, lung cancer, pancreatic cancer, prostate cancer, malignant melanoma, osteosarcoma, soft tissue sarcoma, carcinoma, squameous cell carcinoma, clear cell kidney cancer, head/neck squamous cell carcinoma, lung adenocarcinoma, lung squamous cell carcinoma, non-small-cell lung cancer (NSCLC), renal cell carcinoma, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myelodysplastic syndrome (MDS), non-Hodgkin's lymphoma (NHL), Squamous Cell Carcinoma of the Head and Neck (SCCHN), chronic myelogenous leukemia (CML), small lymphocytic lymphoma (SLL), malignant mesothelioma, liposarcoma, neuroblastoma, or synovial sarcoma.

The term “therapeutically effective amount” refers to the amount sufficient to induce a desired biological, pharmacological, or therapeutic outcome in a subject. A therapeutically effective amount in the context of the invention means a sufficient amount of the binding protein to treat or prevent a disease or disorder at a reasonable benefit/risk ratio applicable to any medical treatment.

The term “treatment” or “treating” refers to both therapeutic treatment and prophylactic or preventative measures. Those in need of treatment include those who have already the disorder as well as those in which the disorder is to be prevented.

The term “mammal” for purposes of treatment refers to any animal classified as a mammal, including human, domestic and farm animals, nonhuman primates, and zoo, sports, or pet animals, such as dogs, horses, cats, cows, etc. The terms “autoimmune disease” and “autoimmune disorder” are used herein to refer to or describe disorders wherein the immune system of a mammal mounts a humoral or cellular immune response to the mammal's own tissue or to antigens that are not intrinsically harmful to the mammal, thereby producing tissue injury in such a mammal. Examples of autoimmune disorders are numerous and include, but are not limited to, systemic lupus erythematosus, rheumatoid arthritis and type I diabetes. Autoimmune diseases also include acute glomerulonephritis, Addison's disease, adult onset idiopathic hypoparathyroidism (AOIH), alopecia totalis, amyotrophic lateral sclerosis, ankylosing spondylitis, autoimmune aplastic anemia, autoimmune hemolytic anemia, Behcet's disease, Celiac disease, chronic active hepatitis, CREST syndrome, Crohn's disease, dermatomyositis, dilated cardiomyopathy, eosinophilia-myalgia syndrome, epidernolisis bullosa acquisita (EBA), giant cell arteritis, Goodpasture's syndrome, Graves' disease, Guillain-Barre syndrome, hemochromatosis, Henoch-Schonlein purpura, idiopathic IgA nephropathy, insulin-dependent diabetes mellitus (IDDM), juvenile rheumatoid arthritis, Lambert-Eaton syndrome, linear IgA dermatosis, lupus erythematosus, multiple sclerosis, myasthenia gravis, myocarditis, narcolepsy, necrotizing vasculitis, neonatal lupus syndrome (NLE), nephrotic syndrome, pemphigoid, phemphigus, polymyositis, primary sclerosing cholangitis, psoriasis, rapidly progressive glomerulonephritis (RPGN), Reiter's syndrome, rheumatoid arthritis, scleroderma, Sjogren's syndrome, stiff-man syndrome, thyroiditis, and ulcerative colitis.

The terms “infectious disease” and “infection” are used herein to refer to or describe the invasion and multiplication of microorganisms in body tissues, especially causing pathological symptoms. Examples of infectious diseases include without limitation, viral diseases and bacterial diseases, such as, e.g., HIV infection, West Nile virus infection, hepatitis A, B, and C, small pox, tuberculosis, Vesicular Stomatitis Virus (VSV) infection, Respiratory Syncytial Virus (RSV) infection, human papilloma virus (HPV) infection, SARS, influenza, Ebola, viral meningitis, herpes, anthrax, lyme disease, and E. coli infections, among others.

### EXAMPLES

Starting materials and reagents disclosed below are known to those skilled in the art, are commercially available and/or can be prepared using well-known techniques.

Materials

Chemicals were purchased from Sigma-Aldrich (USA). Oligonucleotides were from Microsynth (Switzerland). Unless stated otherwise, DNA polymerases, restriction enzymes and buffers were from New England Biolabs (USA) or Fermentas/Thermo Fisher Scientific (USA). Inducible E. coli expression strains were used for cloning and protein production, e.g. E. coli XL1-blue (Stratagene, USA) or BL21 (Novagen, USA).

Molecular Biology

Unless stated otherwise, methods are performed according to known protocols (see, e.g., Sambrook J., Fritsch E. F. and Maniatis T., Molecular Cloning: A Laboratory Manual, Cold Spring Harbor Laboratory 1989, New York).

Designed Ankyrin Repeat Protein Libraries

Methods to generate designed ankyrin repeat protein libraries have been described, e.g. in U.S. Pat. No. 7,417,130; Binz et al. 2003, loc. cit.; Binz et al. 2004, loc. cit.. By such methods designed ankyrin repeat protein libraries having randomized ankyrin repeat modules and/or randomized capping modules can be constructed. For example, such libraries could accordingly be assembled based on a fixed N-terminal capping module (e.g. the N-terminal capping module of SEQ ID NO: 5, 6 or 7) or a randomized N-terminal capping module according to SEQ ID NO: 8, one or more randomized repeat modules according to the sequence motif of SEQ ID NO: 9, 10 or 11, and a fixed C-terminal capping module (e.g. the C-terminal capping module of SEQ ID NO: 12, 13 or 14) or a randomized C-terminal capping module according to SEQ ID NO: 15. Preferably, such libraries are assembled to not have any of the amino acids C, G, M, N (in front of a G residue) and P at randomized positions of repeat or capping modules. In addition, randomized repeat modules according to the sequence motif of SEQ ID NO: 9, 10 or 11 could be further randomized at position 10 and/or position 17; the randomized N-terminal capping module according to the sequence motif of SEQ ID NO: 8 could be further randomized at position 7 and/or position 9; and the randomized C-terminal capping modules according to the sequence motif of SEQ ID NO: 15 could be further randomized at positions 10, 11 and/or 17.

Furthermore, such randomized modules in such libraries may comprise additional polypeptide loop insertions with randomized amino acid positions. Examples of such polypeptide loop insertions are complement determining region (CDR) loop libraries of antibodies or de novo generated peptide libraries. For example, such a loop insertion could be designed using the structure of the N-terminal ankyrin repeat domain of human ribonuclease L (Tanaka, N., Nakanishi, M, Kusakabe, Y, Goto, Y., Kitade, Y, Nakamura, K. T., EMBO J. 23(30), 3929-3938, 2004) as guidance. In analogy to this ankyrin repeat domain where ten amino acids are inserted in the beta-turn present close to the border of two ankyrin repeats, ankyrin repeat protein libraries may contain randomized loops (with fixed and randomized positions) of variable length (e.g. 1 to 20 amino acids) inserted in one or more beta-turns of an ankyrin repeat domain.

Any such N-terminal capping module of an ankyrin repeat protein library preferably possesses the RELLKA, RILLKA or RILLAA motif (e.g. present from position 21 to 26 in SEQ ID NO: 16) and any such C-terminal capping module of an ankyrin repeat protein library preferably possesses the KLN, KLA or KAA motif (e.g. present at the last three amino acids in SEQ ID NO: 16).

The design of such an ankyrin repeat protein library may be guided by known structures of an ankyrin repeat domain interacting with a target. Examples of such structures, identified by their Protein Data Bank (PDB) unique accession or identification codes (PDB-IDs), are 1WDY, 3V31, 3V30, 3V2X, 3V2O, 3UXG, 3TWQ-3TWX, 1N11, 1S70 and 2ZGD.

Examples of designed ankyrin repeat protein libraries, such as N2C and N3C designed ankyrin repeat protein libraries, have been described (U.S. Pat. No. 7,417,130; Binz et al. 2003, loc. cit.; Binz et al. 2004, loc. cit.). The digit in N2C and N3C describes the number of randomized repeat modules present between the N-terminal and C-terminal capping modules.

The nomenclature used to define the positions inside the repeat units and modules is based on Binz et al. 2004, loc. cit. with the modification that borders of the ankyrin repeat modules and ankyrin repeat units are shifted by one amino acid position. For example, position 1 of an ankyrin repeat module of Binz et al. 2004 (loc. cit.) corresponds to position 2 of an ankyrin repeat module of the current disclosure and consequently position 33 of an ankyrin repeat module of Binz et al. 2004, loc. cit. corresponds to position 1 of a following ankyrin repeat module of the current disclosure.

All the DNA sequences were confirmed by Sanger sequencing.

### Example 1: Selection of Binding Proteins Comprising an Ankyrin Repeat Domain with Binding Specificity for HBVc18 Peptide-MHC Complex (HBVc18pMHC)

### Summary

Using ribosome display (Hanes, J. and Plückthun, A., PNAS 94, 4937-42, 1997), multiple ankyrin repeat domains with binding specificity for HBVc18 peptide-MHC complex (HBVc18pMHC) were selected from DARPin® libraries in a way similar to the one described by Binz et al. 2004 (loc. cit.), with specific conditions and additional de-selection steps as described below. The binding and specificity of the selected clones towards recombinant HBVc18pMHC and other pMHC complexes were assessed by E. coli crude extract Homogeneous Time Resolved Fluorescence (HTRF), indicating that multiple HBVc18pMHC-specific binding proteins were successfully selected. For example, the ankyrin repeat domains of SEQ ID NO: 16 to 26 constitute amino acid sequences of selected binding proteins comprising an ankyrin repeat domain with binding specificity for HBVc18pMHC. Individual ankyrin repeat modules from such ankyrin repeat domains with binding specificity to HBVc18pMHC are provided, e.g., in SEQ ID NO: 27 to 50.

Production of Biotinylated pMHC Complexes as Target and Selection Material

Biotinylated tripartite complexes of HLA-A*0201 (SEQ ID NO: 69), human beta-2-microglobulin (hβ2m; SEQ ID NO: 70) and one of the peptides listed in Table 1 were produced according to established protocols (Garboczi et al, 1992; Celie et al, 2009).

TABLE 1
Peptides used for generation of pMHC complexes
for selection and screening of designed
ankyrin repeat proteins

| Peptide description | Peptide Sequence |
|---|---|
| HLA-A*0201: HBc18 | FLPSDFFPSV |
| HLA-A*0201: HBe183 | FLLTRILTI |
| HLA-A*0201: HBe183mut | FLLRILTI |
| HLA-A*0201: NY-ESO-1 | SLLMWITQV |
| HLA-A*0201: EBNA-1 | FMVFLQTHI |
| HLA-A*0201: human off-target 1 | ALPTLIPSV |
| HLA-A*0201: human off-target 2 | FLPDANSSV |
| HLA-A*0201: human off-target 3 | FLPQGFPDSV |
| HLA-A*0201: human off-target 4 | RLPPDFFGV |
| HLA-A*0201: human off-target 5 | YLDLFGDPSV |
| HLA-A*0201 alanine scan 1 | ALPSDFFPSV |
| HLA-A*0201 alanine scan 2 | FLASDFFPSV |
| HLA-A*0201 alanine scan 3 | FLPADFFPSV |
| HLA-A*0201 alanine scan 4 | FLPSAFFPSV |
| HLA-A*0201 alanine scan 5 | FLPSDAFPSV |
| HLA-A*0201 alanine scan 6 | FLPSDFAPSV |
| HLA-A*0201 alanine scan 7 | FLPSDFFASV |
| HLA-A*0201 alanine scan 8 | FLPSDFFPAV |

Amino acids with homology to the HBVc18 peptide are underlined.

All pMHC complexes used in experiments underwent prior quality control testing.

Selection of HBVc18pMHC-Specific Ankyrin Repeat Proteins by Ribosome Display

The selection of pMHC-specific ankyrin repeat proteins was performed by ribosome display (Hanes and Pluckthun, loc. cit.) using the HBVc18pMHC complex as a target, libraries of ankyrin repeat proteins as described above, and established protocols (See, e.g., Zahnd, C., Amstutz, P. and Pluckthun, A., Nat. Methods 4, 69-79, 2007). The number of reverse transcription (RT)-PCR cycles after each selection round was continuously reduced, adjusting to the yield due to enrichment of binders. The first four or five rounds of selection employed standard ribosome display selection, using decreasing target concentrations and increasing washing stringency to increase selection pressure from round 1 to round 4 (Binz et al. 2004, loc. cit.). Unusual de-selection steps were incorporated into the selection strategy, as described below.

During the ribosome display rounds, de-selection (or negative selection) steps were incorporated, wherein the ternary complexes were pre-incubated with the corresponding isotype HLA molecule containing another peptide, and only then transferred to the target HBVc18pMHC complex, in order to direct binding of ankyrin repeat proteins towards the peptide-embedded epitope and away from the common HLA-A scaffold. In other words, de-selection (or negative selection) was performed in order to de-select ankyrin repeat proteins that bind predominantly to the common HLA-A scaffold of pMHC complexes rather than to the specific epitope provided by the embedded peptide. Furthermore, ankyrin repeat proteins that cross-react with the epitope provided by the embedded peptide used for de-selection were also de-selected. Here, the peptides HBe183 (FLLTRILTI), HBe183mut (FLLRILTI), EBNA-1 (FMVFLQTHI) and NY-ESO-1 (SLLMWITQV) were used for de-selection.

In detail, Nunc MaxiSorp plates were coated with 100 μl solution of 66 nM neutravidin in PBS and incubated at 4° C. over night. The following day, the MaxiSorp 96-well microplate were washed three times with 300 μl PBST per well and blocked with 300 μl PBST-BSA for 1 h at 4° C., rotating at 700 rpm, prior to the de-selection step. After emptying the wells, 100 μl of a 50 nM biotinylated pMHC de-selection target solutions in PBST-BSA was added to each well, rotating with 700 rpm at 4° C., 1 h. During this incubation step, mRNA in-vitro translations were performed. Shortly after translations and generation of the ternary complex mixes the pMHC-PBST-BSA solutions were discarded and Nunc MaxiSorp microplate wells were washed three times with 300 μl PBST and finally incubated with WBT-BSA. For the actual de-selection step, the WBT-BSA solutions were discarded and aliquots of 100 μl (for selection rounds 2-5, respectively 150 μl for the first selection round) of the translated ternary complexes were transferred subsequently three times to a prepared Nunc MaxiSorp well containing the immobilized de-selection pMHC target and incubated in each of the three wells for 20 min at 4° C. At the end of the deselection process, the 100 μl of the ternary complex aliquots of each selection pool were merged again and the according volumes were taken forward into the selection of the actual target.

After the 4^{th} and 5^{th} ribosome display selection rounds, the pools were cloned with BamHI and PstI into a bacterial expression vector and transformed into E. coli XL-1 Blue and plated on LB/Glu/Amp Agar plates.

Selected clones bind specifically to HBVc18pMHC complex as determined by crude extract HTRF (data not shown).

Furthermore, the proteins were purified with conventional chromatography methods.

### Example 2: Determination of Dissociation Constants (K_{D}) of Ankyrin Repeat Proteins with Binding Specificity for HBVc18pMHC by Surface Plasmon Resonance (SPR) Analysis

In order to test the ability of the ankyrin repeat domains with binding specificity for HBVc18pMHC to function in an immune cell engager format and to bind to HBVc18pMHC in that format, selected ankyrin repeat domains were genetically linked, using conventional cloning methods, to a binding agent with binding specificity for a protein expressed on the surface of an immune cell. By this procedure, binding proteins were generated comprising an ankyrin repeat domain with binding specificity for HBVc18pMHC and further comprising a binding agent with binding specificity for CD3. These proteins were purified with conventional chromatography methods.

The binding affinities of the purified ankyrin repeat proteins on the HBVc18pMHC target were analyzed using a ProteOn Surface Plasmon Resonance (SPR) instrument (BioRad) and the measurement was performed according standard procedures known to the person skilled in the art.

Briefly, biotinylated HBVc18pMHC were diluted in PBST (PBS, pH 7.4 containing 0.005% Tween 20®) and coated on a NLC chip (BioRad) to a level of around 1000 resonance units (RU). The interaction of ankyrin repeat protein and pMHC complex was then measured by injecting 150 μl running buffer (PBS, pH 7.4 containing 0.005% Tween 20®) containing serial dilutions of ankyrin repeat proteins covering a concentration range between 1.2 nM and 100 nM, 2.5 nM and 200 nM, or 6.2 nM and 500 nM for multi-trace SPR measurements (on-rate measurement), followed by a running buffer flow for at least 60 minutes at a constant flow rate of 100 μl/min (off-rate measurement). No target regeneration was performed. The signals (i.e. resonance unit (RU) values) of the interspots were subtracted from the RU traces obtained after injection of ankyrin repeat protein. Based on the SPR traces obtained from the on-rate and off-rate measurements, the on- and off-rates of the corresponding ankyrin repeat proteins towards the pMHC complexes were determined.

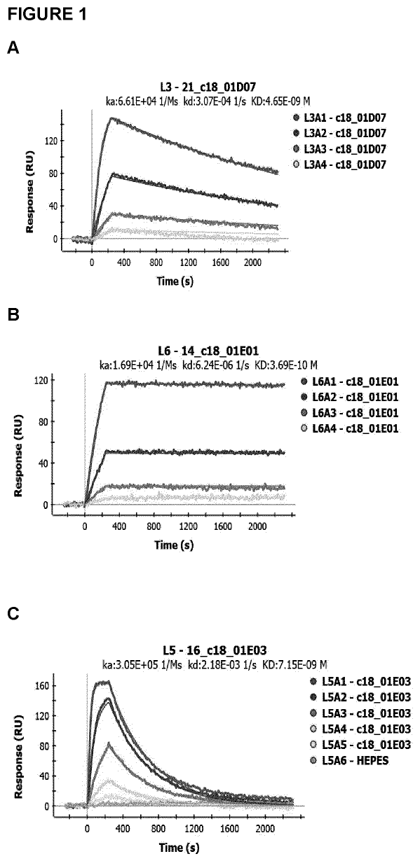

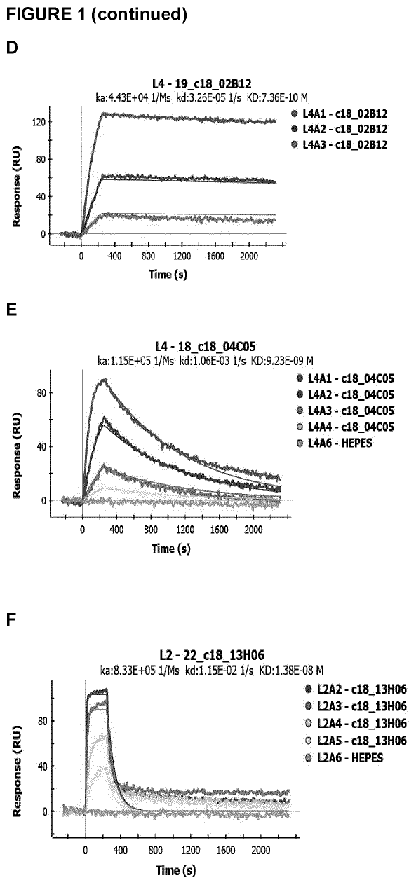

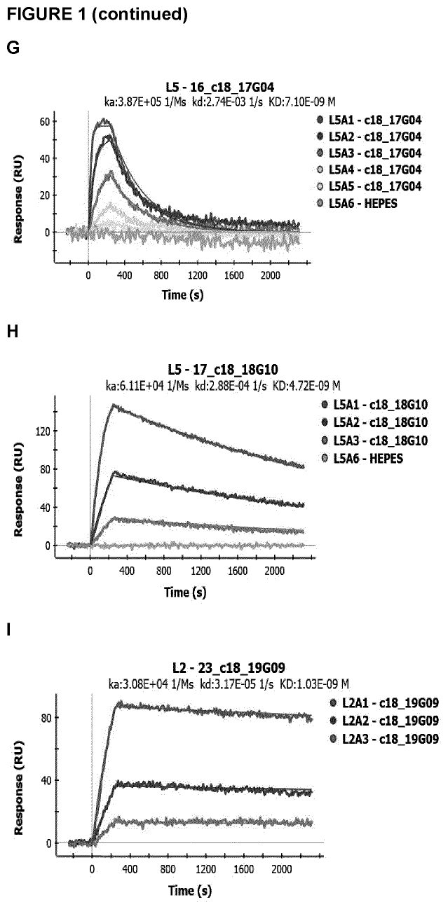

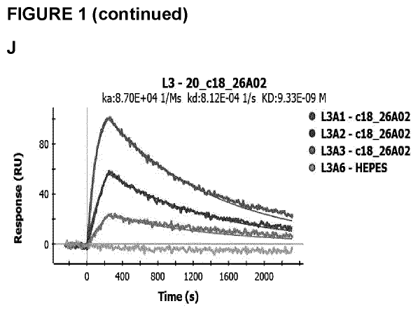

As representative examples, FIGS. 1A-J show SPR traces obtained for binding of DARPin® proteins #16 to #18 and #20 to #26, respectively, to HBVc18pMHC.

Dissociation constants (K_{D}) were calculated from the estimated on- and off-rates using standard procedures known to the person skilled in the art. K_{D} values of the binding interactions of selected ankyrin repeat proteins with HBVc18pMHC were determined to be in the low nanomolar range or below. None of these selected ankyrin repeat proteins displayed measurable binding interaction with control peptides. Table 2 provides the K_{D} values of some selected ankyrin repeat proteins as examples.

TABLE 2

| DARPin ® protein | |
|---|---|
| comprising SEQ ID NO | K_{D} [M] |
| 16 | 4.65 × 10⁻⁹ |
| 17 | 3.69 × 10⁻¹⁰ |
| 18 | 7.15 × 10⁻⁹ |
| 20 | 7.36 × 10⁻¹⁰ |
| 21 | 9.23 × 10⁻⁹ |
| 22 | 1.38 × 10⁻⁸ |
| 23 | 7.10 × 10⁻⁹ |
| 24 | 4.72 × 10⁻⁹ |
| 25 | 1.03 × 10⁻⁹ |
| 26 | 9.33 × 10⁻⁹ |

These experimental data demonstrate that designed ankyrin repeat proteins with high binding affinity and specificity for a specific pMHC complex, i.e. the HBVc18pMHC complex have been generated using screening and selection procedures as described in Example 1.

### Example 3: T Cell Activation by Binding Proteins Comprising an Ankyrin Repeat Domain with Binding Specificity for HBVc18pMHC, Using Pulsed T2 Cells

In order to test the ability of the ankyrin repeat domains with binding specificity for HBVc18pMHC to function in an immune cell engager format, selected ankyrin repeat domains were genetically linked, using conventional cloning methods, to a binding agent with binding specificity for a protein expressed on the surface of an immune cell. By this procedure, binding proteins were generated comprising an ankyrin repeat domain with binding specificity for HBVc18pMHC and further comprising a binding agent with binding specificity for CD3.

The ability of the HBVc18pMHC-specific ankyrin repeat domains in T cell engager (TCE) format to activate T cells upon binding to HBVc18pMHC-displaying target cells was analyzed in T cell activation assays. T2 cells pulsed with HBVc18 peptide were used as HBVc18pMHC-displaying target cells. The effector cells were BK112 T cells, which are monoclonal CD8⁺ T cells expanded from healthy donors (Levitsky et al., J. Immunol. 161:594-601 (1998)). For the T cell activation assay, T2 cells were pulsed overnight at 37° C. with 10 μM of peptide. Effector BK112 T cells were added at an effector to target cell ratio of 1:5 in the presence of TCE DARPin® proteins at different concentrations (e.g. 1 pM) and incubated for 4-5 hours at 37° C. Intracellular IFN-γ in the CD8+ T cells was then determined by fluorescence-activated cell sorting (FACS), as a measure of T cell activation. The reagents used for FACS included APC mouse anti-human IFN-γ and Pacific Blue™ mouse anti-human CD8 from BD Biosciences.

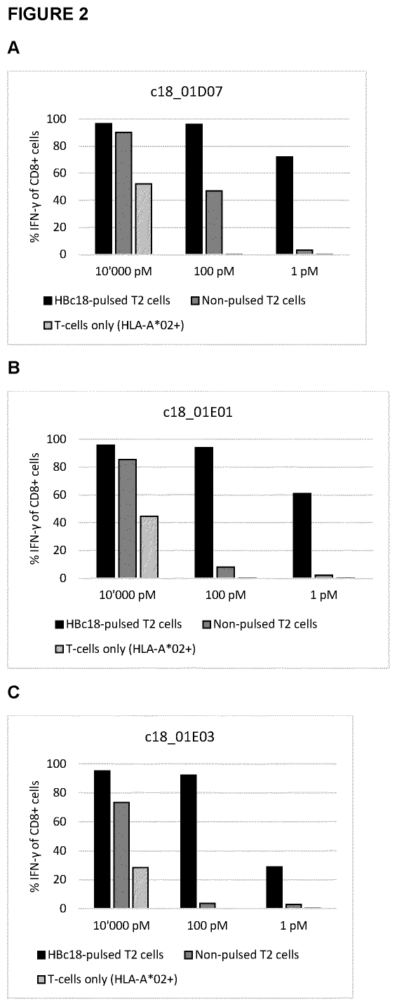

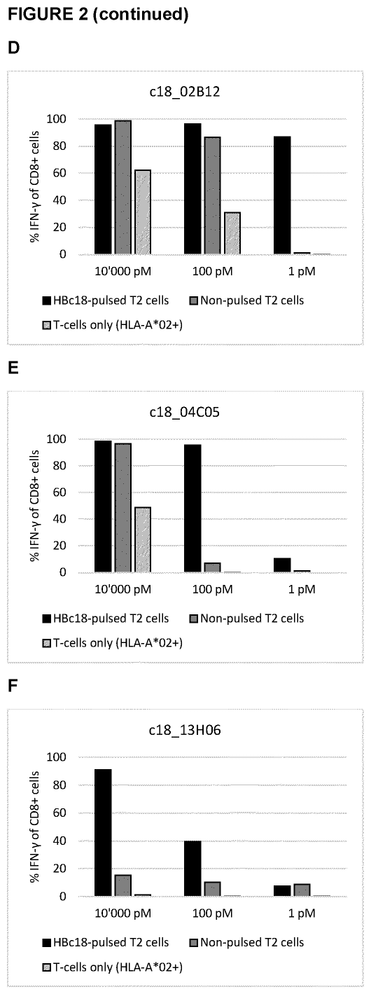

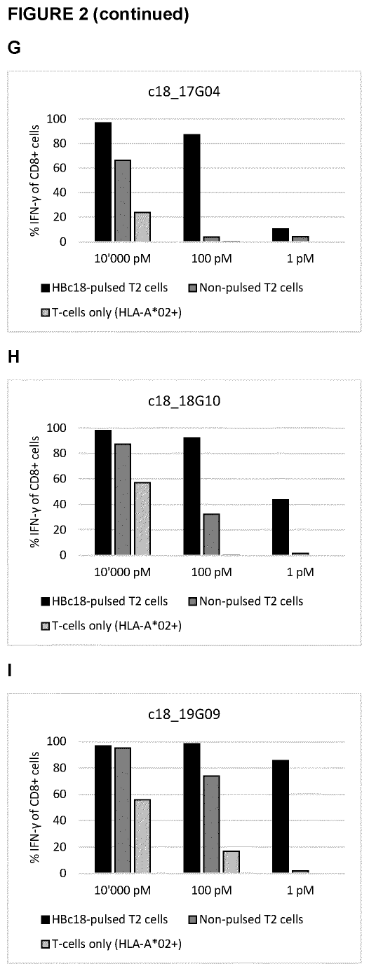

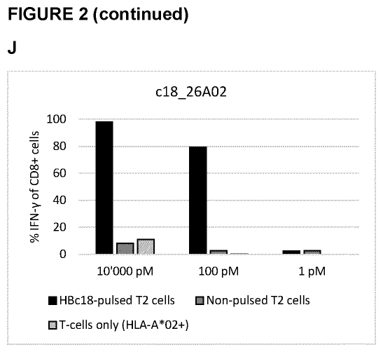

FIGS. 2A to J show the results of testing a panel of different HBVc18pMHC-specific ankyrin repeat domains in TCE format (at 1 pM, 100 pM and 10′000 pM) in this T cell activation assay. The HBVc18pMHC-specific ankyrin repeat domains in TCE format were (corresponding to the figure numbers in FIG. 2) (A) TCE DARPin® protein #16, (B) TCE DARPin® protein #17, (C) TCE DARPin® protein #18, (D) TCE DARPin® protein #20, (E) TCE DARPin® protein #21, (F) TCE DARPin® protein #22, (G) TCE DARPin® protein #23, (H) TCE DARPin® protein #24, (I) TCE DARPin® protein #25, and (J) TCE DARPin® protein #26. As a control, the experiment was also performed with non-pulsed T2 cells and T-cells only (HLA-A*02+). After 4 hours incubation, intracellular IFN-γ in the T cells was determined by FACS.

All 10 TCE DARPin® proteins specifically mediated T cell activation in the presence of target peptide-MHC complex in pulsed T2 cells, but not (or much less) in the absence of target peptide-MHC complex in non-pulsed T2 cells or in the presence of T cells only. All of these HBVc18pMHC-specific ankyrin repeat domains in TCE format achieved at least a 100-fold higher activation of effector T cells in the presence of pulsed T2 cells as compared to non-pulsed T2 cells. Thus, all of these HBVc18pMHC-specific ankyrin repeat domains in TCE format were able to specifically activate effector T cells dependent on the presence of HBVc18pMHC-displaying target cells.

### Example 4: Specificity of Target Peptide Binding by Binding Proteins Comprising an Ankyrin Repeat Domain with Binding Specificity for HBVc18pMHC, Analyzed by Alanine Scanning Mutagenesis

In order to analyze the specific interaction between the ankyrin repeat domains with binding specificity for HBVc18pMHC and the HBVc18pMHC target, alanine-scanning mutagenesis was performed. For this purpose, the HBVc18 (HBV core antigen protein 18-27) (SEQ ID NO: 51) peptide and a series of variants thereof, in each of which one amino acid of the HBVc18 peptide was replaced with alanine, were tested in a T cell activation assay. The assay used pulsed T2 cells as target cells and BK112 T cells as effector cells, essentially as described in Example 3. If an amino acid residue in the HBVc18 peptide is important for the binding interaction between the ankyrin repeat domain with binding specificity for HBVc18pMHC and the HBVc18pMHC target, then a reduced or no T cell activation will be observed if this amino acid residue is replaced with alanine.

The alanine-mutated peptides were obtained from Genscript and had the following sequences: ALPSDFFPSV (SEQ ID NO: 61; F to A at position 1), FLASDFFPSV (SEQ ID NO: 62; P to A at position 3), FLPADFFPSV (SEQ ID NO: 63; S to A at position 4), FLPSAFFPSV (SEQ ID NO: 64; D to A at position 5), FLPSDAFPSV (SEQ ID NO: 65; F to A at position 6), FLPSDFAPSV (SEQ ID NO: 66; F to A at position 7), FLPSDFFASV (SEQ ID NO: 67; P to A at position 8), and FLPSDFFPAV (SEQ ID NO: 68; S to A at position 9). T2 cells were pulsed with the HBVc18 peptide and each of the mutated peptides (10 μM). Pulsed T2 cells were incubated with effector BK112 T cells at an effector to target cell ratio of 1:5 in the presence of TCE DARPin® protein.

The alanine scanning mutagenesis analysis showed which peptide residues were important for the binding interaction between the TCE DARPin® proteins and the HBVc18pMHC target (FIGS. 3A-J). These data demonstrate that in each case at least 2 peptide residues were important for this binding interaction, causing a decrease in T cell activation by at least 50% as compared to the HBVc18 peptide when mutated to alanine. Surprisingly, in some cases, interactions with at least 4 peptide residues were important for binding of the ankyrin repeat domain to the target peptide-MHC complex, i.e. the mutation of which to alanine resulted in a reduction of T cell activation by at least 50% compared to the wild-type peptide. As an example, for DARPin® protein #23, at least positions 5, 6, 7, and 8 of the HBVc18 target peptide were important for the functional binding interaction leading to T cell activation (FIG. 3G). As another example, for DARPin® protein #21, at least positions 5, 6, 7, and 8 of the HBVc18 target peptide were important for the functional binding interaction leading to T cell activation (FIG. 3E). As another example, for DARPin® protein #22, at least positions 3, 6, 7 and 8 of the HBVc18 target peptide were important for the functional binding interaction leading to T cell activation (FIG. 3F).

In conclusion, these data show that a surprisingly large number of peptide residues are important for the specific interaction between binding proteins comprising an ankyrin repeat domain with binding specificity for HBVc18pMHC and the HBVc18 target peptide. This may reflect a structural difference between the binding surface formed by the designed ankyrin repeat domains and that formed by other binding proteins, such as antibodies and T cell receptors (TCRs).

### Example 5: Influence of Linker Length on Immune Cell Activation by Binding Proteins Comprising an Ankyrin Repeat Domain with Binding Specificity for NYESOpMHC

In order to test the influence of the length of the linker between the pMHC-specific binding domain and the CD3-specific binding agent in a binding protein of the invention in T cell engager (TCE) format on the potency of such binding proteins in TCE format, different constructs comprising a NYESOpMHC binding domain connected to a CD3 binding domain with linkers of varying length were tested in a T cell activation assay using different tumor cell lines.

Linker Length Design & Abbreviations

The following binding proteins in TCE format were generated comprising DARPin® protein #78 (SEQ ID NO: 78) or DARPin® protein #79 (SEQ ID NO: 79) as the NYESOpMHC-specific binding domain:

TABLE 3
TCE binding proteins and the linkers used

| Binding protein in TCE format | PT linker |
|---|---|
| TCE DARPin ® protein #78 (standard) | standard |
| TCE DARPin ® protein #78 (XXS) | XXS |
| TCE DARPin ® protein #78 (XS) | XS |
| TCE DARPin ® protein #78 (S) | S |
| TCE DARPin ® protein #78 (L) | L |
| TCE DARPin ® protein #79 (standard) | standard |
| TCE DARPin ® protein #79 (XXS) | XXS |
| TCE DARPin ® protein #79 (XS) | XS |
| TCE DARPin ® protein #79 (S) | S |
| TCE DARPin ® protein #79 (L) | L |

TABLE 4
Overview of the linkers tested in the
binding proteins of Table 3

| PT linker | SEQ | Length | |
|---|---|---|---|
| abbreviation | ID NO | (a.a.) | Amino Acid Sequence |
| standard | 1 | 24 | GSPTPTPTTPTPTPTTPTPTPTGS |
| XXS | 77 | 6 | GSPTGS |
| XS | 76 | 11 | GSPTPTPTTGS |
| S | 75 | 18 | GSPTPTPTTPTPTPTTGS |
| L | 74 | 38 | GSPTPTPTTPTPTPTTPTPTPTTPTP |
| | | | TPTTPTPTPTGS |

Potency and Specificity of Binding Proteins in TCE Format in T Cell Activation Assays

The potency and specificity of binding proteins in TCE format generated with different linkers was tested using different tumor cell lines in a T cell activation assay. Some of the tumor cell lines (IM9 and U266B1) endogenously express NY-ESO-1 and present the NY-ESO-1 target peptide in a pMHC complex on their cell surface. Other tumor cell lines (MCF7 and Colo205) do not express NY-ESO-1 and hence do no present the NY-ESO-1 target peptide in a pMHC complex on their cell surface.

The T cell activation assay was performed by methodology known in the art. Briefly, target tumor cells and effector PBMCs were combined at an effector-to-target cell ratio of 5:1, NYESOpMHC-specific ankyrin repeat domains in TCE format (see constructs in Table 3) were added at different concentrations, and the mixtures were incubated for 48 hours at 37° C. The levels of activation marker CD25 on CD8+ T cells were determined by FACS (using CD25 Monoclonal Antibody (BC96), PerCP-Cyanine5.5, eBioscience™; and Alexa Fluor® 488 mouse anti-human CD8 from BD Biosciences). Immune cell activation was compared using a tumor cell line (IM9) that endogenously expresses HLA-A2 and NY-ESO-1 and displays NYESOpMHC on its surface, and a tumor cell line (MCF-7) that expresses HLA-A2 but not NY-ESO-1 and does not display NYESOpMHC on its surface. PBMCs isolated from a donor were used as effector cells. Similar T cell activation assays were also performed using another tumor cell line (U266B1) that endogenously expresses HLA-A2 and NY-ESO-1 and displays NYESOpMHC on its surface, and another tumor cell line (Colo205) that expresses HLA-A2 but not NY-ESO-1 and does not display NYESOpMHC on its surface. As a control, effector PBMCs were treated identically, except that no target cells were added.

The levels of T cell activation obtained in the presence of the binding proteins are shown in FIGS. 4 to 7. Enhanced potency was observed with decreasing linker length. The highest potency was observed for the constructs designed with the shortest linker (XXS), with the potency of the constructs designed with the second shortest linker (XS) also strongly enhanced compared to the constructs designed with longer linkers. No increase in the levels of non-specific activation was observed for any of the cell lines tested.

The EC50 values obtained from the data shown in FIG. 4 are listed in Table 5 below.

TABLE 5
EC50 values obtained from the data shown in FIG. 4

| | EC50 [pM] |
|---|---|
| Binding protein in TCE format | IM9 (Ag+) |
| TCE DARPin ® protein #78 (L) | 1245 |
| TCE DARPin ® protein #78 (standard) | 365.5 |
| TCE DARPin ® protein #78 (S) | 422.1 |
| TCE DARPin ® protein #78 (XS) | 304.6 |
| TCE DARPin ® protein #78 (XXS) | 143.4 |

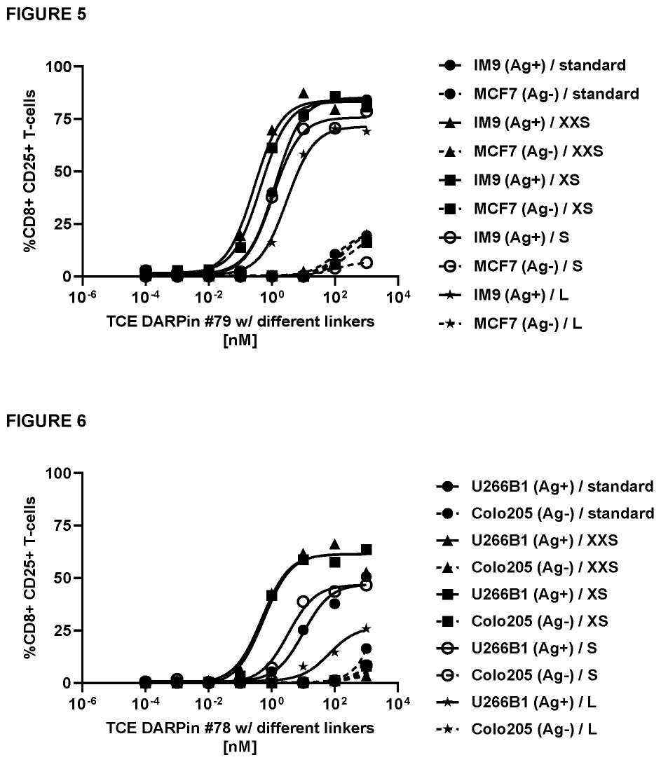

The EC50 values obtained from the data shown in FIG. 5 are listed in Table 6 below.

TABLE 6
EC50 values obtained from the data shown in FIG. 5

| | EC50 [pM] |
|---|---|
| Binding protein in TCE format | IM9 (Ag+) |
| TCE DARPin ® protein #79 (L) | 2949 |
| TCE DARPin ® protein #79 (standard) | 1236 |
| TCE DARPin ® protein #79 (S) | 1075 |
| TCE DARPin ® protein #79 (XS) | 437.3 |
| TCE DARPin ® protein #79 (XXS) | 272.8 |

The EC50 values obtained from the data shown in FIG. 6 are listed in Table 7 below.

TABLE 7
EC50 values obtained from the data shown in FIG. 6

| | EC50 [pM] |
|---|---|
| Binding protein in TCE format | U266B1 (Ag+) |
| TCE DARPin ® protein #78 (L) | ~67000 |
| TCE DARPin ® protein #78 (standard) | 10120 |
| TCE DARPin ® protein #78 (S) | 3361 |
| TCE DARPin ® protein #78 (XS) | 558.9 |
| TCE DARPin ® protein #78 (XXS) | 491.8 |

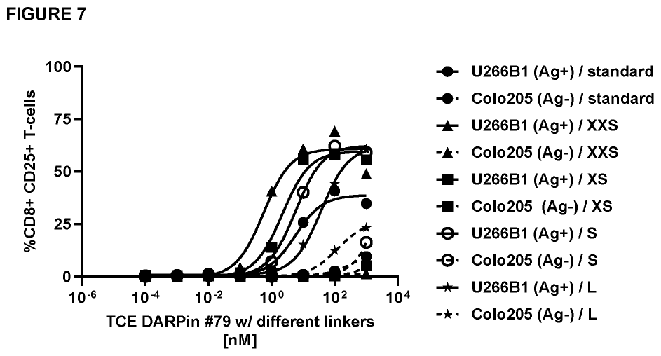

The EC50 values obtained from the data shown in FIG. 7 are listed in Table 8 below.

TABLE 8
EC50 values obtained from the data shown in FIG. 7

| | EC50 [pM] |
|---|---|
| Binding protein in TCE format | U266B1 (Ag+) |
| TCE DARPin ® protein #79 (L) | 37480 |
| TCE DARPin ® protein #79 (standard) | 5655 |
| TCE DARPin ® protein #79 (S) | 5776 |
| TCE DARPin ® protein #79 (XS) | 2188 |
| TCE DARPin ® protein #79 (XXS) | 555.2 |

From the data, it can be concluded that, for the pMHC-specific binding domains in TCE format, preferably a linker is used that is at least as short as the L linker, more preferably at least as short as the standard linker, more preferably at least as short as the S linker, more preferably at least as short as the XS linker, more preferably at least as short as short as the XXS linker and most preferably as short or about as short as the XXS linker. The specific linkers provided in this Example are examples of such linkers with the preferred lengths.

In similar experiments performed with HBVc18pMHC and HBVc18pMHC-specific ankyrin repeat domains in TCE format (TCE binding proteins) of the invention, similar effects of linker length are observed.

1. A method of producing a peptide-MHC (pMHC)-specific binding protein, wherein said binding protein comprises a designed repeat domain with binding specificity for a target peptide-MHC complex, the method comprising the steps of:
(a) providing a collection of designed repeat domains;
(b) providing a recombinant target peptide-MHC complex; and
(c) screening said collection of designed repeat domains for specific binding to said target peptide-MHC complex to obtain at least one designed repeat domain with binding specificity for said target peptide-MHC complex.

2. The method of claim 1, wherein said designed repeat domain is a designed ankyrin repeat domain.

3. The method of claim 1 or 2, wherein said collection comprises designed repeat domains that comprise fixed positions and randomized positions, and wherein designed repeat domains of said collection differ from each other in at least one of the randomized positions.

4. (canceled)

5. The method of claim 1, wherein the method further comprises the steps of (i) providing a second recombinant peptide-MHC complex, wherein said peptide of said second peptide-MHC complex comprises an amino acid sequence that differs from said target peptide by at least one amino acid residue; and (ii) removing from said collection by negative selection designed repeat domains with binding specificity for said second recombinant peptide-MHC complex.

6. The method of claim 1, wherein said repeat domain with binding specificity for a target peptide-MHC complex binds to said target peptide-MHC complex in PBS with a dissociation constant (K_{D}) below 10⁻⁷M.

7. The method of claim 1, wherein binding of said repeat domain with binding specificity for a target peptide-MHC complex to said target peptide-MHC complex comprises interaction of said repeat domain with at least two amino acid residues of said target peptide.

8.-10. (canceled)

11. A recombinant binding protein comprising a designed repeat domain obtainable by the method of claim 1.

12. A recombinant binding protein comprising a first designed repeat domain, wherein said first repeat domain has binding specificity for a first target peptide-MHC complex, wherein preferably said first target peptide is selected from the group consisting of (i) a peptide derived from a protein expressed in a tumor cell, (ii) a peptide derived from a protein of an infectious agent, preferably a viral infectious agent, and (iii) a peptide derived from a protein associated with an autoimmune disorder.

13. (canceled)

14. The binding protein of claim 12, wherein said first repeat domain binds to said first target peptide-MHC complex in PBS with a dissociation constant (K_{D}) below 10⁻⁷M.

15. The binding protein of claim 12, wherein binding of said first repeat domain to said first target peptide-MHC complex comprises interaction of said first repeat domain with at least one, at least two amino acid residues of said first target peptide.

16. (canceled)

17. The binding protein of claim 12, wherein said first MHC is MHC class I.

18. (canceled)

19. The binding protein of claim 12, wherein said first target peptide is derived from HBV core antigen (HBcAg).

20. (canceled)

21. The binding protein of claim 12, wherein said first repeat domain is a designed ankyrin repeat domain.

22. The binding protein of claim 21, wherein said first ankyrin repeat domain comprises an ankyrin repeat module comprising an amino acid sequence selected from the group consisting of (1) SEQ ID NOs: 27 to 50 and (2) sequences in which up to 9 amino acids in any of SEQ ID NOs: 27 to 50 are substituted by another amino acid.

23. The binding protein of claim 21, wherein said first ankyrin repeat domain comprises an amino acid sequence with at least 80% amino acid sequence identity with any one of SEQ ID NOs: 16 to 26, wherein G at position 1 and/or S at position 2 of SEQ ID NOs: 16 to 26 are optionally missing, and wherein A at the second last position of SEQ ID NOs: 16 to 26 is optionally substituted by L and/or A at the last position of SEQ ID NOs: 16 to 26 is optionally substituted by N.

24. The binding protein of claim 12, wherein said binding protein further comprises a second designed repeat domain, wherein said second repeat domain has binding specificity for a second target peptide-MHC complex, wherein preferably said second target peptide is selected from the group consisting of (i) a peptide derived from a protein expressed in a tumor cell, (ii) a peptide derived from a protein of an infectious agent, preferably a viral infectious agent, and (iii) a peptide derived from a protein associated with an autoimmune disorder.

25.-40. (canceled)

41. The binding protein of claim 12, wherein said binding protein further comprises a binding agent with binding specificity for a protein expressed on the surface of an immune cell.

42.-51. (canceled)

52. A nucleic acid encoding the designed repeat domain with binding specificity for a first target peptide-MHC complex as defined in claim 12.

53. A pharmaceutical composition comprising the binding protein of claim 12 and a pharmaceutically acceptable carrier and/or diluent.

54.-55. (canceled)

56. A method of treating a medical condition, the method comprising the step of administering to a patient in need thereof a therapeutically effective amount of the binding protein of claim 12.

57.-67. (canceled)


SEQUENCE LISTING (79 sequences)

SEQ ID NO: 1 - protein, 24 aa, Artificial Sequence
  PT-rich peptide linker
GSPTPTPTTPTPTPTTPTPTPTGS

SEQ ID NO: 2 - protein, 22 aa, Artificial Sequence
  PT-rich peptide linker
GSPTPTPTTPTPTPTTPTPTPT

SEQ ID NO: 3 - protein, 5 aa, Artificial Sequence
  Consensus GS linker
  VARIANT 1..5: [Gly-Gly-Gly-Gly-Ser]n, wherein n is 1, 2, 3, 4, 5, or 6
GGGGS

SEQ ID NO: 4 - protein, 10 aa, Artificial Sequence
  His-tag
MRGSHHHHHH

SEQ ID NO: 5 - protein, 32 aa, Artificial Sequence
  N-cap
GSDLGQKLLTAAIVGQDDEVRILLAAGADVNA

SEQ ID NO: 6 - protein, 32 aa, Artificial Sequence
  N-cap
GSDLGQKLLTAAIVGQDDEVRILLKAGADVNA

SEQ ID NO: 7 - protein, 32 aa, Artificial Sequence
  N-cap
GSDLGQKLLTAAIVGQDDEVRELLKAGADVNA

SEQ ID NO: 8 - protein, 32 aa, Artificial Sequence
  N-Cap (randomized)
  VARIANT 6: Xaa can be any naturally occuring amino acid
  VARIANT 10: Xaa can be any naturally occuring amino acid
  VARIANT 13: Xaa can be any naturally occuring amino acid
  VARIANT 14: Xaa can be any naturally occuring amino acid
GSDLGXKLLXAAXXGQDDEVRILLAAGADVNA

SEQ ID NO: 9 - protein, 33 aa, Artificial Sequence
  Randomized repeat module
  VARIANT 1: Xaa can be any naturally occuring amino acid
  VARIANT 3: Xaa can be any naturally occuring amino acid
  VARIANT 4: Xaa can be any naturally occuring amino acid
  VARIANT 6: Xaa can be any naturally occuring amino acid
  VARIANT 14: Xaa can be any naturally occuring amino acid
  VARIANT 15: Xaa can be any naturally occuring amino acid
  VARIANT 27: Xaa can be any naturally occuring amino acid
XDXXGXTPLHLAAXXGHLEIVEVLLKXGADVNA

SEQ ID NO: 10 - protein, 33 aa, Artificial Sequence
  Randomized repeat module
  VARIANT 3: Xaa can be any naturally occuring amino acid
  VARIANT 4: Xaa can be any naturally occuring amino acid
  VARIANT 6: Xaa can be any naturally occuring amino acid
  VARIANT 14: Xaa can be any naturally occuring amino acid
  VARIANT 15: Xaa can be any naturally occuring amino acid
KDXXGXTPLHLAAXXGHLEIVEVLLKAGADVNA

SEQ ID NO: 11 - protein, 33 aa, Artificial Sequence
  Randomized repeat module
  VARIANT 3: Xaa can be any naturally occuring amino acid
  VARIANT 4: Xaa can be any naturally occuring amino acid
  VARIANT 6: Xaa can be any naturally occuring amino acid
  VARIANT 11: Xaa can be any naturally occuring amino acid
  VARIANT 14: Xaa can be any naturally occuring amino acid
  VARIANT 15: Xaa can be any naturally occuring amino acid
KDXXGXTPLHXAAXXGHLEIVEVLLKAGADVNA

SEQ ID NO: 12 - protein, 28 aa, Artificial Sequence
  C-cap
QDKFGKTPADIAADNGHEDIAEVLQKLN

SEQ ID NO: 13 - protein, 28 aa, Artificial Sequence
  C-cap
QDQFGITPADLAAAVGHEDIAEVLQKAA

SEQ ID NO: 14 - protein, 28 aa, Artificial Sequence
  C-cap
QDYTGHTPADLAALIGHEDIAEVLQKAA

SEQ ID NO: 15 - protein, 28 aa, Artificial Sequence
  C-cap (randomized)
  VARIANT 3: Xaa can be any naturally occurding amino acid
  VARIANT 4: Xaa can be any naturally occurding amino acid
  VARIANT 6: Xaa can be any naturally occurding amino acid
  VARIANT 14: Xaa can be any naturally occurding amino acid
  VARIANT 15: Xaa can be any naturally occurding amino acid
QDXXGXTPADLAAXXGHEDIAEVLQKLN

SEQ ID NO: 16 - protein, 126 aa, Artificial Sequence
  Ankyrin repeat domain specific for HBVc18 pMHC
GSDLGHKLLFAASAGQDDEVRILLAAGADVNAKDESGQTPLHYAAFVGHLEIVEVLLKAGADVNAKDQWGHTPLHVAATFGHLEIVEVLLKAGADVNAQDTWGTTPADLAATFGHEDIAEVLQKAA

SEQ ID NO: 17 - protein, 125 aa, Artificial Sequence
  Ankyrin repeat domain specific for HBVc18 pMHC
GSDLGQKLLLAAAIGQDDEVRILLAAGADVNAKDESGHTPLHYAAHVGHLEIVEVLLKAGDVNAKDHRGFTPLHVAAQIGHLEIVEVLLKAGADVNAQDNWGITPADLAAWWGHEDIAEVLQKAA

SEQ ID NO: 18 - protein, 126 aa, Artificial Sequence
  Ankyrin repeat domain specific for HBVc18 pMHC
GSDLGYKLLTAAIVGQDDEVRILLAAGADVNAKDQSGLTPLHLAAAYGHLEIVEVLLKAGADVNAKDKHGRTPLHLAALEGHLEIVEVLLKAGADVNAQDQFGITPADLAAAVGHEDIAEVLQKAA

SEQ ID NO: 19 - protein, 126 aa, Artificial Sequence
  Ankyrin repeat domain specific for HBVc18 pMHC
GSDLGYKLLYAAWLGQDDEVRILLAAGADVNAKDHWGHTPLHLAAWIGHLEIVEVLLKAGADVNAKDSWGSTPLHLAAAWGHLEIVEVLLKAGADVNAQDTTGNTPADLAAHIGHEDIAEVLQKAA

SEQ ID NO: 20 - protein, 126 aa, Artificial Sequence
  Ankyrin repeat domain specific for HBVc18 pMHC
GSDLGFKLLEAALEGQDDEVRILLAAGADVNAKDESGQTPLHYAAFVGHLEIVEVLLKAGADVNAKDQWGHTPLHVAATFGHLEIVEVLLKAGADVNAQDTWGTTPADLAATFGHEDIAEVLQKAA

SEQ ID NO: 21 - protein, 126 aa, Artificial Sequence
  Ankyrin repeat domain specific for HBVc18 pMHC
GSDLGIKLLFAANFGQDDEVRILLAAGADVNAKDQSGRTPLHLAALWGHLEIVEVLLKAGADVNAKDVFGLTPLHIAATFGHLEIVEVLLKAGVDVNAQDYTGHTPADLAALIGHEDIAEVLQKAA

SEQ ID NO: 22 - protein, 159 aa, Artificial Sequence
  Ankyrin repeat domain specific for HBVc18 pMHC
GSDLGKKLLSAAQSGQDDEVRILLGAGADVNAKDKWGHTPLHLAAVKGHLEIAEVLLKAGADVNAKDEWGSTPLHLAASQGHLEIVEVLLKAGADVNAKDKKGATPLHLAALVGHLEIVEVLLKAVADVNAQDKSGKTPADLAARAGHQDIAEVLQKAA

SEQ ID NO: 23 - protein, 126 aa, Artificial Sequence
  Ankyrin repeat domain specific for HBVc18 pMHC
GSDLGQKLLTAAIVGQDDEVRILLAAGADVNAKDQSGLTPLHLAAAYGHLEIVEVLLKAGADVNAKDKHGRTPLHLAALEGHLEIVEVLLKAGADVNAQDQFGITPADLAAAVGHEDIAEVLQKAA

SEQ ID NO: 24 - protein, 126 aa, Artificial Sequence
  Ankyrin repeat domain specific for HBVc18 pMHC
GSDLGWKLLHAATTGQDDEVRILLAAGADVNAKDESGQTPLHYAAFVGHLEIVEVLLKAGADVNAKDQWGHTPLHVAATFGHLEIVEVLLKAGADVNAQDTWGTTPADLAATFGHEDIAEVLQKAA

SEQ ID NO: 25 - protein, 126 aa, Artificial Sequence
  Ankyrin repeat domain specific for HBVc18 pMHC
GSDLGSKLLYAAISGQDDEVRILLAAGADVNAKDESGQTPLHYAAFVGHLEIVEVLLKAGADVNAKDQWGHTPLHVAATFGHLEIVEVLLKAGADVNAQDTWGTTPADLAATFGHEDIAVVLQKAA

SEQ ID NO: 26 - protein, 159 aa, Artificial Sequence
  Ankyrin repeat domain specific for HBVc18 pMHC
GSDLGKKLLQAARAGQLDEVHELLKAGADVNAKDQHGKTPMHLAAQIGHLEIVEVLLKAGADVNAKDHIGWTPLHLAASVGHLEIVEVLLKAGADVNAKDQEGWTPLHVAAQLGHLEIVEVLLKAGADVNAQDKSGKTPADLAARAGHQDSAEVLQKAA

SEQ ID NO: 27 - protein, 33 aa, Artificial Sequence
  Ankyrin repeat module
KDESGQTPLHYAAFVGHLEIVEVLLKAGADVNA

SEQ ID NO: 28 - protein, 33 aa, Artificial Sequence
  Ankyrin repeat module
KDQWGHTPLHVAATFGHLEIVEVLLKAGADVNA

SEQ ID NO: 29 - protein, 32 aa, Artificial Sequence
  Ankyrin repeat module
KDESGHTPLHYAAHVGHLEIVEVLLKAGDVNA

SEQ ID NO: 30 - protein, 33 aa, Artificial Sequence
  Ankyrin repeat module
KDHRGFTPLHVAAQIGHLEIVEVLLKAGADVNA

SEQ ID NO: 31 - protein, 33 aa, Artificial Sequence
  Ankyrin repeat module
KDQSGLTPLHLAAAYGHLEIVEVLLKAGADVNA

SEQ ID NO: 32 - protein, 33 aa, Artificial Sequence
  Ankyrin repeat module
KDKHGRTPLHLAALEGHLEIVEVLLKAGADVNA

SEQ ID NO: 33 - protein, 33 aa, Artificial Sequence
  Ankyrin repeat module
KDHWGHTPLHLAAWIGHLEIVEVLLKAGADVNA

SEQ ID NO: 34 - protein, 33 aa, Artificial Sequence
  Ankyrin repeat module
KDSWGSTPLHLAAAWGHLEIVEVLLKAGADVNA

SEQ ID NO: 35 - protein, 33 aa, Artificial Sequence; identical to SEQ ID NO: 27
  Ankyrin repeat module

SEQ ID NO: 36 - protein, 33 aa, Artificial Sequence; identical to SEQ ID NO: 28
  Ankyrin repeat module

SEQ ID NO: 37 - protein, 33 aa, Artificial Sequence
  Ankyrin repeat module
KDQSGRTPLHLAALWGHLEIVEVLLKAGADVNA

SEQ ID NO: 38 - protein, 33 aa, Artificial Sequence
  Ankyrin repeat module
KDVFGLTPLHIAATFGHLEIVEVLLKAGVDVNA

SEQ ID NO: 39 - protein, 33 aa, Artificial Sequence
  Ankyrin repeat module
KDKWGHTPLHLAAVKGHLEIAEVLLKAGADVNA

SEQ ID NO: 40 - protein, 33 aa, Artificial Sequence
  Ankyrin repeat module
KDEWGSTPLHLAASQGHLEIVEVLLKAGADVNA

SEQ ID NO: 41 - protein, 33 aa, Artificial Sequence
  Ankyrin repeat module
KDKKGATPLHLAALVGHLEIVEVLLKAVADVNA

SEQ ID NO: 42 - protein, 33 aa, Artificial Sequence; identical to SEQ ID NO: 31
  Ankyrin repeat module

SEQ ID NO: 43 - protein, 33 aa, Artificial Sequence; identical to SEQ ID NO: 32
  Ankyrin repeat module

SEQ ID NO: 44 - protein, 33 aa, Artificial Sequence; identical to SEQ ID NO: 27
  Ankyrin repeat module

SEQ ID NO: 45 - protein, 33 aa, Artificial Sequence; identical to SEQ ID NO: 28
  Ankyrin repeat module

SEQ ID NO: 46 - protein, 33 aa, Artificial Sequence; identical to SEQ ID NO: 27
  Ankyrin repeat module

SEQ ID NO: 47 - protein, 33 aa, Artificial Sequence; identical to SEQ ID NO: 28
  Ankyrin repeat module

SEQ ID NO: 48 - protein, 33 aa, Artificial Sequence
  Ankyrin repeat module
KDQHGKTPMHLAAQIGHLEIVEVLLKAGADVNA

SEQ ID NO: 49 - protein, 33 aa, Artificial Sequence
  Ankyrin repeat module
KDHIGWTPLHLAASVGHLEIVEVLLKAGADVNA

SEQ ID NO: 50 - protein, 33 aa, Artificial Sequence
  Ankyrin repeat module
KDQEGWTPLHVAAQLGHLEIVEVLLKAGADVNA

SEQ ID NO: 51 - protein, 10 aa, Hepatitis B virus
  HBV core antigen protein 18-27
FLPSDFFPSV

SEQ ID NO: 52 - protein, 9 aa, Hepatitis B virus
  HBV envelope protein 183-191
FLLTRILTI

SEQ ID NO: 53 - protein, 8 aa, Artificial Sequence
  HBe183 peptide mutated
FLLRILTI

SEQ ID NO: 54 - protein, 9 aa, Artificial Sequence
  NY-ESO-1 peptide
SLLMWITQV

SEQ ID NO: 55 - protein, 9 aa, Eppstein Barr Virus
  Epstein-Barr nuclear antigen (EBNA)-1
FMVFLQTHI

SEQ ID NO: 56 - protein, 9 aa, Homo sapiens
  Peptide (human off-target)
ALPTLIPSV

SEQ ID NO: 57 - protein, 9 aa, Homo sapiens
  Peptide (human off-target)
FLPDANSSV

SEQ ID NO: 58 - protein, 10 aa, Homo sapiens
  Peptide (human off-target)
FLPQGFPDSV

SEQ ID NO: 59 - protein, 9 aa, Homo sapiens
  Peptide (human off-target)
RLPPDFFGV

SEQ ID NO: 60 - protein, 10 aa, Homo sapiens
  Peptide (human off-target)
YLDLFGDPSV

SEQ ID NO: 61 - protein, 10 aa, Artificial Sequence
  HBc18 peptide (alanine mutated)
ALPSDFFPSV

SEQ ID NO: 62 - protein, 10 aa, Artificial Sequence
  HBc18 peptide (alanine mutated)
FLASDFFPSV

SEQ ID NO: 63 - protein, 10 aa, Artificial Sequence
  HBc18 peptide (alanine mutated)
FLPADFFPSV

SEQ ID NO: 64 - protein, 10 aa, Artificial Sequence
  HBc18 peptide (alanine mutated)
FLPSAFFPSV

SEQ ID NO: 65 - protein, 10 aa, Artificial Sequence
  HBc18 peptide (alanine mutated)
FLPSDAFPSV

SEQ ID NO: 66 - protein, 10 aa, Artificial Sequence
  HBc18 peptide (alanine mutated)
FLPSDFAPSV

SEQ ID NO: 67 - protein, 10 aa, Artificial Sequence
  HBc18 peptide (alanine mutated)
FLPSDFFASV

SEQ ID NO: 68 - protein, 10 aa, Artificial Sequence
  HBc18 peptide (alanine mutated)
FLPSDFFPAV

SEQ ID NO: 69 - protein, 277 aa, Homo sapiens [sequence omitted]

SEQ ID NO: 70 - protein, 100 aa, Homo sapiens [sequence omitted]

SEQ ID NO: 71 - protein, 11 aa, Artificial Sequence
  Peptide linker
GSGGSGGSAGG

SEQ ID NO: 72 - protein, 15 aa, Artificial Sequence
  Avi-tag
GLNDIFEAQKIEWHE

SEQ ID NO: 73 - protein, 303 aa, Artificial Sequence
  HLA-A*0201avi
MGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPGSGGSGGSAGGGLNDIFEAQKIEWHE

SEQ ID NO: 74 - protein, 38 aa, Artificial Sequence
  Linker L
GSPTPTPTTPTPTPTTPTPTPTTPTPTPTTPTPTPTGS

SEQ ID NO: 75 - protein, 18 aa, Artificial Sequence
  Linker S
GSPTPTPTTPTPTPTTGS

SEQ ID NO: 76 - protein, 11 aa, Artificial Sequence
  Linker XS
GSPTPTPTTGS

SEQ ID NO: 77 - protein, 6 aa, Artificial Sequence
  Linker XXS
GSPTGS

SEQ ID NO: 78 - protein, 159 aa, Artificial Sequence
  Ankyrin repeat domain specific for NY-ESO-1 pMHC
GSDLGKKLLQAARAGQLDEVRELLKAGADVNAKDLIGVTPLHLAAFSGHLEIVEVLLKASADVNAKDVSGRTPLHVAAKHGHLEIVEVLLKAGADVNAKDLIGFTPLHLAAQFGHLEIVEVLLKAGADVNAQDKSGKTPADLAARAGHQDIAEVLQKAA

SEQ ID NO: 79 - protein, 158 aa, Artificial Sequence
  Ankyrin repeat domain specific for NY-ESO-1 pMHC
GSDLGKKLLQAARAGQLDEVRELLKAGADVNAKDYFGSTPLHLAQLGHLEIVEVLLKAGADVNAKDYQGHTPLHVDAFHGHLEIVEVLLKAGADVNAKDQWGVTPLHLAAEWGHLEIVEVLLKAGADVNAQDKSGKTPADLAARAGHQDIAEVLQKAA